(19) United States
(12) Patent Application Publication
(10) Pub. No.: US 2010/0061976 A1
(43) Pub. Date: Mar. 11, 2010

(54) Method for treating or preventing osteoporosis by reducing follicle stimulating hormone to cyclic physiological levels in a mammalian subject

(75) Inventors: Muriel Y. Ishikawa, Livermore, CA (US); Lowell L. Wood, JR., Bellevue, WA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 12/462,057

(22) Filed: Jul. 27, 2009

Related U.S. Application Data
(63) Continuation-in-part of application No. 12/220,708, filed on Jul. 24, 2008.
Application No. 12/220,708 is a continuation-in-part of application No. 12/220,704, filed on Jul. 24, 2008.
Application No. 12/220,704 is a continuation-in-part of application No. 12/220,707, filed on Jul. 24, 2008.
Application No. 12/220,707 is a continuation-in-part of application No. 12/455,272, filed on May 29, 2009.

(51) Int. Cl. A61K 39/395; A61K 38/00; A61K 31/7088; A61K 31/56

(52) U.S. Cl. 424/130.1; 514/2; 514/44 R; 514/169

(57) ABSTRACT

A method is described for treating or preventing a bone loss disease or a bone loss disorder in a mammalian subject or reducing the incidence of a bone loss disease or a bone loss disorder or alleviating the symptoms thereof. The method includes providing to the mammalian subject at least one treatment regimen including at least one follicle-stimulating hormone modulator configured to and in an amount sufficient to reduce bioactivity or bioavailability of follicle-stimulating hormone in the mammalian subject.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC §119(e) for provisional patent applications, for any and all parent, grandparent, great-grandparent, etc. applications of the Related Application(s)). All subject matter of the Related Applications and of any and all parent, grandparent, great-grandparent, etc. applications of the Related Applications is incorporated herein by reference to the extent such subject matter is not inconsistent herewith.

### RELATED APPLICATIONS

For purposes of the USPTO extra-statutory requirements, the present application constitutes a continuation-in-part of U.S. patent application Ser. No. 12/220,708, entitled METHOD, DEVICE, AND KIT FOR MAINTAINING PHYSIOLOGICAL LEVELS OF STEROID HORMONE IN A SUBJECT, naming Roderick A. Hyde, Muriel Y. Ishikawa, Dennis J. Rivet, Elizabeth A. Sweeney, Lowell L. Wood, Jr. and Victoria Y. H. Wood as inventors, filed 24 Jul. 2008, which is currently co-pending, or is an application of which a currently co-pending application is entitled to the benefit of the filing date.
For purposes of the USPTO extra-statutory requirements, the present application constitutes a continuation-in-part of U.S. patent application Ser. No. 12/220,704, entitled METHOD, DEVICE, AND KIT FOR MAINTAINING PHYSIOLOGICAL LEVELS OF STEROID HORMONE IN A SUBJECT, naming Roderick A. Hyde, Muriel Y. Ishikawa, Dennis J. Rivet, Elizabeth A. Sweeney, Lowell L. Wood, Jr. and Victoria Y. H. Wood as inventors, filed 24 Jul. 2008, which is currently co-pending, or is an application of which a currently co-pending application is entitled to the benefit of the filing date.
For purposes of the USPTO extra-statutory requirements, the present application constitutes a continuation-in-part of U.S. patent application Ser. No. 12/220,707, entitled SYSTEM AND DEVICE FOR MAINTAINING PHYSIOLOGICAL LEVELS OF STEROID HORMONE IN A SUBJECT, naming Roderick A. Hyde, Muriel Y. Ishikawa, Dennis J. Rivet, Elizabeth A. Sweeney, Lowell L. Wood, Jr. and Victoria Y. H. Wood as inventors, filed 24 Jul. 2008, which is currently co-pending, or is an application of which a currently co-pending application is entitled to the benefit of the filing date.
For purposes of the USPTO extra-statutory requirements, the present application constitutes a continuation-in-part of U.S. patent application Ser. No. 12/455,272, entitled METHOD FOR TREATING OR PREVENTING A CARDIOVASCULAR DISEASE OR CONDITION UTILIZING ESTROGEN RECEPTOR MODULATORS BASED ON APOE ALLELIC PROFILE OF A MAMMALIAN SUBJECT, naming Roderick A. Hyde, Muriel Y. Ishikawa, Eric C. Leuthardt, Dennis J. Rivet, Elizabeth A. Sweeney, Lowell L. Wood, Jr. and Victoria Y. H. Wood as inventors, filed 29 May 2009, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

### SUMMARY

A method is described herein for treating or preventing a bone loss disease or a bone loss disorder in a mammalian subject or reducing the incidence of a bone loss disease or a bone loss disorder or alleviating the symptoms thereof. The method includes providing to the mammalian subject at least one treatment regimen including at least one follicle-stimulating hormone modulator configured to and in an amount sufficient to reduce bioactivity or bioavailability of follicle-stimulating hormone in the mammalian subject. The at least one treatment regimen is configured to approximate the level of bioactive or bioavailable follicle-stimulating hormone to a target cyclic physiological pre-disease effective level in the mammalian subject. The at least one follicle-stimulating hormone modulator includes, but is not limited to, an inhibitor of follicle-stimulating hormone bioactivity, a follicle-stimulating hormone receptor antagonist, or an inhibitor of osteoclast activity. The at least one treatment regimen can be determined based on pre-disease cyclic levels of follicle-stimulating hormone in the mammalian subject and on current cyclic levels of follicle-stimulating hormone in the mammalian subject. The method which includes providing the at least one treatment regimen can further include providing a cyclic treatment regimen including at least one gonadotropin-releasing hormone modulator. The cyclic physiological pre-disease level can include a cyclic physiological premenopausal level in the mammalian subject.

The method including the at least one treatment regimen further includes providing replacement therapy including one or more steroid hormones or metabolites or modulators thereof. The at least one follicle-stimulating hormone modulator includes, but is not limited to, a small chemical molecule, polypeptide, nucleic acid, or antibody. In some aspects, the at least one treatment regimen is determined based on population data of physiological cyclic pre-disease levels of the one or more steroid hormones in one or more mammalian subjects. In further aspects, the at least one treatment regimen is determined based on pre-disease cyclic levels of steroid hormone in the mammalian subject and on current cyclic levels of steroid hormone in the mammalian subject. The at least one treatment regimen can be determined based on population data of physiological cyclic pre-disease levels of the one or more steroid hormones in one or more mammalian subjects. The at least one treatment regimen including the least one replacement therapy can be configured to increase levels of one or more of an estrogen or a progestogen, or metabolites or modulators thereof. The at least one treatment regimen can include replacement therapy with one or more of an estrogen or a progestogen. The at least one treatment regimen can be determined based on pre-disease cyclic levels of steroid hormone in the mammalian subject and on current cyclic levels of steroid hormone in the mammalian subject. The target cyclic physiological pre-disease level can include cyclic pulsatile levels of one or more of gonadotropin, follicle-stimulating hormone, luteinizing hormone, gonadotropin-releasing hormone, or steroid hormones. The target cyclic physiological pre-disease level of the follicle-stimulating hormone can be based on population data of cyclic physiological pre-disease levels of the one or more of gonadotropin, follicle-stimulating hormone, luteinizing hormone, gonadotropin-releasing hormone, or steroid hormones in one or more mammalian subjects. The at least one treatment regimen can be configured to maintain the subject's one or more steroid hormones or metabolites or modulators thereof at substantially physiological pre-disease levels.

The method described herein for treating or preventing a bone loss disease or a bone loss disorder in a mammalian subject can further include determining the one or more gonadotropin levels or the one or more steroid hormones levels in the subject during a treatment period. In some aspects, the treatment period can include a time period preceding treatment with the at least one follicle-stimulating hormone modulator. The treatment period can include a time period during treatment with the at least one follicle-stimulating hormone modulator. In further aspects, the determining of the one or more gonadotropin levels or the one or more steroid hormones levels can occur at multiple time points during the treatment period.

In the method described herein, the at least one treatment regimen can be determined based at least in part on one or more of a time-history of gonadotropin levels or serum steroid hormone levels in the subject, on inferred peak values or minimal values of serum gonadotropin levels or serum steroid hormone levels in the subject, on age of the subject, or on categorization relative to profiles of patient populations. The at least one treatment regimen can be determined based at least in part on Fourier analysis of the cyclic gonadotropin levels or cyclic steroid hormone levels in the subject, or on harmonic analysis of the cyclic gonadotropin levels or the cyclic steroid hormone levels in the subject. In some aspects, the at least one treatment regimen can be determined based at least in part on scaled values of the gonadotropin levels or the steroid hormone levels prior to the disease diagnosis in the subject. In further aspects, the at least one treatment regimen can be determined based at least in part on the scaled value approximately equal to one. The at least one treatment regimen can be determined based at least in part on the scaled value dependent on age of the subject.

In some aspects, the bone loss disease or the bone loss disorder can include osteoporosis, osteomyelitis, Paget's disease, periodontitis, hypercalcemia, osteonecrosis, osteosarcoma, osteolyic metastases, familial expansile osteolysis, prosthetic loosening, periprostetic osteolysis, juxtaarticular bone destruction in rheumatoid arthritis, or cleiodocranial dysplasia (CCD). The at least one follicle-stimulating hormone modulator can include a gonadotropin releasing hormone antagonist, FSH inhibitor, FSH synthesis inhibitor, FSH secretion inhibitor, or FSH receptor antagonist. The gonadotropin releasing hormone antagonist can include synthetic decapeptide, synthetic nonapeptide, ganirelix, cetrorelix, degarelix, or abarelix. The gonadotropin releasing hormone antagonist can include NBI-56418, tetrahydroquinolines, diketopiperazines, sulphonamides, thiazolidinones, sulphonic acids, azo compounds, pyrrolobenzodiazepines, or oracyltryptophanols. The FSH inhibitor can include inhibin A, inhibin B, analogs or mimetics of inhibin A or inhibin B, FSH analogs or mimetics, FSH-binding antibodies, activin antagonist or inhibitor, activin-binding glycoprotein, follistatin, or FLRG protein. The FSH synthesis inhibitor or FSH secretion inhibitor can include antisense oligonucleotide; siRNA, shRNA, or double stranded RNA. The FSH receptor antagonist can include soluble FSH receptor, or antibodies to FSH receptor.

A method is described herein for preventing a bone loss disease or a bone loss disorder in a mammalian subject that includes providing to the mammalian subject at least one treatment regimen including at least one follicle-stimulating hormone modulator configured to and in an amount sufficient to reduce or maintain bioactivity or bioavailability of follicle-stimulating hormone in the mammalian subject, and to approximate the level of bioactive or bioavailable follicle-stimulating hormone to a target cyclic physiological disease-free effective level in the mammalian subject. The at least one follicle-stimulating hormone modulator includes, but is not limited to, an inhibitor of follicle-stimulating hormone bioactivity, a follicle-stimulating hormone receptor antagonist, or an inhibitor of osteoclast activity. The at least one treatment regimen can be determined based on pre-disease cyclic levels of follicle-stimulating hormone in the mammalian subject and on current cyclic levels of follicle-stimulating hormone in the mammalian subject. The method which includes providing the at least one treatment regimen can further include providing a cyclic treatment regimen including at least one gonadotropin-releasing hormone modulator. The cyclic physiological pre-disease level can include a cyclic physiological premenopausal level in the mammalian subject.

The method including the at least one treatment regimen further includes providing replacement therapy including one or more steroid hormones or metabolites or modulators thereof. The at least one follicle-stimulating hormone modulator includes, but is not limited to, a small chemical molecule, polypeptide, nucleic acid, or antibody. In some aspects, the at least one treatment regimen is determined based on population data of physiological cyclic pre-disease levels of the one or more steroid hormones in one or more mammalian subjects. In further aspects, the at least one treatment regimen is determined based on pre-disease cyclic levels of steroid hormone in the mammalian subject and on current cyclic levels of steroid hormone in the mammalian subject. The at least one treatment regimen can be determined based on population data of physiological cyclic pre-disease levels of the one or more steroid hormones in one or more mammalian subjects. The at least one treatment regimen including the least one replacement therapy can be configured to increase levels of one or more of an estrogen or a progestogen, or metabolites or modulators thereof. The at least one treatment regimen can include replacement therapy with one or more of an estrogen or a progestogen. The at least one treatment regimen can be determined based on pre-disease cyclic levels of steroid hormone in the mammalian subject and on current cyclic levels of steroid hormone in the mammalian subject. The target cyclic physiological pre-disease level can include cyclic pulsatile levels of one or more of gonadotropin, follicle-stimulating hormone, luteinizing hormone, gonadotropin-releasing hormone, or steroid hormones. The target cyclic physiological pre-disease level of the follicle-stimulating hormone can be based on population data of cyclic physiological pre-disease levels of the one or more of gonadotropin, follicle-stimulating hormone, luteinizing hormone, gonadotropin-releasing hormone, or steroid hormones in one or more mammalian subjects. The at least one treatment regimen can be configured to maintain the subject's one or more steroid hormones or metabolites or modulators thereof at substantially physiological pre-disease levels.

The method described herein for treating or preventing a bone loss disease or a bone loss disorder in a mammalian subject can further include determining the one or more gonadotropin levels or the one or more steroid hormones levels in the subject during a treatment period. In some aspects, the treatment period can include a time period preceding treatment with the at least one follicle-stimulating hormone modulator. The treatment period can include a time period during treatment with the at least one follicle-stimulating hormone modulator. In further aspects, the determining of the one or more gonadotropin levels or the one or more steroid hormones levels can occur at multiple time points during the treatment period.

In the method described herein, the at least one treatment regimen can be determined based at least in part on one or more of a time-history of gonadotropin levels or serum steroid hormone levels in the subject, on inferred peak values or minimal values of serum gonadotropin levels or serum steroid hormone levels in the subject, on age of the subject, or on categorization relative to profiles of patient populations. The at least one treatment regimen can be determined based at least in part on Fourier analysis of the cyclic gonadotropin levels or cyclic steroid hormone levels in the subject, or on harmonic analysis of the cyclic gonadotropin levels or the cyclic steroid hormone levels in the subject. In some aspects, the at least one treatment regimen can be determined based at least in part on scaled values of the gonadotropin levels or the steroid hormone levels prior to the disease diagnosis in the subject. In further aspects, the at least one treatment regimen can be determined based at least in part on the scaled value approximately equal to one. The at least one treatment regimen can be determined based at least in part on the scaled value dependent on age of the subject. The at least one follicle-stimulating hormone modulator can include a gonadotropin releasing hormone antagonist, FSH inhibitor, FSH synthesis inhibitor, FSH secretion inhibitor, or FSH receptor antagonist.

A method is described herein for maintaining a substantially physiological cyclic pre-menopausal level of follicle-stimulating hormone in a female subject that includes providing to the mammalian subject at least one treatment regimen including at least one follicle-stimulating hormone modulator configured to and in an amount sufficient to reduce bioactivity or bioavailability of follicle-stimulating hormone in the female subject, and to approximate the level of bioactive or bioavailable follicle-stimulating hormone to a target cyclic physiological pre-menopausal effective level in the female subject. The at least one follicle-stimulating hormone modulator includes, but is not limited to, an inhibitor of follicle-stimulating hormone bioactivity, a follicle-stimulating hormone receptor antagonist, or an inhibitor of osteoclast activity. The at least one treatment regimen can be determined based on pre-disease cyclic levels of follicle-stimulating hormone in the mammalian subject and on current cyclic levels of follicle-stimulating hormone in the mammalian subject. The method which includes providing the at least one treatment regimen can further include providing a cyclic treatment regimen including at least one gonadotropin-releasing hormone modulator. The cyclic physiological pre-disease level can include a cyclic physiological premenopausal level in the mammalian subject.

The method including the at least one treatment regimen further includes providing replacement therapy including one or more steroid hormones or metabolites or modulators thereof. The at least one follicle-stimulating hormone modulator includes, but is not limited to, a small chemical molecule, polypeptide, nucleic acid, or antibody. In some aspects, the at least one treatment regimen is determined based on population data of physiological cyclic pre-disease levels of the one or more steroid hormones in one or more mammalian subjects. In further aspects, the at least one treatment regimen is determined based on pre-disease cyclic levels of steroid hormone in the mammalian subject and on current cyclic levels of steroid hormone in the mammalian subject. The at least one treatment regimen can be determined based on population data of physiological cyclic pre-disease levels of the one or more steroid hormones in one or more mammalian subjects. The at least one treatment regimen including the least one replacement therapy can be configured to increase levels of one or more of an estrogen or a progestogen, or metabolites or modulators thereof. The at least one treatment regimen can include replacement therapy with one or more of an estrogen or a progestogen. The at least one treatment regimen can be determined based on pre-disease cyclic levels of steroid hormone in the mammalian subject and on current cyclic levels of steroid hormone in the mammalian subject. The target cyclic physiological pre-disease level can include cyclic pulsatile levels of one or more of gonadotropin, follicle-stimulating hormone, luteinizing hormone, gonadotropin-releasing hormone, or steroid hormones. The target cyclic physiological pre-disease level of the follicle-stimulating hormone can be based on population data of cyclic physiological pre-disease levels of the one or more of gonadotropin, follicle-stimulating hormone, luteinizing hormone, gonadotropin-releasing hormone, or steroid hormones in one or more mammalian subjects. The at least one treatment regimen can be configured to maintain the subject's one or more steroid hormones or metabolites or modulators thereof at substantially physiological pre-disease levels.

The method described herein for treating or preventing a bone loss disease or a bone loss disorder in a mammalian subject can further include determining the one or more gonadotropin levels or the one or more steroid hormones levels in the subject during a treatment period. In some aspects, the treatment period can include a time period preceding treatment with the at least one follicle-stimulating hormone modulator. The treatment period can include a time period during treatment with the at least one follicle-stimulating hormone modulator. In further aspects, the determining of the one or more gonadotropin levels or the one or more steroid hormones levels can occur at multiple time points during the treatment period.

In the method described herein, the at least one treatment regimen can be determined based at least in part on one or more of a time-history of gonadotropin levels or serum steroid hormone levels in the subject, on inferred peak values or minimal values of serum gonadotropin levels or serum steroid hormone levels in the subject, on age of the subject, or on categorization relative to profiles of patient populations. The at least one treatment regimen can be determined based at least in part on Fourier analysis of the cyclic gonadotropin levels or cyclic steroid hormone levels in the subject, or on harmonic analysis of the cyclic gonadotropin levels or the cyclic steroid hormone levels in the subject. In some aspects, the at least one treatment regimen can be determined based at least in part on scaled values of the gonadotropin levels or the steroid hormone levels prior to the disease diagnosis in the subject. In further aspects, the at least one treatment regimen can be determined based at least in part on the scaled value approximately equal to one. The at least one treatment regimen can be determined based at least in part on the scaled value dependent on age of the subject. The at least one follicle-stimulating hormone modulator can include a gonadotropin releasing hormone antagonist, FSH inhibitor, FSH synthesis inhibitor, FSH secretion inhibitor, or FSH receptor antagonist.

A system is described herein that includes a sensor configured to detect one or more hormones in one or more tissues of the mammalian subject, and a controller in communication with the sensor, wherein the controller is configured to provide at least one treatment regimen including at least one follicle-stimulating hormone modulator configured to and in an amount sufficient to reduce bioactivity or bioavailability of follicle-stimulating hormone in the mammalian subject, and to approximate the level of bioactive or bioavailable follicle-stimulating hormone to a target cyclic physiological pre-disease effective level in the mammalian subject. The one or more hormones can include, but is not limited to, follicle-stimulating hormone, luteinizing hormone, or steroid hormone. The steroid hormone can include, but is not limited to, estrogen, progestogen, or testosterone. The at least one follicle-stimulating hormone modulator can include an inhibitor of follicle-stimulating hormone bioactivity. The at least one follicle-stimulating hormone modulator can include a follicle-stimulating hormone receptor antagonist. The at least one follicle-stimulating hormone modulator can include an inhibitor of osteoclast activity. The at least one follicle-stimulating hormone modulator can include, but is not limited to, a small chemical molecule, polypeptide, nucleic acid, or antibody.

The system including at least one treatment regimen can further include providing replacement therapy including one or more steroid hormones or metabolites or modulators thereof. The at least one treatment regimen can be determined based on population data of physiological cyclic pre-disease levels of the one or more steroid hormones in one or more mammalian subjects. The at least one treatment regimen including the least one replacement therapy can be configured to increase levels of one or more of an estrogen or a progestogen, or metabolites or modulators thereof. The at least one treatment regimen can include replacement therapy with one or more of an estrogen or a progestogen. The at least one treatment regimen can be determined based on pre-disease cyclic levels of steroid hormone in the mammalian subject and on current cyclic levels of steroid hormone in the mammalian subject. The at least one treatment regimen can be determined based on pre-disease cyclic levels of follicle-stimulating hormone in the mammalian subject and on current cyclic levels of follicle-stimulating hormone in the mammalian subject. The target cyclic physiological pre-disease level can include cyclic pulsatile levels of one or more of gonadotropin, follicle-stimulating hormone, luteinizing hormone, gonadotropin-releasing hormone, or steroid hormones. The target cyclic physiological pre-disease level of the follicle-stimulating hormone can be based on population data of cyclic physiological pre-disease levels of one or more of gonadotropin, follicle-stimulating hormone, luteinizing hormone, gonadotropin-releasing hormone, or steroid hormones in one or more mammalian subjects. The system as described herein wherein providing the at least one treatment regimen can further include providing a cyclic treatment regimen including one or more of at least one gonadotropin, or at least one gonadotropin-releasing hormone modulator. The system as described herein wherein the at least one follicle-stimulating hormone modulator can include, but is not limited to, a gonadotropin releasing hormone antagonist, FSH inhibitor, FSH synthesis inhibitor, FSH secretion inhibitor, or FSH receptor antagonist.

A method is described herein for treating a bone loss disease or a bone loss disorder in a mammalian subject that includes providing a system including a sensor configured to detect one or more hormones in one or more tissues of the mammalian subject; and a controller in communication with the sensor, wherein the controller is configured to provide to the mammalian subject at least one treatment regimen including at least one follicle-stimulating hormone modulator configured to and in an amount sufficient to reduce bioactivity or bioavailability of follicle-stimulating hormone in the mammalian subject, and to approximate the level of bioactive or bioavailable follicle-stimulating hormone to a target cyclic physiological pre-disease effective level in the mammalian subject. The one or more hormones can include, but is not limited to, follicle-stimulating hormone, luteinizing hormone, or steroid hormone. The steroid hormone can include, but is not limited to, estrogen, progestogen, or testosterone.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 depicts a diagrammatic view of one aspect of an exemplary embodiment of a method for treating or preventing a bone loss disease or a bone loss disorder in a mammalian subject in need thereof.

FIG. 2 depicts a logic flowchart of a method for treating or preventing a bone loss disease or a bone loss disorder in a mammalian subject in need thereof.

FIG. 3 depicts some aspects of a system that may serve as an illustrative environment for subject matter technologies.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The present application uses formal outline headings for clarity of presentation. However, it is to be understood that the outline headings are for presentation purposes, and that different types of subject matter may be discussed throughout the application (e.g., method(s) may be described under composition heading(s) and/or kit headings; and/or descriptions of single topics may span two or more topic headings). Hence, the use of the formal outline headings is not intended to be in any way limiting.

A method is described herein for treating or preventing a bone loss disease or a bone loss disorder in a mammalian subject or reducing the incidence of a bone loss disease or a bone loss disorder or alleviating the symptoms thereof. The method includes providing to the mammalian subject at least one treatment regimen including at least one follicle-stimulating hormone modulator configured to and in an amount sufficient to reduce bioactivity or bioavailability of follicle-stimulating hormone in the mammalian subject. The at least one treatment regimen is configured to approximate the level of bioactive or bioavailable follicle-stimulating hormone to a target cyclic physiological pre-disease effective level in the mammalian subject. The at least one follicle-stimulating hormone modulator includes, but is not limited to, an inhibitor of follicle-stimulating hormone bioactivity, a follicle-stimulating hormone receptor antagonist, or an inhibitor of osteoclast activity. The at least one treatment regimen further includes providing replacement therapy including one or more steroid hormones or metabolites or modulators thereof. In some aspects, the at least one treatment regimen is determined based on population data of physiological cyclic pre-disease levels of the one or more steroid hormones. In further aspects, the at least one treatment regimen is determined based on pre-disease cyclic levels of steroid hormone in the mammalian subject and on current cyclic levels of steroid hormone in the mammalian subject.

Hormone replacement or supplemental therapy has been used for some time to relieve symptoms of menopause or to provide protection from disorders such as osteoporosis. However, early and more recent studies have offered evidence that treatment with exogenous hormones carries risks, and limits have been suggested for treatments, including those on dosages and formulations. While incorporating these limitations, therapies can be designed based on population data, or can be based upon individualized treatment regimens developed from individual medical history data on hormonal levels. Methods described herein include treatment regimens including FSH modulators, and optionally, steroid hormones, that regulate hormone levels to approach a target cyclic physiological pre-disease effective level of FSH, LH, and steroid hormones. The treatment regimen is configured to achieve reduced bioactivity or bioavailability of FSH and LH and a cyclic spike of bioactivity or bioavailability of FSH and LH, and to approximate the level of bioactive or bioavailable follicle-stimulating hormone to a target cyclic physiological pre-disease levels of FSH and LH in the mammalian subject. Steroid hormone levels are elevated to pre-disease physiological levels of steroid hormone.

A method is described herein for treating or preventing osteoporosis in a mammalian subject. Osteoporosis affects nearly 45 million women worldwide with fracture rates that far exceed the combined incidence of breast cancer, stroke, and heart attacks. The disease results from a disruption of the fine balance between osteoblastic bone formation and osteoclastic bone resorption. After menopause, resorption significantly exceeds formation, and this imbalance results in net bone loss. Estrogen replacement slows postmenopausal bone loss and reduces the risk of fracture. Postmenopausal osteoporosis, a global public health problem, has for decades been attributed solely to declining estrogen levels. FSH levels rise sharply in parallel, and a direct effect of FSH on bone mass density (BMD) has been explored.

Studies have suggested that the pathophysiology of bone loss during early menopause and in hypogonadism, which has been attributed solely to declining sex hormone levels, may result at least in significant part from elevated circulating FSH. Reduced FSH levels and increased BMD correlate well following estrogen replacement therapy. Studies concluded that a high circulating FSH causes postmenopausal and hypogonadal osteoporosis. See, e.g., Cauley et al., JAMA 290: 1729-1738, 2003; Lindsay, R., Endocrine 24: 223-230, 2004; Sun, et al. Cell 125: 247-260, 2006; U.S. Patent Application 2008/0069811, which are incorporated herein by reference.

The method described herein for treating osteoporosis in a mammalian subject includes providing to the mammalian subject at least one treatment regimen including at least one FSH modulator configured to and in an amount sufficient to reduce bioactivity or bioavailability of FSH in the mammalian subject. The at least one treatment regimen further includes providing replacement therapy including one or more steroid hormones or metabolites or modulators thereof. The at least one treatment regimen is configured to approximate the level of bioactive or bioavailable follicle-stimulating hormone to a target cyclic physiological pre-disease effective level of FSH, LH, and one or more steroid hormones in the mammalian subject. The target cyclic physiological pre-disease effective level of the FSH includes reduced bioactivity or bioavailability of FSH and a cyclic spike of bioactivity or bioavailability of FSH during a 28-day cycle. The target cyclic physiological pre-disease effective level of the FSH is based on population data or based on individual patient data derived from one or more pre-disease mammalian subjects or one or more premenopausal mammalian subjects.

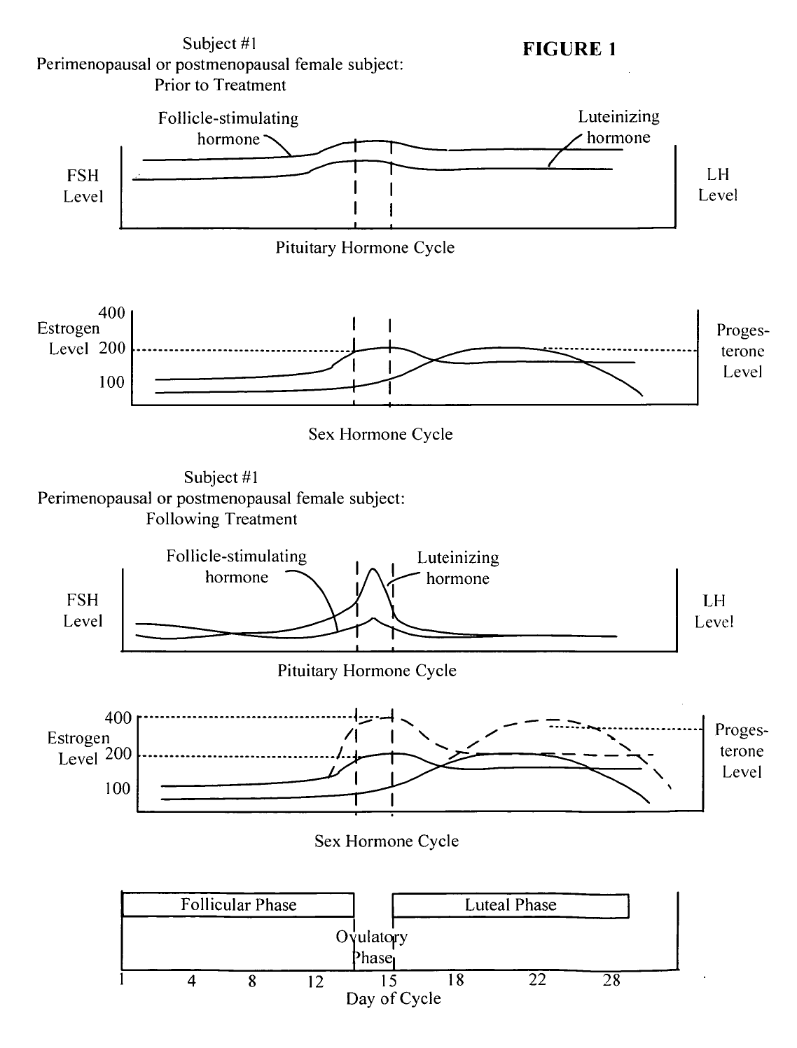

FIG. 1 depicts a diagrammatic view of an aspect of the methods and systems as described herein. The methods described herein for treating a bone loss disease or a bone loss disorder in a mammalian subject in need thereof are based on population hormone levels or based on individualized hormone levels for a mammalian subject #1. Female subject #1 has perimenopausal or postmenopausal cyclic levels of steroid hormones, e.g., follicle stimulating hormone (FSH) is elevated, luteinizing hormone (LH) is elevated, estrogen and progesterone are reduced when measured over a time period of 28 days prior to treatment. See solid lines on graph in FIG. 1, “Subject #1, Perimenopausal or postmenopausal female subject: Prior to treatment”. Female subject #1 in a perimenopausal or postmenopausal condition following treatment with an FSH modulator, and optionally one or more steroid hormones or metabolites or modulators thereof, has estrogen and progesterone levels elevated to a target cyclic physiological pre-disease levels. FSH levels and LH levels are reduced to a target cyclic physiological pre-disease effective level including a cyclic spike in FSH and LH levels. See solid lines on graph in FIG. 1; “Subject #1, Perimenopausal or postmenopausal female subject: Following treatment”. A method is described herein for treating a bone loss disease or a bone loss disorder in a mammalian subject. The method includes providing to the mammalian subject at least one treatment regimen including at least one follicle-stimulating hormone modulator configured to and in an amount sufficient to reduce bioactivity or bioavailability of follicle-stimulating hormone in the mammalian subject, and to approximate the level of bioactive or bioavailable follicle-stimulating hormone to a target cyclic physiological pre-disease effective level in the mammalian subject. The at least one treatment regimen further includes providing replacement therapy including one or more steroid hormones or metabolites or modulators thereof. The at least one treatment regimen is determined based on population data of physiological cyclic pre-disease levels of the one or more steroid hormones in one or more mammalian subjects. See dashed lines on graph in FIG. 1; “Subject #1, Perimenopausal or postmenopausal female subject: Following treatment”.

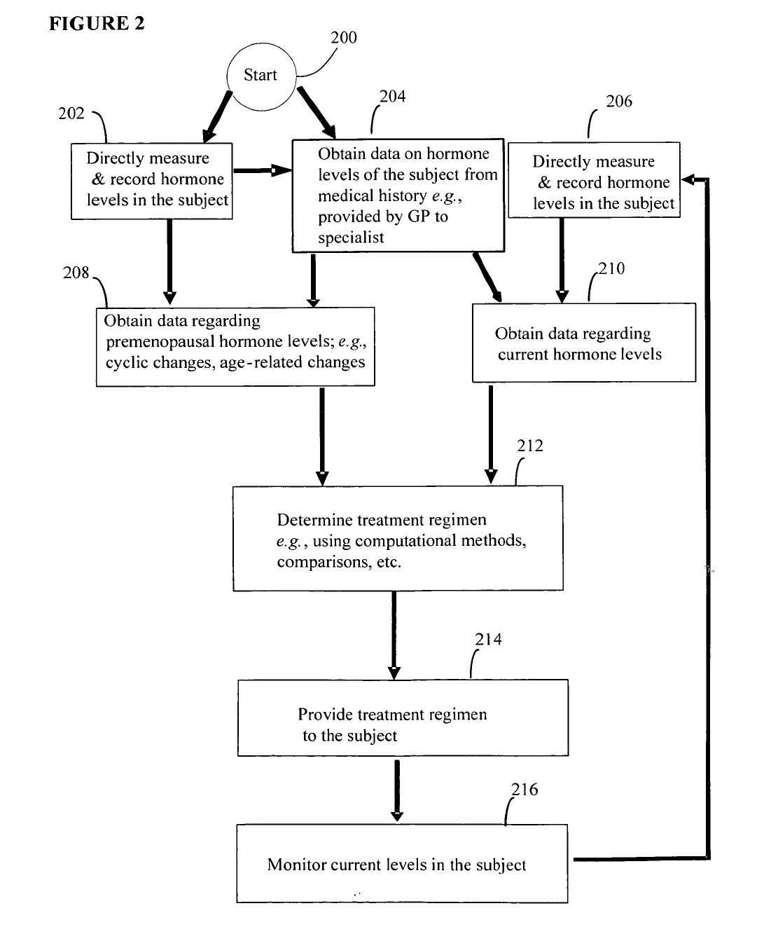

FIG. 2 depicts a high-level logic flowchart of a process. Method step 200 shows the start of the process. Method step 202 depicts directly measuring and recording hormone levels in the subject. Method step 204 depicts obtaining data regarding hormone levels from a medical history of the subject. Method step 208 depicts obtaining data regarding premenopausal hormone levels in the subject from method steps 202 and/or 204. This data can reflect, e.g., cyclic hormonal changes or age-related hormonal changes in the subject. Method step 206 depicts directly measuring and recording hormone levels in the subject wherein the subject can be premenopausal, perimenopausal, early or late menopausal, or post menopausal. Method step 210 depicts obtaining data regarding current hormone levels from method steps 204 and/or 206. Method step 212 depicts determining a treatment regimen using methods e.g., including, but not limited to, computational methods or comparison methods. Method step 214 depicts providing at least one treatment regimen including replacement therapy for the one or more steroid hormones or metabolites or modulators thereof, to the subject. Method step 216 depicts monitoring current hormone levels during treatment of the subject. Method step 206 depicts directly measuring and recording hormone levels, e.g., during treatment of the subject. Method step 210 depicts obtaining data regarding current hormone levels. The data regarding current hormone levels is obtained from directly measuring and recording 206 current hormone levels during treatment of the subject and/or from obtaining data 204 on hormone levels from a medical history of the subject. The data is used to determine the proper treatment regimen 212 and alter or adjust the treatment regimen as needed, and providing the treatment regimen 214 to the subject. In an embodiment, method steps 202, 204, 206, 208, 210, 212, 214, 216, 218, 220, and/or 222 can include accepting input related to, for example, directly measuring and recording hormone levels in the subject, obtaining data on hormone levels from medical history of the subject, determining a treatment regimen, providing a treatment regimen and monitoring current hormone levels during treatment of the subject.

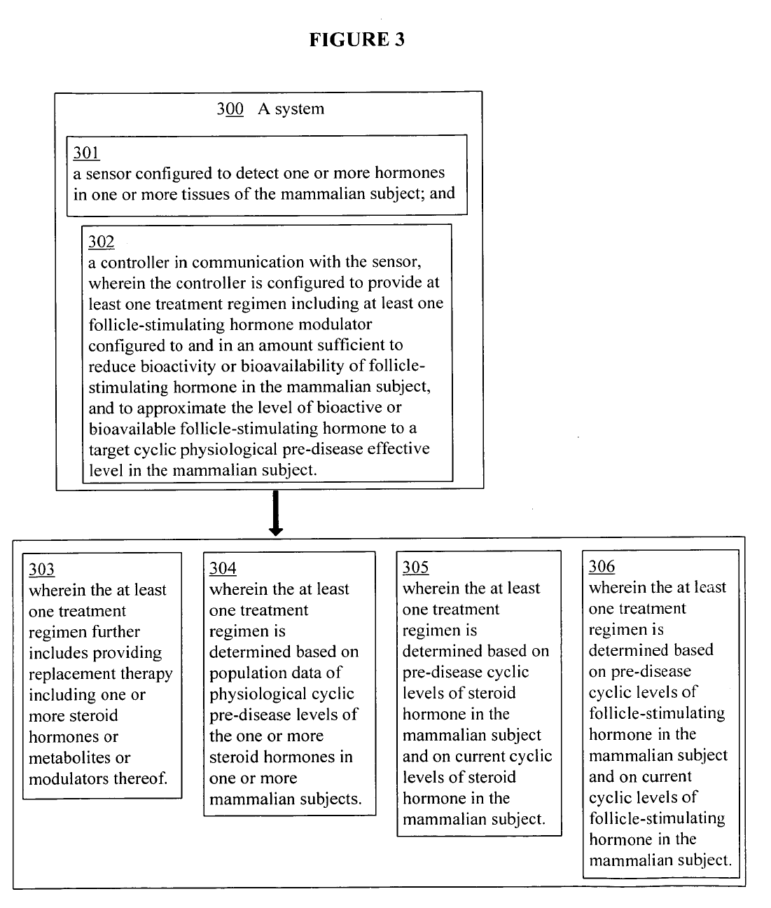

FIG. 3 depicts some aspects of a system that may serve as an illustrative environment for subject matter technologies. The system 300 includes a sensor 301 configured to detect one or more hormones in one or more tissues of the mammalian subject; and a controller 302 in communication with the sensor, wherein the controller is configured to provide at least one treatment regimen including at least one follicle-stimulating hormone modulator configured to and in an amount sufficient to reduce bioactivity or bioavailability of follicle-stimulating hormone in the mammalian subject, and to approximate the level of bioactive or bioavailable follicle-stimulating hormone to a target cyclic physiological pre-disease effective level in the mammalian subject.

### Follicle Stimulating Hormone and Follicle Stimulating Hormone Receptor

A method is described for treating a bone loss disease or a bone loss disorder in a mammalian subject that includes providing to the mammalian subject at least one treatment regimen including at least one follicle-stimulating hormone modulator configured to and in an amount sufficient to reduce bioactivity or bioavailability of follicle-stimulating hormone in the mammalian subject, and to approximate the level of bioactive or bioavailable follicle-stimulating hormone to a target cyclic physiological pre-disease effective level in the mammalian subject. Follicle stimulating hormone (FSH) is a gonadotrophin hormone synthesized and secreted by gonadotropes in the anterior pituitary gland. FSH is defined in molecular terms as a heterodimeric glycoprotein hormone consisting of two noncovalently linked subunits designated alpha and beta. In the case of human FSH, the subunits are 92 amino acids and 111 amino acids, respectively, and each has two N-linked glycosylation sites that are essential for FSH bioactivity. FSH has several biological functions in mammals. In males, for example, FSH, in combination with testosterone is required for the initiation and maintenance of qualitatively and quantitatively normal spermatogenesis. In females, FSH is necessary for selection and growth of ovarian follicles and for the production of estrogens from androgen substrate.

FSH is part of the hypothalamo-pituitary-ovarian axis, a classic endocrine closed loop biofeedback system, in which the gonadotrophins (e.g., follicle-stimulating hormone (FSH) and luteinizing hormone (LH)). stimulate ovarian hormone production (e.g., estrogen), which in turn exerts a negative feedback effect on the gonadotrophins, to maintain a regulated system. Gonadotrophins include hormones produced by the pituitary gland that regulate the gonads, such as follicle-stimulating hormone (FSH) and luteinizing hormone (LH). In women gonadotropins regulate the development of the ovaries and eggs. In men gonadotropins regulate the development of testes. The secretion of FSH is stimulated by gonadotropin releasing hormone (GnRH). At the beginning of each menstrual cycle, FSH stimulates the growth and recruitment of immature ovarian follicles in the ovary. After 5-6 days, one dominant follicle begins to develop more rapidly. The outer theca and inner granulosa cells of the follicle multiply and under the influence of FSH and LH begin to secrete estrogen and the peptide hormone inhibin. The increase in serum estrogen levels inhibits GnRH which in turn leads to a decrease in FSH production. Similarly, inhibin inhibits the synthesis and secretion of FSH. Estrogens and inhibin secreted by the ovary inhibit the activity of FSH leading to regression of the smaller, less mature follicles. The estrogen levels peak just before midcycle, and the granulosa cells begin to secrete progesterone. These relative changes in estrogen and progesterone stimulate a brief surge in FSH and LH release that precedes and initiates ovulation.

The cohort of small antral follicles in the ovaries is normally sufficient in number to produce enough estrogen and inhibin to lower FSH serum levels at appropriate times during the menstrual cycle. However, as a woman nears perimenopause the number of small antral follicles recruited in each cycle diminishes and consequently insufficient estrogen and inhibin is produced to appropriately modulate the levels of FSH. The decline in estrogen and inhibin are concomitant with the gradual deterioration of the ovaries as a women progresses through menopause and into postmenopause. As a result, the negative feedback that normally modulates FSH secretion is gone, leading to significantly increased FSH serum levels. For example, in women over 35, FSH levels rise gradually at the beginning of the follicular phase. This rise becomes more marked after the age of 45 and at the onset of perimenopause (changes in menstrual cycles, irregular cycles, menopausal symptoms). The rise continues until after the menopause. LH levels also rise at the menopause but to a much lesser extent than FSH levels.

The circulating levels of FSH in a human female fluctuates over the course of her life. The pre-puberty basal levels of FSH range from about 0.2 U/liter to about 2.0 U/liter and increase to about 4.0 U/liter to about 5.0 U/liter during puberty. During the mid-reproductive years, the FSH levels fluctuate cyclically with the normal menstrual cycle. Circulating FSH levels begin to increase about 4 days pre-menstrually, reach a mid-follicular phase peak, gradually fall prior to the mid-cycle surge and then decline to low levels during the luteal phase. For example, the levels of FSH during the follicular phase of the cycle range from about 2.5 U/liter to about 10.2 U/liter. At the midcycle peak, the FSH levels rise to a range from about 3.4 U/liter to about 33.4 U/liter. During the luteal phase, the FSH levels fall and range from about 1.5 U/liter to about 9.1 U/liter. As the human female ages, the FSH levels begin to increase. During early menopausal transition or perimenopause, the FSH levels increase to an average of about 10-22 U/liter. The FSH levels continue to rise as the human female reaches late perimenopause and post-menopause to on average ranging from about 23 U/liter to greater than 100 U/liter. See, e.g., Belgorosky, et al., J. Clin. Endocrinol. Metab. 88:5127-5131, 2003; Prior Endocr. Rev. 19:397-428, 1998; Chada, et al., Physiol. Res. 52:341-346, 2003, Burger, et al., Hum. Reprod. Update 13:559-565, 2007; which are incorporated herein by reference.

The increase in FSH during post-menopause can be attributed to a lack of negative feed back from estrogen and inhibin that are no longer being secreted by the ovaries. Increased FSH may contribute to the decline in bone health associated with postmenopausal women. Follicle stimulating hormone (FSH) acts by binding to specific receptors localized primarily in Sertoli cells of the testis and in granulosa cells of the ovary. The FSH receptor belongs to the family of G protein-coupled receptors (GPCR) which are complex membrane-associated receptors characterized by seven-transmembrane spanning domains. The intracellular portion of the FSH receptor is coupled to a G-protein S and adenylate cyclase and upon receptor activation by FSH with the extracellular domain, initiates a cascade of cAMP-protein kinase A mediated signaling events that ultimately leads to the specific biological effects of FSH. See, e.g., Simoni, et al., Endocr. Rev. 18: 739-773, 1997, which is incorporated herein by reference.

The FSH receptor has also been localized to cellular components of bone. More specifically, FSH receptors coupled to G_{i2α} have been detected in osteoclasts, the cells in bone responsible for bone resorption. Treatment of osteoclast precursor cells with FSH results in increased osteoclastogenesis while treatment of differentiated osteoclasts with FSH results in increased resorption. These results suggest that the increased FSH observed in perimenopausal and postmenopausal women may contribute to the increased risk of bone loss and osteoporosis in this population. See, e.g., Sun, et al. Cell 125: 247-260, 2006; US Patent Application 2008/0069811, which are incorporated herein by reference.

### Osteoporosis

A method is described for treating a bone loss disease or a bone loss disorder. In some aspects, the bone loss disease or bone loss disorder is osteoporosis. The method includes providing to the mammalian subject at least one treatment regimen including at least one follicle-stimulating hormone modulator configured to and in an amount sufficient to reduce bioactivity or bioavailability of follicle-stimulating hormone in the mammalian subject, and to approximate the level of bioactive or bioavailable follicle-stimulating hormone to a target cyclic physiological pre-disease effective level in the mammalian subject. Osteoporosis, which means “porous bones”, is a disease characterized by low bone mass and structural deterioration of bone tissue, leading to bone fragility and an increased susceptibility to fractures, especially of the hip, spine, and wrist. Osteoporosis occurs primarily as a result of normal aging, but can arise as a result of impaired development of peak bone mass (e.g. due to delayed puberty or poor nutrition) or excessive bone loss during adulthood (e.g. due to estrogen deficiency in women, poor nutrition, or corticosteroid use). In healthy young adults, bone formation and bone resorption are balanced, resulting in no net increase or decrease in skeletal mass. With advancing age, men and women experience an imbalance in bone remodeling in which resorption is slightly greater than formation, resulting in a continuous net loss of bone mass with time. If this imbalance persists, bone mass can decline until the skeleton is insufficient to withstand normal mechanical stresses, and it become abnormally susceptible to fractures. The most common form of osteoporosis occurs in postmenopausal women and is the result of estrogen deficiency. Rapid bone loss accompanies the decline of estrogen levels at the onset of menopause or as a result of surgical removal of the ovaries (oophorectomy). Rapid bone loss occurs as a result of the combined effects of imbalance in bone remodeling and an increase in bone turnover.

The bone loss disease or bone loss disorder, e.g., osteoporosis, is operationally defined based on bone mineral density (BMD) assessment. In mammalian subjects with osteoporosis, the bone mineral density (BMD) is reduced, the bone microarchitecture is disrupted, and the amount and variety of non-collagenous proteins in bone is altered. Osteoporosis is defined by the World Health Organization (WHO) as a bone mineral density that lies 2.5 standard deviations or more below the average value for healthy women; T score <−2.5. See, e.g., World Health Organization, “WHO Scientific Group on the Assessment of Osteoporosis at Primary Health Care Level” Summary Meeting Report, Brussels, Belgium, 5-7 May 2004, which is incorporated herein by reference. T-scores ranging from about −1.0 or higher are considered normal. T-scores ranging from less than −1.0 and greater than −2.5 are indicative of osteopenia, a possible precursor to osteoporosis. T-scores of −2.5 or lower are indicative of osteoporosis. There is a strong inverse relationship between BMD and the risk of fracture, with a 2-3 fold increase in fracture incidence for each standard deviation reduction in the BMD. See, e.g., Newton, et al. Q. J. Med. 99:231-236, 2006, which is incorporated herein by reference.

Bone mineral density can be measured in a mammalian subject using any of a number of noninvasive imaging techniques including but not limited to x-ray absorptiometry, computed tomography, ultrasound, and single and dual absorptiometry. Specific examples include dual energy x-ray absorptiometry (DXA) commonly measured at the hip, spine and/or whole body; peripheral dual energy x-ray absorptiometry (pDXA) commonly measured at the wrist, heel and/or finger; single energy x-ray absorptiometry (SXA) commonly measured at the wrist and/or heel; quantitative ultrasound (QUS) commonly measured at the heel, shin bone, and/or kneecap; quantitative computed tomography (QCT) commonly measured at the spine and/or other sites; peripheral quantitative computed tomography (pQCT) commonly measured at the wrist; radiographic absorptiometry (RA). These examples commonly use x-ray of the hand in combination with a small metal wedge; dual photon absorptiometry (DPA) commonly measured at the spine, hip and/or whole body; single photon absorptiometry (SPA) commonly measured at the wrist.

The bone mineral density of the mammalian subject as measured by one or more methods described herein is compared with one or more standards such as, for example, age matched standards and/or young normal standards. The age matched standard compares the bone mineral density of the mammalian subject to the bone mineral density of individuals of comparable age, gender, and size. The young normal standard compares the bone mineral density of the mammalian subject to the bone mineral density of a healthy young adult of the same gender.

Blood and urine markers can be used to aide in the diagnosis of osteoporosis as well as in monitoring the progression of osteoporosis and/or the efficacy of a treatment regimen. Examples of markers in the blood and/or urine for assessing bone health include, but are not limited to blood calcium levels, parathyroid hormone, bone-specific alkaline phosphatase (commercial diagnostic assay, Ostase®), osteocalcin (commercial diagnostic assay, Elecsys®N-MID™), tartrate-resistant acid phosphatase-5b (TRAP), N-telopeptide of type I collagen (NTx), C-telopeptide of type I collagen (CTx), deoxypyridinoline (DPD), pyridinium crosslinks, and vitamin D levels. Additional biomarkers of osteoporosis have been described including inhibin A and inhibin B. See, e.g., US Patent Application 2004/0197828; Biermasz, et al., J. Clin. Endocrinol. Metab. 86:3079-3085, 2001, which are incorporated herein by reference.

Other criteria can be used to establish whether a mammalian subject is at risk for osteoporosis and associated risk for bone fracture. Examples include but are not limited to age, sex, glucocorticoid use, secondary osteoporosis, low body mass index (BMI), the degree of bone turnover, a prior fracture, a family history of fracture, rheumatoid arthritis and lifestyle risk factors such as physical inactivity, smoking, and excessive alcohol consumption. See, e.g., World Health Organization, “WHO Scientific Group on the Assessment of Osteoporosis at Primary Health Care Level” Summary Meeting Report, Brussels, Belgium, 5-7 May 2004, which is incorporated herein by reference.

### Other Bone Loss Diseases and Disorders

A method is described for treating other bone loss diseases or bone loss disorders. Examples of other bone loss diseases or bone loss disorders include, but are not limited to, osteomyelitis, Paget's bone disease, periodontitis, hypercalcemia, osteonecrosis, osteosarcoma, osteolyic metastases, familial expansile osteolysis, prothetic loosening, periprostetic osteolysis, juxtaarticular bone destruction in rheumatoid arthritis, or cleiodocranial dysplasia.

Methods for diagnosis of other bone loss diseases or bone loss disorders include many of the methods described herein for diagnosis and treatment of osteoporosis including x-rays and assessment of bone markers. For example, Paget's bone disease is typically diagnosed using x-ray imaging and analysis of serum alkaline phosphatase levels. Bones affected by Paget's bone disease have a characteristic structural appearance that is apparent in the x-ray images. Levels of serum alkaline phosphatase that are greater than twice the typical levels (20 to 120 units) in an aged match individual may be indicative of Paget's bone disease. In addition to x-ray imaging and blood tests, diagnosis of Paget's bone disease can also include a bone scan. Whereas x-rays, CT scans and MRI examination evaluate the structure of the bone, a bone scan evaluates the functional aspect of bone diseases. In a bone scan, a short-lived radiolabeled tracer, e.g., Technetium^{99m}, is used to detect overactive areas of bone metabolism and turnover. See, e.g., Tang & Chan, Singapore Medical Journal 24:61-72, 1982, which is incorporated herein by reference.

### Follicle-Stimulating Hormone Modulators

The treatment regimen for treating a bone loss disease or a bone loss disorder in a mammalian subject includes providing at least one follicle-stimulating hormone modulator configured to and in an amount sufficient to reduce bioactivity or bioavailability of follicle-stimulating hormone in the mammalian subject. The follicle-stimulating hormone (FSH) modulator can be, e.g., an inhibitor of FSH synthesis and/or secretion, an inhibitor of FSH binding activity, an inhibitor or antagonist of the FSH receptor, or a combination thereof.

The treatment regimen can include at least one FSH modulator that inhibits the synthesis and/or secretion of FSH. FSH is normally synthesized in the anterior pituitary gland in response to the hypothalamic hormone gonadotropin releasing hormone (GnRH). Antagonists of GnRH are able to inhibit the release of FSH in a dose dependent manner. Examples of GnRH antagonists for use in reducing serum levels of FSH include, but are not limited to, the synthetic decapeptides ganirelix (Orgalutran®) and cetrorelix (Cetrotide®). Both are well tolerated with the most common adverse effects being nausea and headache. Other examples of peptide GnRH antagonists include, but are not limited to, degarelix, abarelix (Planaxis™), acyline, and other synthetic decapeptides and nonapeptides. See, e.g., Karten & Rivier Endocrine Rev. 7:44-66, 1986; Beer Rev. Urol. 6(Suppl 7):S33-S38, 2004; Herbst, et al., J. Clin. Endocrinol. Metab. 87:3215-3220, 2002; Samant, et al., J. Med. Chem. 50:2067-2077, 2007; U.S. Pat. No. 6,288,078; U.S. Pat. No. 7,109,171; U.S. Pat. No. 7,285,528; US Patent Application 2007/0015714; U.S. Patent Application 2009/0105153 which are incorporated herein by reference.

In other aspects, the inhibitor of FSH synthesis and/or secretion can be at least one of a small molecule antagonist of GnRH. An example of a small molecule antagonist of GnRH includes, but is not limited to, orally active NBI-56418. See, e.g., Elagolix; Dmowski US Obstetrics & Gynecology, 2008; Struthers, et al., J. Clin. Endocrinol. Metab. 94:545-551, 2009, which are incorporated herein by reference. Other examples of small molecule antagonists of GnRH have been described. See, e.g., U.S. Pat. No. 6,288,078; U.S. Pat. No. 7,495,110; U.S. Pat. No. 7,514,570; US Patent Application 2006/0264631; US Patent Application 2007/0167428; US Patent Application 2008/0108623; US Patent Application 2009/0048273; US Patent Application 2009/0062258, which are incorporated herein by reference.

In other aspects, the at least one inhibitor of FSH synthesis and/or secretion is the polypeptide inhibin. The peptide hormones inhibin A and particularly inhibin B are natural inhibitors of FSH synthesis and secretion. Inhibin A and B are secreted by the ovaries. The levels of inhibin A and B decrease during the transition from perimenopause to postmenopause concomitant with the gradual shut down in ovary function and the increase in circulating FSH. The premenopause levels of inhibin B, for example, are about 55 ng/liter while the postmenopausal levels are about 27 ng/liter. See, e.g., Burger, et al., J. Clin. Endocrinol. Metab. 84:4025-4030, 1999, which is incorporated herein by reference. In some aspects, recombinant inhibin A and/or inhibin B, or analogs, or mimetics thereof, can be administered to a mammalian subject to reduce the level of circulating FSH. See, e.g., Tilbrook, et al., Biol. Reprod. 49:779-788, 1993, which is incorporated herein by reference.

In other aspects, a treatment regimen including the at least one inhibitor of FSH synthesis and/or secretion can be an antagonist of the polypeptide activin. Activin is a naturally occurring activator of FSH biosynthesis and infusion of exogenous activin into a female mammalian subject leads to an increase in circulating FSH. See, e.g., Stouffer, et al., Biol. Reprod. 50:888-895, 1994, which is incorporated herein by reference. Inhibitors of activin activity is configured to decrease the circulating levels of FSH. Inhibitors of activin include, but are not limited to, the activin-binding glycoproteins follistatin and FLRG. See, e.g., U.S. Pat. No. 7,208,470; Razanajaona, et al., Cancer Res. 67:7223-7229, 2007, which is incorporated herein by reference. In some aspects, the activin antagonist can be a soluble activin receptor that selectively binds activin and removes activin from circulation or an antibody that binds the activin receptor and blocks activin binding in the mammalian subject. See, e.g., U.S. Pat. No. 6,982,319; US Patent Application 2009/0087433; US Patent Application 2009/0099086; US Patent Application 2009/0188188.

In other aspects, the at least one inhibitor of FSH synthesis and/or secretion can be the polypeptide follistatin. Follistatin is a natural inhibitor of FSH secretion. Follistatin is secreted from the ovaries and has been shown to bind activin. The actions of follistatin to suppress FSH may be attributable to its capacity to bind and neutralize activin in the pituitary gland. In some aspects, a treatment regimen including recombinant follistatin can be administered to a mammalian subject to reduce the level of circulating FSH. See, e.g., Tilbrook, et al., Biol. Reprod. 53:1353-1358, 1995, which is incorporated herein by reference.

In other aspects, a treatment regimen including the at least one inhibitor of FSH synthesis and/or secretion can be an oligonucleotide that inhibits the synthesis of FSH by gene silencing. In some aspects, gene silencing is performed using single stranded anti-sense RNA. In other aspects, gene silencing is done using RNA interference with short interfering RNA (siRNA), longer double stranded RNA (dsRNA), and/or short hairpin RNA (shRNA). siRNAs are short 19-23 nucleotide duplexes designed to target complementary coding and non-coding regions of a target messenger RNA (mRNA) and including 2 nucleotide, 3-prime overhangs. The dsRNA and shRNA are recognized by the RNase III enzyme Dicer and cut into smaller ˜21 nucleotide siRNAs with 2 nucleotide, 3-prime overhangs. The 5 prime ends are phosphorylated and these small RNAs duplexes are assembled into RNA-induced silencing complexes that ultimately bind to and cleave the target mRNA. At least one siRNA for use in modifying FSH synthesis and secretion can be generated by chemical synthesis, in vitro transcription, siRNA expression vectors, PCR expression cassettes, or a combination thereof. In some aspects, siRNAs for use in targeting FSH mRNA are available from commercial sources or can be custom synthesized (from, e.g., Santa Cruz Biotechnology, Inc., Santa Cruz, Calif.; Applied Biosystems, Inc., Foster City, Calif.). See, e.g., Kim & Rossi, Nat. Rev. Genet. 8:173-184, 2007; Rana Nat. Rev. Mol. Cell Biol. 8:23-36, 2007; Juliano, et al., Nucleic Acids Res. 36:4158-4171, 2008, which are incorporated herein by reference.

In other aspects, the treatment regimen can include at least one FSH modulator that binds and neutralizes FSH. In this instance, the FSH modulator can bind to and remove free FSH from circulation preventing it from binding to the endogenous FSH receptor. Examples of FSH modulators that can be used to neutralize free FSH include, but are not limited to, endogenous FSH binding proteins, FSH specific antibodies, all or part of the FSH receptor, or a combination thereof.

In other aspects, the treatment regiment can include at least one FSH modulator that is an antibody that binds free FSH in circulation and prevents it from interacting with the FSH receptor. Antibodies or fragments thereof for use in neutralizing FSH can include, but are not limited to, monoclonal antibodies, polyclonal antibodies, human antibodies, humanized antibodies or antibody fragments, Fab fragments of antibodies, Fab₂ fragments of antibodies, single-chain variable fragments (scFvs) of antibodies, diabody fragments (dimers of scFvs fragments), minibody fragments (dimers of scFvs-C_{H}3 with linker amino acid), or the like. Antibodies or fragments thereof for use in neutralizing FSH can be generated against FSH using standard methods such as those described by Harlow & Lane, Antibodies: A Laboratory Manual, Cold Spring Harbor Laboratory Press; 1^{st} edition 1988, which is incorporated herein by reference. Alternatively, an antibody fragment directed against FSH can be generated using phage display technology. See, e.g., Kupper, et al. BMC Biotechnology 5:4, 2005, which is incorporated herein by reference. An antibody or fragment thereof could also be prepared using in silico design. See, Knappik et al., J. Mol. Biol. 296: 57-86, 2000, which is incorporated herein by reference.

In other aspects, the treatment regimen can include at least one FSH modulator that is all or part of the FSH receptor capable of binding free FSH in circulation and preventing it from interacting with the endogenous FSH receptor. The FSH receptor is a membrane associated G-protein coupled receptor (GPCR). The FSH receptor can be incorporated into liposomes or other membrane vesicles and used to neutralize circulating FSH. In other aspects, a soluble portion of the FSH receptor is used to bind and neutralize circulating FSH. For example, the soluble form of the FSH receptor can be a soluble receptor fragment that includes the ectodomain of the FSH receptor responsible for binding FSH. The soluble receptor fragment can be synthesized and administered alone or as part of a larger fusion protein. See, e.g., Osuga, et al., Mol. Endocrinol. 11:1659-1668, 1997, which is incorporated herein by reference.

The treatment regimen can include at least one modulator of FSH that antagonizes or inhibits the activity of the FSH receptor. The modulator of the FSH receptor can be a naturally-occurring antagonistic or a mimetic thereof. Examples of naturally occurring antagonists of the FSH receptor include, but are not limited to, a wide spectrum of FSH isohormone forms that exhibit FSH antagonist activity by binding to the FSH receptor without eliciting a response; specific anti-FSH antibodies present in the circulation; and various proteins that inhibit FSH action, either by interfering with binding of FSH to the receptor or at the level of signal transduction. See, e.g., Fauser Mol. Hum. Reprod. 2:327-334, 1996, which is incorporated herein by reference. In some aspects, the modulator of the FSH receptor can include, but is not limited to, a modified FSH polypeptide or other polypeptide, a small molecule antagonist, an antibody, a steroid, an oligonucleotide, or a combination thereof.

The treatment regimen can include at least one modulator of the FSH receptor that is a modified form of FSH. FSH is a heterodimeric glycoprotein with two N-linked glycosylation sites on each subunit that are essential for binding and activation of the FSH receptor. FSH polypeptides with modified glycosylation states or chemically deglycosylated exhibit altered interaction with the FSH receptor. For example, FSH that has been purified and chemically deglycosylated with hydrogen fluoride can inhibit the activity of FSH receptor as measured by decreased accumulation of cAMP. Similarly, recombinant FSH expressed in Hi5 insect cells has a modified glycosylation pattern and inhibits the activity of the FSH receptor. See, e.g., Avey, et al., Mol. Endocrinol. 11:517-526, 1997, which is incorporated herein by reference.

In some aspects, the inhibitor or antagonist of the FSH receptor can be a small molecule. A variety of small molecule inhibitors of FSH receptors have been described including but not limited to tetrahydroquinolines (US Patent Applications US 2004/0236109; US 2006/0167047; van Straten, et al., J. Med. Chem. 48:1697-1700, 2005), diketopiperazines (U.S. Pat. No. 6,900,213), sulphonamides (U.S. Pat. No. 6,583,179), thiazolidinones (U.S. Pat. No. 6,426,357), sulphonic acids (U.S. Pat. No. 6,200,963; U.S. Pat. No. 6,355,633), azo compounds (US Patent Applications 2009/0082372, US 2008/0275083, Arey, et al., Endocrinology 143:3822-3829, 2002), pyrrolobenzodiazepines (US Patent Applications US 2006/0199806, US 2006/0258644, US 2006/0258645, US 2006/0287522), acyltryptophanols (US Patent Applications US 2008/0221195, US 2009/0075987), which are incorporated herein by reference.

In some aspects, the inhibitor or antagonist of the FSH receptor can be an antibody. The antibody can bind to the FSH binding domain of the receptor and block endogenous ligand binding. Antibodies or fragments thereof for use in blocking the activity of the FSH receptor can include, but are not limited to, monoclonal antibodies, polyclonal antibodies, human antibodies, humanized antibodies or antibody fragments, Fab fragments of antibodies, Fab₂ fragments of antibodies, single-chain variable fragments (scFvs) of antibodies, diabody fragments (dimers of scFvs fragments), minibody fragments (dimers of scFvs-C_{H}3 with linker amino acid), or the like. Antibodies or fragments thereof for use blocking the activity of the FSH receptor can be generated against the FSH receptor using standard methods such as those described by Harlow & Lane, Antibodies: A Laboratory Manual, Cold Spring Harbor Laboratory Press; 1^{st} edition 1988, which is incorporated herein by reference. Alternatively, an antibody fragment directed against the FSH receptor can be generated using phage display technology. See, e.g., Kupper, et al. BMC Biotechnology 5:4, 2005, which is incorporated herein by reference. An antibody or fragment thereof could also be prepared using in silico design (Knappik et al., J. Mol. Biol. 296: 57-86, 2000, which is incorporated herein by reference. In some aspects, antibodies that block the activity of the FSH receptor can be generated in vivo within the treated subject. For example, the subject can be immunized with all or part of the FSH receptor and can mount an immune response that results in generation of antibodies that block the activity of the FSH receptor. See, e.g., Moudgal, et al., Endocrinol. 138:3065-3068, 1997, which is incorporated herein by reference.

### Treatment Regimen for Osteoporosis and Other Bone Loss Diseases and Disorders

The treatment regimen for treating a bone loss disease or disorder can include providing at least one follicle stimulating hormone (FSH) modulator optionally in combination with one or more steroid hormones or metabolites or modulators thereof, and optionally in combination with other medications for treating osteoporosis or other bone loss diseases or disorders. Other medications used to treat osteoporosis include, but are not limited to, hormone replacement therapy (e.g., estrogen with or without progestin), bisphosphonates (e.g., etidronate, pamidronate, alendronate, risedronate, tiludronate, ibandronate, and zoledronic acid), selective estrogen receptor modulators (SERMs; e.g., raloxifene (Evista®), tamoxifen), calcitonin (Miacalcin®, Fortical®), teriparatide (recombinant form of parathyroid hormone 1-34, Forteo®), vitamin D (e.g., calcitriol, cholecalciferol, doxercalcirerol, ergocalciferol, paricalcitol) and calcium (e.g., calcium acetate, calcium carbonate, calcium chloride, calcium citrate, calcium glubionate, calcium gluceptate, calcium gluconate, calcium lactate, tricalcium phosphate).

### Treatment Regimen Including Replacement Therapy for One or More Steroid Hormones

The treatment regimen for treating a bone loss disease or disorder can include providing a follicle stimulating hormone (FSH) modulator, optionally in combination with replacement therapy that includes one or more steroid hormones or metabolites or modulators thereof. The at least one treatment regimen including replacement therapy includes a pharmaceutical composition of one or more of the compounds or compositions as described herein, including but not limited to, natural or synthetic compounds with estrogenic activity; synthetic steroidal compounds having estrogenic activity; synthetic non-steroidal compounds having estrogenic activity; plant-derived phytoestrogens having estrogenic activity; esters, conjugates or prodrugs of suitable estrogens; androgens; modulators, including but not limited to selective estrogen receptor modulators (SERMs) and modulators of metabolic and/or synthetic pathways such as enzyme regulators; and modulators of signaling pathways, progesterones; natural or synthetic compounds having progestational activity; or analogs, metabolites, hormone precursors, metabolite precursors, biosynthetic enzymes, DNA encoding biosynthetic enzymes, or derivatives thereof. The compound or composition further includes analogs, peptide mimetics, DNA encoding polypeptides of interest, or small chemical molecular mimetics of the one or more steroid hormones, or metabolites or modulators.

Compounds that can be used as part of the treatment regimen include at least one selective estrogen receptor modulator (SERM). Examples of SERMs can include, but are not limited to, tamoxifen, idoxifene, toremifene and raloxifene. The selective estrogen receptor modulators can include, but are not limited to, at least one selective estrogen receptor a agonist and/or at least one selective estrogen receptor β agonist. The at least one selective estrogen receptor a agonist can include, but is not limited to, 17β-estradiol or propylpyrazole triol, 3,17-dihydroxy-19-nor-17α-pregna-1,3,5 (10)triene-21,16α-lactone. See, e.g., Proc. Natl. Acad. Sci. USA 101: 5129-5134, 2004, which is incorporated herein by reference. The at least one selective estrogen receptor β agonist can include, but is not limited to, diarylpropionitrile, ERB-041 [Harris et al., Endocrinology 144: 4241-4249, 2003], WAY-202196, WAY-214156 (2,8-dihydroxy-6H-dibenzo[c,h]chromene-4,12-dicarbonitrile), 8-vinylestra-1,3,5 (10)-triene-3,17β-diol, or a selective estrogen receptor modulator. See, e.g., Cvoro et al., J. Immunol., 180: 630-636, 2008; Proc. Natl. Acad. Sci. USA 101: 5129-5134, 2004, which is incorporated herein by reference.

Pharmaceutical compounds and compositions that can be used to alter estrogen levels, for example, can include, but are not limited to, natural compounds with estrogenic activity such as estradiol (estradiol-17β), estriol, estrone, and their metabolites such as 2-hydroxyestrone, 2-methoxyestrone, 16α-hydroxyestrone, 17α-estradiol, 2-hydroxy-estradiol-17β, 2-methoxyl-estradiol-17β 6β-hydroxyl-estradiol-17β, 3-sulfate, 3-glucuronide, and 16-glucuronide; synthetic steroidal compounds having estrogenic activity such as estradiol 17β-acetate, estradiol 17β-cypionate, estradiol 17β-propionate, estradiol 3-benzoate, ethinyl estradiol, piperazine estrone sulfate, mestranol, and quinestrol; synthetic non-steroidal compounds having estrogenic activity such as diethylstilbestrol, chlorotrianisene, and methallenestril; and plant derived phytoestrogens having estrogenic activity such as coumestrol, 4′methoxycoumestrol, repensol, trifoliol, daidzein, formononetin, genistein, and biochanin A. Esters, conjugates and prodrugs of suitable estrogens can also be used. Examples of estrogen prodrugs that can be used include, but are not limited to, estradiol acetate (which is converted in vivo to 17β-estradiol) and mestranol (which is converted in vivo to ethinyl estradiol). In some instances, a combination of estrogens can be used, e.g., to provide a combination of three estrogens 2-hydroxyestrone, 17-β estradiol, and estriol, for example in a ratio determined by the method. Further examples of 17β-estradiol compositions for use in the treatment regimen include oral tablets (e.g., Estrace®, Progynova®), transdermal patches (e.g., Estraderm®, Alora®, Climara®, Menostar™), topical creams (e.g., Estrasorb™ EstroGel®, Elestrin™), and a vaginal ring (e.g., Estring®). See, e.g. U.S. Pat. No. 6,911,438, which is incorporated herein by reference.

In some aspects, the pharmaceutical compounds and compositions used to alter a hormone level, can include a natural precursor. For example, steroid hormone levels can be altered by providing a natural precursor, for example, testosterone, that can be converted in vivo to estradiol, or androstenedione, that, in turn, can be converted to estrone or can be converted to testosterone. The treatment regimen can include a compound with enzymatic activity configured to convert a naturally occurring precursor so as to alter a hormone level, for example a cytochrome P450 enzyme, or analog or modulator thereof. The treatment regimen can include modulating the activity of a resident enzyme, such as one active in steroidogenesis, by adding an inhibitor or activator.

Pharmaceutical compounds and compositions that can be used as part of a treatment regimen to alter progesterone levels, for example, can include, but are not limited to, natural and synthetic compounds having progestational activity, for example, progesterone, levonorgestrel, norethindrone, norethindrone acetate, desogestrel, gestodene, dienogest, norgestimate, cyproterone acetate, norelgestromin, etonogestrel, ethynodiol diacetate, norgestrel, trimegestone, medroxyprogesterone acetate, chlormadinone acetate, drospirenone, and other natural and/or synthetic gestagens. Esters, conjugates, and prodrugs of suitable progestins can also be used. Additional compounds can include metabolites and/or analogs of progesterone, for example, 20α-DH-P (4-pregnen-20α-ol-3-one), 5α-DH-P (5α-pregnan-3,20-dione), 3β,5α-TH-P (5α-pregnan-3b-ol-20-one), 20α-DH,5α-DH-P (5α-pregnan-20α-ol-3-one), 16α-OH—P (4-pregnen-16α-ol-3,20-dione), 5β-DH-P (5β-pregnan-3,20-dione), 20α-DH,3β,5α-TH-P (5α-pregnan-3β,20α-diol), 20α-DH, 3α,5α-TH-P (5α-pregnan-3α,20α-diol), 3α,5α-TH-P (5α-pregnan-3α-ol-20-one), 11α-OH—P (4-pregnen-11α-ol-3,20-dione), 11β-OH—P (4-pregnen-17α-ol-3,20-dione), 20α-DH,3α,5β-TH-P (5β-pregnan-3α,20α-diol), 17α-OH—P (4-pregnen-17α-ol-3-one), 17α-OH,20α-DH-P (4-pregnen-17,20α-diol-3-one) and 3α,5β-TH-P (5β-pregnan-3α-ol-20-one). See, e.g., Quinkler, et al., Eur. J. Endocrinol. 146:789-800, 2002, which is incorporated herein by reference. Further examples of progesterone compositions for use in the treatment regimen include Provera®, Megace®, and Aygestin®.

### Assays for Measuring Follicle Stimulating Hormone in a Mammalian Subject

A treatment regimen including at least one FSH modulator for treating a bone loss disease or bone loss disorder is based on measurements of the cyclic physiological pre-disease levels of FSH, or steroid hormone levels, in the mammalian subject and on current cyclic levels of FSH, or steroid hormone levels, in the mammalian subject. A physiological pre-disease level can be a level of FSH as measured in a female population or a male population at a point in time prior to occurrence of disease in the population. Alternatively, a physiological pre-disease level can be a level of FSH as measured at a point in time prior to occurrence of disease or prior to surgery to treat a disease in the female subject or male subject. In some aspects, the physiological pre-disease levels of FSH in a female subject can be the same as physiological premenopausal levels of FSH in the female subject. A time-history profile of FSH levels for the female subject can be generate using periodic measurements of cyclic physiological pre-disease levels of FSH as well as current FSH levels of the female subject. In some aspects, the pre-disease levels of FSH in the mammalian subject are measured periodically as part of a routine medical checkup. FSH levels can be measured over any variety of time intervals including but not limited to one or more days, one or more weeks, one or more months, one or more years. In a female subject who is premenopausal or perimenopausal and pre-disease, the levels of FSH can be measured at various time points over the course of one or more menstrual cycles to provide a pre-disease profile of the cyclic physiological FSH levels. The current FSH levels of a mammalian subject can be measured concurrent with the diagnosis of a bone disease and/or at a later date prior to initiation of the treatment regimen.

In some aspects, a physiological pre-disease level can be a level of FSH or steroid hormone levels as measured in a general population of male subjects or female subjects, e.g., in a healthy population, at a point in time prior to occurrence of disease or prior to surgery to treat a disease in the female subject or the male subject. In a female population who is premenopausal or perimenopausal and pre-disease, the levels of FSH can be measured at various time points over the course of one or more menstrual cycles to provide a pre-disease profile of the cyclic physiological FSH levels. The FSH levels of a mammalian subject population with a bone disease or disorder can be measured concurrent with the diagnosis of a bone disease and/or at a later date prior to initiation of the treatment regimen in the mammalian population.

The information regarding the pre-disease and current FSH levels of a mammalian subject can be stored, analyzed and tracked in the mammalian subject's medical record. Methods for storing this information include paper storage as well as electronic storage. Analysis and tracking can be done manually by looking at the data. Ideally, a software program is designed and used to store, analyze and track the time-history profile of FSH levels of a mammalian subject. The software program can be used to monitor changes in the time-history profile of FSH levels of a mammalian subject from one measurement period to the next. The software program can compare the time history of FSH levels of a mammalian subject relative to FSH levels associated with an age-matched population norm. The software program can also compare the FSH levels of a mammalian subject to a cyclic physiological level of FSH. The cyclic physiological level of FSH can be inferred by measuring FSH levels at a time in the mammalian subject's life when FSH levels are assumed to be within a “normal range”. For example, in the case of a female subject, this can be during premenopause. The time-history of FSH levels can be used to monitor changes in levels of FSH of either a female subject's physiological level of FSH or that of a population norm. The time-history profile of FSH levels of a female subject is used to develop a treatment regimen with an FSH modulator, or optionally with a steroid hormone modulator, that allows the female subject's levels of FSH to approach a target cyclic physiological pre-disease level of FSH.

One or more assay systems can be used to measure the levels of FSH in the blood, urine or other body fluid or tissue of a mammalian subject. The one or more assay systems can incorporate any of a number of binding type assays that use a specific FSH binding moiety to capture and measure the relative amount of FSH present in a bodily fluid. The FSH binding moiety can be the FSH receptor itself, an FSH-specific antibody, an FSH-specific aptamer, an artificial FSH binding substrate, and/or other FSH binding moieties.

In some aspects, the assay system for measuring the levels of FSH is an immunoassay that uses one or more FSH-specific antibodies to measure the concentration of FSH in a mammalian subject's blood, urine or other body fluid. Examples of immunoassays include but are not limited to radioimmunoassays (RIA), immunometric assays (IMA), enzyme-linked immunosorbent assays (ELISA), and chemiluminescence immunoassays (CLIA). In some aspects, the immunoassay is a competitive immunoassay in which FSH in the blood, urine or other body fluid of a mammalian subject competes with labeled FSH for binding to the one or more FSH-specific antibodies. In this instance, the measured response is inversely proportional to the concentration of the FSH in the biological sample. In other aspects, the immunoassay is a noncompetitive assay or sandwich assay in which FSH in the blood, urine or other body fluid of a mammalian subject is bound to one FSH-specific antibody. A second FSH-specific antibody that includes a detectable label is bound to the FSH and provides a measurable readout. In this instance, the measured response is proportional to the concentration of FSH in the biological sample. The FSH and/or one or more FSH-specific antibodies for use in the immunoassay can be labeled for detection. Examples of labels for detection include, but not limited to, an enzyme linked to a color reaction, bioluminescent and/or chemiluminescent chemical reaction, colloidal gold, radioisotopes, magnetic labels, fluorescent fluorophore, or a combination thereof. Additional examples of labels for detection include, but are not limited to, lanthanide chelates (e.g., europium(III), terbium(III), samarium(III), and dysprosium(III)), quantum dots, luminescent inorganic crystals, up-converting phosphors, fluorescent nanoparticles, and plasmon resonant particles. See, e.g., Soukka, et al., Clin. Chem. 47:1269-1278, 2001, which is incorporated herein by reference.

The one or more FSH-specific antibodies for use in an immunoassay can include, but are not limited to, monoclonal antibodies, polyclonal antibodies, human antibodies, humanized antibodies or antibody fragments, Fab fragments of antibodies, Fab₂ fragments of antibodies, single-chain variable fragments (scFvs) of antibodies, diabody fragments (dimers of scFvs fragments), minibody fragments (dimers of scFvs-C_{H}3 with linker amino acid), or the like. Antibodies or fragments thereof for use in an FSH diagnostic immunoassay can be generated against FSH using standard methods such as those described by Harlow & Lane, Antibodies: A Laboratory Manual, Cold Spring Harbor Laboratory Press; 1^{st} edition 1988, which is incorporated herein by reference. Alternatively, an antibody fragment directed against FSH can be generated using phage display technology. See, e.g., Kupper, et al. BMC Biotechnology 5:4, 2005, which is incorporated herein by reference. An antibody or fragment thereof could also be prepared using in silico design. See, e.g., Knappik et al., J. Mol. Biol. 296: 57-86, 2000, which is incorporated herein by reference.

A number of commercially available immunoassay diagnostic kits are available for measuring FSH in blood, urine or other body fluids. Examples include various in-line dip-stick urine testing devices for home use (from, e.g., IND Diagnostics, Inc. Foster City, Calif.; The Lifestyle Company, Inc., Wall, N.J.; ACON Laboratories, Inc. San Diego, Calif.). In general, these assay systems use an immobilized antibody against FSH on a chromatography matrix, e.g., nitrocellulose membrane. A second antibody against FSH that is tagged with a colorimetric dye is supported in a separate portion of the matrix. As urine is wicked through the matrix, it binds the second FSH antibody and the complex is captured by the immobilized FSH antibody, creating a band of color. The intensity of the band of color is compared with a band of color generated by a standard. These types of assays provide a qualitative measure of FSH in the urine of a mammalian subject. Other commercial immunoassay systems for use in assaying FSH include fully automated diagnostics systems for clinical laboratory use (e.g., Cobas®6000, Roche Diagnostics Corp, Indianapolis, Ind.; UniCel® DxC 600i Synchron® Access® Clinical System, Beckman Coulter, Fullerton, Calif.).

In other aspects, the levels of FSH in the blood, urine or other body fluid or tissue of a mammalian subject can be measured using a surface plasmon resonance immunosensor. See, e.g., Trevino, et al., Clin. Chim. Acta 403:56-62, 2009, which is incorporated herein by reference. In some aspects, FSH is immobilized on the sensor surface. FSH in the sample of blood, urine or other body fluid competes with the immobilized FSH for binding to a FSH-specific binding moiety. The binding moiety can be an antibody, an aptamer, all or part of the FSH receptor, or other composition that selectively binds FSH. The resulting surface plasmon resonance output signal is proportional to the amount of FSH-specific binding moiety that binds to the sensor and inversely proportional to the amount of FSH in the biological sample.

In some aspects, the levels of FSH in the blood, urine or other body fluid or tissue of a mammalian subject is measured by competitive binding to the FSH receptor in which FSH in the biological sample competes with labeled FSH for binding to the FSH receptor. The amount of FSH in the sample is inversely proportional to the measured response. Labeled FSH for use in the receptor binding assay can be labeled with an enzyme linked to a color reaction, bioluminescent and/or chemiluminescent chemical reaction, colloidal gold, radioisotopes, magnetic labels, fluorescent fluorophore, lanthanide chelates (e.g., europium(III), terbium(III), samarium(III), and dysprosium(III)), quantum dots, luminescent inorganic crystals, up-converting phosphors, fluorescent nanoparticles, plasmon resonant particles, or combinations thereof. The FSH receptor for use in the binding assay can be naturally associated with a mammalian cell. Examples of cells that naturally express the FSH receptor include, but are not limited to, granulosa cells, Sertoli cells, and osteoclasts. Alternatively, the FSH receptor can be genetically engineered into a cell line using standard molecular biology techniques. See, e.g., Gudermann, et al., Endocrinol. 135:2204-2213, 1994, which is incorporated herein by reference. In other aspects, all or part of the FSH receptor is isolated from a natural source or genetically engineered cell line and either maintained in cell membranes or placed into an artificial membrane. For example, studies describe a radioligand receptor assay for FSH in which serum-derived FSH and iodinated FSH compete for binding to a homogenized membrane preparation from bovine testes that includes intact FSH receptors. See, e.g., Schneyer, et al., Clin. Chem. 37:508-514, 1991, which is incorporated herein by reference. Alternatively, all or part of the FSH receptor can be isolated, purified and attached to a substrate, e.g., beads, matrix, or microtiter plates, for use in the competitive binding assay.

In other aspects, the levels of FSH in the blood, urine or other body fluid or tissue of a mammalian subject can be measured using a bioassay with a biological readout. The binding of FSH to the FSH receptor normally leads to an increase in the second messenger cAMP. Measuring the production of cAMP can be used to indirectly measure the amount of FSH present in a biological sample. Exemplary cells for use in the FSH bioassay include but are not limited to granulosa cells, Sertoli cells, osteoclast cells, and cells genetically modified with the FSH receptor. The production of cAMP can be measured using a chemiluminescence immunoassay (CLIA) or radioimmunoassay (RIA) using cAMP-specific antibodies, assay kits for which are commercially available (from, e.g., GE Healthcare, Waukesha, Wis.). Other bioassays for assessing FSH include measurements of aromatase activity and estradiol secretion.

### Assays for Measuring Steroid Hormone Levels in a Mammalian Subject

A treatment regimen that includes providing at least one modulator of FSH for treating a bone loss disease or bone loss disorder in a mammalian subject can further include providing replacement therapy with one or more steroid hormones or metabolites or modulators thereof. The treatment regimen including one or more steroid hormones or metabolites or modulators thereof is configured to approach a target cyclic physiological pre-disease level of follicle stimulating hormone and the one or more steroid hormones in the mammalian subject. A physiological pre-disease level can be a level of follicle stimulating hormone and steroid hormone as measured at a point in time prior to occurrence of disease or prior to surgery to treat a disease in a female or male subject. In some aspects, the physiological pre-disease levels of the steroid hormone in a female subject can be the same as the physiological premenopausal levels of steroid hormone in the mammalian subject. Periodic measurements of cyclic physiological pre-disease levels of steroid hormone as well as current steroid hormone levels of a mammalian subject can be used to generate a time-history profile of steroid hormone levels for the mammalian subject. In some aspects, the pre-disease levels of steroid hormone in the mammalian subject are measured periodically as part of a routine medical checkup. Steroid hormones levels can be measured over any variety of time intervals including but not limited to one or more days, one or more weeks, one or more months, one or more years. In a female mammalian subject who is premenopausal or perimenopausal and pre-disease, the levels of one or more steroid hormones can be measured at various time points over the course of one or more menstrual cycles to provide a pre-disease profile of the cyclic physiological steroid hormone levels. The current steroid hormone levels of a mammalian subject can be measured concurrent with the diagnosis of a bone disease and/or at a later date prior to initiation of the treatment regimen.

In some aspects, a physiological pre-disease level can be a level of FSH or one or more steroid hormones levels or metabolites or modulators thereof as measured in a general population of male subjects or female subjects, e.g., in a healthy population, at a point in time prior to occurrence of disease or prior to surgery to treat a disease in the female subject or the male subject. In a premenopausal female population or perimenopausal and pre-disease female population, the levels of FSH or one or more steroid hormones or metabolites or modulators thereof can be measured at various time points over the course of one or more menstrual cycles to provide a pre-disease profile of the cyclic physiological FSH levels. The FSH levels or one or more steroid hormones levels or metabolites or modulators of a mammalian subject population with a bone disease or disorder can be measured concurrent with the diagnosis of a bone disease and/or at a later date prior to initiation of the treatment regimen in the mammalian population.

The information regarding the pre-disease and current steroid hormone levels of a mammalian subject can be stored, analyzed and tracked in the mammalian subject's medical record. Methods for storing this information include paper storage as well as electronic storage. Analysis and tracking can be done manually by looking at the data. Ideally, a software program is designed and used to store, analyze and track the time-history profile of the steroid hormone levels of a mammalian subject. The software program can be used to monitor changes in the time-history profile of the steroid hormone levels of a mammalian subject from one measurement period to the next. The software program can compare the time history of steroid hormone levels of a mammalian subject relative to steroid hormone levels associated with an age-matched population norm. The software program can also compare the steroid hormone levels of a mammalian subject to a cyclic physiological level of steroid hormone. The cyclic physiological level of one or more steroid hormones can be inferred by measuring one or more steroid hormone levels at a time in the mammalian subject's life when the one or more steroid hormone levels are assumed to be within a “normal range”. For example, in the case of a female subject, this can be during premenopause. The time-history of one or more steroid hormone levels can be used to monitor changes in the levels of one or more steroid hormone relative to either a female subject's physiological level of one or more steroid hormones or that of a population norm. The time-history profile of one or more steroid hormone levels of a female subject is used to develop a replacement therapy for inclusion in the treatment regimen that allows the female subject's levels of one or more steroid hormones to approach a target cyclic physiological pre-disease level of one or more steroid hormones.

One or more assay systems can be used to measure the levels of one or more steroid hormones in the blood, urine or other body fluid or tissue of a mammalian subject. In some aspects, the assay system for measuring the levels of one or more steroid hormones is an immunoassay that uses one or more steroid hormone-specific antibodies to measure the concentration of steroid hormone in a mammalian subject's blood, urine or other body fluid. Examples of immunoassays include but are not limited to radioimmunoassays (RIA), immunometric assays (IMA), enzyme-linked immunosorbent assays (ELISA), and chemiluminescence immunoassays (CLIA). In some aspects, the immunoassay is a competitive immunoassay in which the steroid hormone in the blood, urine or other body fluid of a mammalian subject competes with labeled steroid hormone for binding to the one or more steroid hormone-specific antibodies. In this instance, the measured response is inversely proportional to the concentration of the steroid hormone in the biological sample. In other aspects, the immunoassay is a noncompetitive assay or sandwich assay in which the steroid hormone in the blood, urine or other body fluid of a mammalian subject is bound to one steroid hormone-specific antibody. A second steroid hormone-specific antibody that includes a detectable label is bound to the steroid hormone and provides a measurable readout. In this instance, the measured response is proportional to the concentration of steroid hormone in the biological sample. The steroid hormone and/or one or more steroid hormone-specific antibodies for use in the immunoassay can be labeled for detection. Examples of labels for detection include, but not limited to, an enzyme linked to a color reaction, bioluminescent and/or chemiluminescent chemical reaction, colloidal gold, radioisotopes, magnetic labels, fluorescent fluorophore, or a combination thereof. Additional examples of labels for detection include, but are not limited to, lanthanide chelates (e.g., europium(III), terbium(III), samarium(III), and dysprosium(III)), quantum dots, luminescent inorganic crystals, up-converting phosphors, fluorescent nanoparticles, and plasmon resonant particles. See, e.g., Soukka, et al., Clin. Chem. 47:1269-1278, 2001, which is incorporated herein by reference.

Antibodies or fragments thereof for use in an immunoassay can be generated against a steroid hormone using standard methods, for example, such as those described by Harlow & Lane, Antibodies: A Laboratory Manual, Cold Spring Harbor Laboratory Press; 1^{st} edition 1988, which is incorporated herein by reference. Alternatively, an antibody fragment directed against a steroid hormone can be generated using phage display technology. See, e.g., Kupper, et al. BMC Biotechnology 5:4, 2005, which is incorporated herein by reference. An antibody or fragment thereof could also be prepared using in silico design. See, e.g., Knappik et al., J. Mol. Biol. 296: 57-86, 2000, which is incorporated herein by reference. In addition or instead of an antibody, the assay can employ another type of recognition element, such as a receptor or ligand binding molecule. Such a recognition element can be a synthetic element like an artificial antibody or other mimetic. See, e.g., U.S. Pat. No. 6,255,461 (Artificial antibodies to corticosteroids prepared by molecular imprinting), U.S. Pat. No. 5,804,563 (Synthetic receptors, libraries and uses thereof), U.S. Pat. No. 6,797,522 (Synthetic receptors), U.S. Pat. No. 6,670,427 (Template-textured materials, methods for the production and use thereof), and U.S. Pat. No. 5,831,012, U.S. Patent Application 20040018508 (Surrogate antibodies and methods of preparation and use thereof); and Ye and Haupt, Anal Bioanal Chem. 378: 1887-1897, 2004; Peppas and Huang, Pharm Res. 19: 578-587 2002, which provide examples of such synthetic elements and are incorporated herein by reference. In some instances, antibodies, recognition elements, or synthetic molecules that recognize a hormone can be available from a commercial source, e.g., Affibody® affinity ligands (Abcam, Inc. Cambridge, Mass. 02139-1517; U.S. Pat. No. 5,831,012, incorporated herein by reference. For example, antibodies to estradiol, estrone, estriol, testosterone, DHEA, progesterone, follicle stimulating hormone, luteinizing hormone and estrogen receptors α and β are available from numerous commercial sources as listed in the Linscott's Directory of Immunological & Biological Reagents, Linscott's USA, Mill Valley, Calif. 94941. Similarly, ELISA kits designed to measure one or more hormones are commercially available. For example, ELISA kits for measuring estradiol, estrone, estriol, testosterone, DHEA, progesterone, follicle stimulating hormone, luteinizing hormone (from, e.g., Cayman Chemical, Ann Arbor, Mich.; Calbiotech, Spring Valley, Calif.; Beckman Coulter, Fullerton, Calif.). Other biomolecules can be developed to selectively bind to steroid hormones or related molecules, modulators or metabolites, for example, DNA or RNA oligonucleotide based aptamers, and used in diagnostic assays. See, e.g., Jayasena. Clin. Chem. 45:1628-1650, 1999, which is incorporated herein by reference.

Alternatively, the levels of one or more steroid hormones in a bodily fluid or tissue of a mammalian subject can be assayed using gas or liquid chromatography with or without mass spectrometry. For example, estradiol and estrone levels in human plasma can be simultaneously measured using a liquid chromatography-tandem mass spectrometry assay. See, e.g. Nelson, et al., Clin. Chem. 50:373-384, 2004, which is incorporated herein by reference. In this instance, the serum samples are derivatized with dansyl chloride to increase the sensitivity of the assay and efficiency of ionization and separated from other components of the serum by liquid chromatography. Further purification and detection is done using mass spectrometry to differentiate between various steroid hormones. A more rapid method for detecting steroid hormones such as estradiol, estrone, estriol, 16-hydroxyestrone, and aldosterone, for example, using liquid chromatography, electrospray ionization and mass spectrometry (LC-ESI-MS/MS) has been described. See, e.g., Guo, et al., Clin. Biochem. 41:736-741, 2008, which is incorporated herein by reference. In this instance, the serum samples are deproteinized by extraction with acetonitrile followed by centrifugation at 13,000 rpm for 10 minutes. The supernatant is then loaded directly into the LC-ESI-MS/MS system where the samples are chromatographed. Standards are used to determine the elution profile of each steroid hormone and the respective peaks are submitted to electrospray ionization followed by mass spectrometry. Known quantities of a given hormone are subjected to the same process and used to generate a standard curve against which the measured levels of hormone in the serum sample are compared.

Levels of one or more steroid hormones can also be assayed in a bodily fluid or tissue using a recombinant cell based assay or biosensor. In one instance, a yeast strain or a mammalian cell line is modified to express a recombinant hormone receptor that emits a measurable readout in response to binding an analyte, such as a steroid hormone. Studies describe development of a bioassay in Saccharomyces cerevisiae that have been transformed with the human estrogen receptor and an estrogen response element (ERE) upstream of the yeast iso-1-cytochrome C promoter fused to the structural gene for β-galactosidase. See, Klein, et al., J. Clin. Endocrinol. Metab. 80:2658-2660, 1995, which is incorporated herein by reference. Increased β-galactosidase activity in response to the presence of estrogen is assessed using colorimetric detection. Alternatively, a luminescent assay system or biosensor can be used to measure estrogen levels by incorporating human estrogen receptor α and/or β into a mammalian cell line in combination with an estrogen-responsive element (ERE) upstream of a luciferase gene reporter. See, Paris, et al., J. Clin. Endocrinol. Metab. 87: 791-797, 2002, which is incorporated herein by reference.

Levels of one or more steroid hormones can be measured using sensor technology, including for example, chemical sensors, biosensors, protein arrays, and/or microfluidic devices, that can also be referred to as “lab-on-a-chip” systems. See, e.g., Cheng, et al., Anal. Chem. 73: 1472-1479, 2001; Bange, et al., Biosensors Bioelectronics 20: 2488-2503, 2005; De, et al., J. Steroid Biochem. Mol. Biol. 96: 235-244, 2005; Zhou, et al., Sci. China C. Life Sci. 49: 286-292, 2006; Hansen, et al., Nano Lett., 7: 2831-2834, 2007, which are incorporated herein by reference; Dauksaite et al., Nanotech 18(125503): 1-5, 2007). For example, a biosensor can be generated based on the interaction between estradiol and the estrogen receptor. See, e.g., Murata, et al., Anal. Sci. 17:387-390, 2001, which is incorporated herein by reference. In this instance, recombinant estrogen receptor is linked to an Au-electrode and cyclic voltametric measurements are used to assess changes in the properties of the estrogen receptor protein layer in response to estradiol binding.

In some instances, one or more steroid hormones are extracted from the bodily fluid or tissue sample, e.g., blood, serum, plasma, urine, urogenital secretions, sweat and/or saliva, using organic solvents prior to performing one or more of the measurements described above. For example, a hormone, estradiol, can be extracted from serum using a combination of hexane and ethyl acetate followed by mixing, centrifugation, and collection of the organic layer. See, e.g., Dighe & Sluss, Clin. Chem. 50:764-6, 2004, which is incorporated herein by reference. Extracted hormones in the organic layer can be further fractionated using chromatography. For example, testosterone, dihydroestosterone, androstenedione, estrone, and estradiol extracted from serum into an organic layer can be further fractioned using Celite column partition chromatography and eluting solvents such as toluene, isooctane and ethyl acetate. See, e.g., Hsing, et al., Cancer Epidemiol. Biomarkers Prev. 16:1004-1008, 2007, which is incorporated herein by reference. Radiolabeled internal standards corresponding to a given hormone can be used to assess procedural losses.

In some instances, steroid hormone levels in a mammalian subject can be measured transdermally using a non-invasive method such as, for example, reverse ionotophoresis. In general, iontophoresis is the application of a small electric current to enhance the transport of both charged and polar, neutral compounds across the skin. Reverse iontophoresis is the term used to describe the process whereby molecules are extracted from the body to the surface of the skin in the presence of an electrical current. The negative charge of the skin at buffered pH causes it to be permselective to cations causing solvent flow towards the anode. This flow is the dominant force allowing movement of neutral molecules across the skin. This technology can be used in devices for non-invasive and continuous monitoring of compounds in interstitial fluid of individuals with disease. See, e.g., Rhee, et al., J. Korean Med. Sci. 22:70-73, 2007; Sieg, et al., Clin. Chem. 50:1383-1390, 2004; which are incorporated herein by reference).

### Drug Delivery Methods

A treatment regimen for treating a bone loss disease or bone loss disorder can include one or more follicle stimulating hormone (FSH) modulator, optionally in combination with replacement therapy that includes one or more steroid hormones or metabolites or modulators thereof. The at least one treatment regimen can be based on measurements of the cyclic physiological pre-disease levels of FSH, or steroid hormone levels, in the mammalian subject and on current cyclic levels of FSH, or steroid hormone levels, in the mammalian subject, or the at least one treatment regimen can be based on FSH levels, or steroid hormone levels in the population of female subjects or male subjects. In some aspects, the one or more FSH modulators are administered alone. In some aspects where the treatment regimen includes two or more FSH modulators, the FSH modulators can be administered as separate formulations or co-administered in the same formulation. In other aspects, the one or more FSH modulators are administered in combination with one or more steroid hormones or metabolites or modulators thereof, and/or one or more osteoporosis medications. Each component of the treatment regimen can be administered as separate formulations, co-administered in the same formulation, or combinations thereof.

A treatment regimen that includes one or more FSH modulators can be administered to a mammalian subject by a variety of methods, for example, via oral, parenteral, subcutaneous, intravenous, intramuscular, intraperitoneal, transdermal, transbuccal, intraocular, or intravaginal routes, e.g., by inhalation, intra-nasal spray, by depot injections, or by hormone implants. Pharmaceutical compositions including one or more FSH modulators or combinations thereof, and a suitable carrier can be solid dosage forms that include, but are not limited to, tablets, capsules, cachets, pellets, pills, powders and granules; topical dosage forms that include, but are not limited to, solutions, powders, fluid emulsions, fluid suspensions, semi-solids, ointments, pastes, creams, gels and jellies, and foams; and parenteral dosage forms that include, but are not limited to, solutions, suspensions, emulsions, and dry powders. The pharmaceutical compositions and delivery methods described herein are also applicable to the delivery of one or more steroid hormone and/or delivery of one or more osteoporosis medication.

The administration of a treatment regimen including one or more FSH modulators can constitute a single dose, multiple daily doses, multiple doses per day, continuous infusion and or time released dose. A cyclic, continuous or combination dosing regime can be used. For example, daily dosing with one or more FSH modulators, e.g., an FSH inhibitor and/or FSH receptor antagonist can be part of a 28 day cycle of drug administration that includes 21 to 24 days of daily dosing with the FSH inhibitor and/or FSH receptor antagonist, followed by 4 to 7 days of dosing with a substantially reduced dose of the FSH inhibitor and/or FSH receptor antagonist or with a sugar pill or no dosing at all (“drug holiday”). During the 4 to 7 days of reduced or no levels of the FSH inhibitor and/or FSH receptor antagonist, the FSH levels can rise, inducing a spike in FSH levels that simulates pre-disease cycling of FSH levels. The treatment regimen can include multiple 28 day cycles over the course of months to years.

A treatment regimen including one or more FSH modulators can be administered orally using, for example, push-fit capsules made of gelatin or soft sealed capsules made of gelatin and a plasticizer such as glycerol or sorbitol. One or more FSH modular can be combined with fillers such as, e.g., lactose, binders such as, e.g., starches, and/or lubricants such as, e.g., talc or magnesium stearate and, optionally, stabilizers. In soft capsules, one or more FSH modulators can be dissolved or suspended in suitable liquids, such as fatty oils, liquid paraffin, or liquid polyethylene glycols. In addition, stabilizers can be added.

A treatment regimen including one or more FSH modulators can be administered by inhalation using an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount.

A treatment regimen including one or more FSH modulators can be formulated for parenteral administration by injection, e.g., by bolus injection or continuous infusion. In some aspect, one or more FSH modulators can be administered by continuous infusion subcutaneously over a period of about 15 minutes to about 24 hours. In some instances, continuous infusion can be done over the course of days and/or months. Compositions for injection can be presented in unit dosage form, e.g., in ampoules or in multi-dose containers, with an added preservative. The compositions can take such forms as suspensions, solutions or emulsions in oily or aqueous vehicles, and can contain agents such as suspending, stabilizing and/or dispersing agents.

### Transdermal Delivery Method

A treatment regimen for treating a bone loss disease or a bone loss disorder that includes one or more follicle-stimulating hormone (FSH) modulator, optionally including one or more steroid hormones or metabolites or modulators thereof, can be delivered through or across the skin of a subject using either passive or active transdermal delivery methods. Passive transdermal delivery methods utilize passive diffusion of agents across the skin and are exemplified by adhesive transdermal patches. In this aspect, a patch can be applied to the skin of a subject and one or more FSH modulators slowly and continuously diffuses out of the patch at a rate dictated by the formulation of the one or more FSH modulators and the composition of the patch.

In some aspects, a transdermal patch for administering one or more FSH modulators includes a non-permeable backing layer, a permeable surface layer, an adhesive layer, and a reservoir containing the drug composition. Examples of suitable materials can comprise the non-permeable backing layer and are known in the art of transdermal patch delivery. Materials for transdermal patch delivery include, but are not limited to, polyester film, such as high density polyethylene, low density polyethylene or composites of polyethylene; polypropylene; polyvinyl chloride, polyvinylidene chloride; ethylene-vinyl acetate copolymers; and the like. Examples of suitable permeable surface layer materials are also well known in the art of transdermal patch delivery, and any conventional material that is permeable to the one or more hormone to be administered, can be employed. Specific examples of suitable materials for the permeable surface layer include but are not limited to dense or microporous polymer films such as those comprised of polycarbonates, polyvinyl chlorides, polyamides, modacrylic copolymers, polysulfones, halogenated polymers, polychloroethers, acetal polymers, acrylic resins, and the like. See, e.g., U.S. Patent Publication 2008/0119449, which is incorporated herein by reference. Examples of suitable adhesives that can be coated on the backing layer to provide the adhesive layer are also known in the art and include, for example, pressure sensitive adhesives such as those comprising acrylic and/or methacrylic polymers. Specific examples of suitable adhesives include polymers of esters of acrylic or methacrylic acid (e.g., n-butanol, n-pentanol, isopentanol, 2-methyl butanol, 1-methyl butanol, 1-methyl pentanol, 3-methyl pentanol, 3-methyl pentanol, 3-ethyl butanol, isooctanol, n-decanol, or n-dodecanol esters thereof) alone or copolymerized with ethylenically unsaturated monomers such as acrylic acid, methacrylic acid, acrylamide, methacrylamide, N-alkoxymethyl acrylamides, N-alkoxymethyl methacrylamides, N-t-butylacrylamide, itaconic acid, vinyl acetate, N-branched C.sub.10-24 alkyl maleamic acids, glycol diacrylate, or mixtures of the foregoing; natural or synthetic rubbers such as silicon rubber, styrene-butadiene rubber, butyl-ether rubber, neoprene rubber, nitrile rubber, polyisobutylene, polybutadiene, and polyisoprene; polyurethane elastomers; vinyl polymers such as polyvinyl alcohol, polyvinyl ethers, polyvinyl pyrrolidone, and polyvinyl acetate; ureaformaldehyde resins; phenol formaldehyde resins; resorcinol formaldehyde resins; cellulose derivatives such as ethyl cellulose, methyl cellulose, nitrocellulose, cellulose acetatebutyrate, and carboxymethyl cellulose, and natural gums such as guar, acacia, pectin, starch, destria, gelatin, casein, and the like.

In some aspects, one or more FSH modulators can be administered by active transdermal delivery methods that utilize an energy source to increase the flux of the one or more FSH modulators across the skin either by altering the barrier function of the skin (primarily the stratum corneum) or by increasing the energy of the hormone molecules. In this aspect, the amount of one or more FSH modulators delivered through the skin to the mammalian subject is proportional to the overall amount of energy applied.

Energy sources for use in active transdermal delivery include, but are not limited to, electrical (e.g., iontophoresis and electroporation), ultrasonic (phonophoresis, sonophoresis), magnetic (magnetophoresis), and thermal energies. See, e.g., Gordon, et al., “Transdermal Delivery: 4 Myths about transdermal drug deliver”, Drug Delivery Technology, 3(4): June 2003 which is incorporated herein by reference. For example, iontophoresis uses low voltage electrical current to drive ionized agents or drugs across the skin. An electric current flows from an anode to a cathode, with the skin completing the circuit and drives ionized molecules into the skin from a reservoir associated with the transdermal delivery device. By contrast, electroporation uses short electrical pulses of high voltage to create transient aqueous pores in the skin through which an agent or drug can be transported. Phonophoresis or sonophoresis uses low frequency ultrasonic energy to disrupt the stratum corneum. For example, studies provide enhanced systemic levels of topical dexamethasone when applied in combination with ultrasound pulsed with an intensity of 1.0 W/cm² at a frequency of 3-MHz for 5 minutes. See, e.g., Saliba, et al., J. Athletic Training. 43:349-354, 2007, which is incorporated herein by reference. Thermal energy can be used to facilitate transdermal delivery by making the skin more permeable and by increasing the energy of drug molecules. In some aspect, one or more chemical permeation enhancer can be included. Examples of such enhancers include, but are not limited, to isopropyl myristate, bile salts, surfactants, fatty acids and derivatives, chelators, cyclodextrins or chitosan.

In some aspects, transdermal delivery of one or more FSH modulators can be facilitated using microporation induced by an array of microneedles. The microneedles can be hollow needles, solid-needles coated with one or more FSH modulators, dissolvable microneedles composed of one or more FSH modulators, or combinations thereof. Microneedles, when applied to the skin, painlessly create micropores in the stratum corneum without causing bleeding and lower the resistance to drug diffusion through the skin. The microneedles can be used to abrade or ablate the skin prior to transdermal transport of one or more FSH modulators. For example, a micro-array of heated hollow posts can be used to thermally ablate human skin in preparation for transdermal drug delivery by diffusion as described in U.S. Patent Application 2008/0045879, which is incorporated herein by reference. Alternatively, an array of microfine lances or microneedles can be designed to actively inject drug into the skin as described in Roxhed, et al., IEEE Transactions on Biomedical Engineering, 55:1063-1071, 2008, which is incorporated herein by reference.

In some aspects, transdermal delivery of one or more FSH modulators, optionally in combination with one or more steroid hormones or metabolites or modulators thereof, facilitated by an energy source can be combined with a method that perforates or abrades the skin of a subject. For example, a transdermal delivery method can combine iontophoresis with one or more microprojections that perforate the skin and enhance penetration and delivery of an agent as described, for example, in U.S. Pat. No. 6,835,184 and U.S. Patent Application 2006/0036209, which are incorporated herein by reference. In another example, an energy source such as iontophoresis or electroporation can be combined with electrically-induced ablation of skin cells as described in U.S. Pat. No. 7,113,821, which is incorporated herein by reference.

In further aspects, one or more FSH modulators, optionally in combination with one or more steroid hormones or metabolites or modulators thereof, can be delivered to a subject by a transdermal delivery method by one or more functional modes, for example, completely automatic with a preset dosage regimen, controlled by the subject or other individual, or automatically controlled by a feedback mechanism based on the normal physiological level of FSH. For example, a preset dosage regimen of one or more FSH modulators can be administered to a subject to reduce the bioactivity or bioavailability of endogenous FSH and bring the latter to physiologically normal or pre-disease levels. A transdermal delivery system can be designed that automatically times the activation and deactivation of an electrical power supply, for example, for delivery and cessation of delivery of a drug at a variable controlled rate at preset or preprogrammed time intervals as described in U.S. Pat. No. 5,224,928, which is incorporated herein by reference. In some aspects, the pre-set dosage regimen can be programmed into the transdermal delivery method at the time of manufacture. In a further aspect, the transdermal delivery method can have a removable computer interface component that can be externally programmed for a specific drug delivery regimen and reinserted into the device such as described in U.S. Pat. No. 6,539,250, which is incorporated herein by reference.

In a further aspect, one or more FSH modulators can be delivered to a subject by a transdermal delivery method, parenteral delivery method, or oral or nasal delivery method by one or more functional modes, for example, automatically controlled by a feedback mechanism based on the normal physiological level of FSH.

In some aspects, the delivery of one or more FSH modulators by a transdermal delivery method can be controlled either by the subject or other individual, for example, a healthcare provider, using on/off and/or high/low settings. See, e.g., U.S. Pat. No. 5,224,927, which is incorporated herein by reference. In some instances, it can be of benefit to limit or regulate the number of doses allowed by the subject. The transdermal delivery method can incorporate a preprogrammed number of doses allowed during a given time period.

### Implantable Delivery Method

A treatment regimen for treating a bone loss disease or a bone loss disorder that includes one or more follicle-stimulating hormone (FSH) modulators, optionally in combination with one or more steroid hormones or metabolites or modulators thereof, can be delivered systemically and/or to a specific site of action using an implantable delivery method. In some aspect, an implantable delivery method can incorporate a polymer or other matrix that allows for passive and slow release of one or more FSH modulators. For example, a biologically active compound can be formulated with a solid hydrophilic polymer that swells by osmotic pressure after implantation, allowing interaction with a solubilizing agent and release of the biologically active compound through a non-porous rate-controlling membrane. See, e.g., U.S. Pat. No. 5,035,891, which is incorporated herein by reference. In other aspects, one or more FSH modulators can be delivered using an implantable delivery method that includes an infusion pump that actively moves the one or more FSH modulators from an associated reservoir into a subject. A variety of pumps can be incorporated into an implantable delivery method, for example, a piston pump, rotary vane pump, osmotic pump, Micro Electro Mechanical Systems (MEMS) pump, diaphragm pump, peristaltic pump, or solenoid piston pump. In some aspects, the infusion pump can be a vapor-pressure powered pump in which a fluorocarbon charging fluid such as freon is used to drive the pump as a vapor-liquid mixture at normal body temperature and atmospheric pressure. In a further aspect, the infusion pump can be a battery operated peristaltic pump. The latter is exemplified by an intrathecal drug delivery device in which an infusion pump with a controllable receiver unit is implanted under skin and a catheter is fed into the target site, in this case the spine. See, e.g., Belverud, Neurotherapeutics. 5:114-122, 2008, which is incorporated herein by reference. An external device can be used to wirelessly control the pump. In some aspects, the reservoir associated with the pump can be refillable via percutaneous injection.

A treatment regimen that includes one or more FSH modulators configured to reduce bioactivity or bioavailability of FSH and to approach a cyclic physiological pre-disease level of in a subject can be delivered using an implantable delivery method that incorporates a MEMS (Micro Electro Mechanical Systems) fabricated microchip. Examples of MEMS and/or microfabricated devices for potential delivery of a therapeutic agent are described in U.S. Pat. Nos. 5,993,414; 6,454,759; and 6,808,522, which are incorporated herein by reference. The MEMS implantable delivery method can have one or more microfabricated drug reservoirs such as, for example, microparticle reservoirs, silicon microarray reservoirs, and/or polymer microreservoirs as described by Grayson, et al., Proceedings of the IEEE, 92: 6-21, 2004, which is incorporated herein by reference. Microparticles fabricated from silicon can contain an internal space that is loaded with drug using a microinjector and capped, e.g., with a slow dissolving gelatin or starch. Polymer microreservoirs can be fabricated by micromolding poly(dimethylsiloxane) or by patterning in multilayer poly(D-lactic acid) and (vinyl alcohol), for example. In some instances, the polymer microreservoirs can be capped with polymers that degrade at various rates in vivo depending upon the length of the polymer, allowing for controlled release of multiple doses.

In some aspects, an array of microreservoirs on a microchip can be used in which each dose of one or more FSH modulators is contained within separate reservoirs and capped by an environmentally sensitive material. For example, the microreservoirs can be capped with a gold membrane that is weakened and ruptured by electrochemical dissolution in response to application of an anode voltage to the membrane in the presence of chloride ions, resulting in release of drug as described in U.S. Pat. No. 5,797,898 and in Prescott, et al., Nat. Biotech., 24:437-438, 2006, which are incorporated herein by reference. Alternatively, the microreservoirs can be capped by a temperature sensitive material that ruptures in response to selective application of heat to one or more of the reservoirs as described in U.S. Pat. No. 6,669,683, which is incorporated herein by reference. Wireless induction of a voltage or thermal trigger to a given reservoir of the microarray enable on-demand release of one or more steroid hormones when activated by a subject or other individual. In other aspects, the microchip array can incorporate a sensor component that signals release of one or more FSH modulators by a closed-loop mechanism in response to a chemical or physiological state. See, e.g., U.S. Pat. No. 6,976,982, which is incorporated herein by reference.

In some instances, the implantable delivery method can incorporate a natural and/or synthetic stimulus-responsive hydrogel or polymer that changes confirmation rapidly and reversibly in response to environmental stimuli such as, for example, temperature, pH, ionic strength, electrical potential, light, magnetic field or ultrasound. See, e.g., Stubbe, et al., Pharmaceutical Res., 21:1732-1740, 2004, which is incorporated herein by reference. Examples of polymers are described in U.S. Pat. Nos. 5,830,207; 6,720,402; and 7,033,571, which are incorporated herein by reference. In some aspects, the one or more FSH modulators to be delivered by the implantable delivery method can be dissolved or dispersed in the hydrogel or polymer. Alternatively, a hydrogel and/or other stimulus-responsive polymer can be incorporated into an implantable delivery device. For example, a hydrogel or other polymer or other smart material can be used as an environmentally sensitive actuator to control flow of a therapeutic agent out of an implantable device as described in U.S. Pat. Nos. 6,416,495; 6,571,125; and 6,755,621, which are incorporated herein by reference. An implantable delivery device can incorporate a hydrogel or other polymer that modulates delivery of one or more FSH modulators in response to environmental conditions.

In some aspects, the implantable delivery method can be non-programmable, delivering a predetermined dosage of one or more FSH modulators. For example, one or more FSH modulators can be administered using continuous infusion. Alternatively, the dosage of a one or more FSH modulators can be predetermined to deliver a dose based on a timing mechanism associated with the implantable device. The timing device can be linked to a defined dosing cycle of 21 to 35 days that simulates a menstrual cycle and delivers appropriate levels of one or more FSH modulators during the 21 to 35 day treatment cycle to approach a target cyclic physiological pre-disease level of FSH. Alternatively, the implantable device can be programmable, having on/off and/or variable delivery rates based on either external or internal control. External control can be mediated by manual manipulation of a hand-operated pulsative pump with one-way valves associated with a delivery device implanted near the surface of the skin, for example. Alternatively, external control can be mediated by remote control through an electromagnetic wireless signal such as, for example, infrared or radio waves that are able to trigger an electrical stimulus within the implanted device. Examples of remote control drug delivery devices are described in U.S. Pat. Nos. 5,928,195; 6,454,759; and 6,551,235, which are incorporated herein by reference. One or more FSH modulators can be delivered by continuous infusion in response to an “on” trigger and stopped in response to an “off” trigger, for example. Alternatively, FSH modulators can be delivered as a microbolus, for example, in response to an “on” trigger as described in U.S. Pat. No. 6,554,822, which is incorporated herein by reference. In some aspects, external control can be initiated by a caregiver. Alternatively, a subject can initiate delivery of one or more FSH modulators. The system can have a built in mechanism to limit the number of allowable doses by a subject and/or caregiver in a given time frame as described, for example, in U.S. Pat. No. 6,796,956, which is incorporated herein by reference.

An implantable device for delivery of one or more FSH modulators, optionally in combination with one or more steroid hormones or metabolites or modulators thereof, can be powered by a standard lithium battery. In some instances, the battery can be rechargeable. For example, a battery associated with an implantable device can be recharged transcutaneously via inductive coupling from an external power source temporarily positioned on or near the surface of the skin as described in U.S. Pat. No. 7,286,880, which is incorporated herein by reference. Alternatively, the energy source for an implantable device can come from within the subject. For example, an implantable device can be powered by conversion of thermal energy from the subject into an electrical current as described in U.S. Pat. No. 7,340,304, which is incorporated herein by reference. Other methods of recharging or directly driving a battery associated with an implantable device include but are not limited to electromagnetic energy transmission, piezoelectric power generation, thermoelectric devices, ultrasonic power motors, radio frequency recharging and optical recharging methods as described in Wei & Liu. Front. Energy Power Eng. China 2:1-13, 2008, which is incorporated herein by reference.

### Pharmaceutical Formulations

A method for treating a bone loss disease or a bone loss that includes a treatment regimen configured to and in an amount sufficient to reduce the bioactivity or bioavailability of follicle stimulating hormone (FSH) in a subject using an FSH modulator in combination with a pharmaceutical formulation. The pharmaceutical formulation that includes one or more FSH modulator, optionally in combination with one or more steroid hormones or metabolites or modulators thereof, can be formulated neat or can be combined with one or more acceptable carriers, diluents, excipients, and/or vehicles such as, for example, buffers, surfactants, preservatives, solubilizing agents, isotonicity agents, and stabilizing agents as appropriate. A “pharmaceutically acceptable” carrier, for example, can be approved by a regulatory agency of the state and/or Federal government such as, for example, the United States Food and Drug Administration (US FDA) or listed in the U.S. Pharmacopeia or other generally recognized pharmacopeia for use in animals, and more particularly in humans. Conventional formulation techniques generally known to practitioners are described in Remington: The Science and Practice of Pharmacy, 20^{th} Edition, Lippincott Williams & White, Baltimore, Md. (2000), which is incorporated herein by reference.

Acceptable pharmaceutical carriers include, but are not limited to, the following: sugars, such as lactose, glucose and sucrose; starches, such as corn starch and potato starch; cellulose, and its derivatives, such as sodium carboxymethyl cellulose, ethyl cellulose, cellulose acetate, and hydroxymethylcellulose; polyvinylpyrrolidone; cyclodextrin and amylose; powdered tragacanth; malt; gelatin, agar and pectin; talc; oils, such as mineral oil, polyhydroxyethoxylated castor oil, peanut oil, cottonseed oil, safflower oil, sesame oil, olive oil, corn oil and soybean oil; polysaccharides, such as alginic acid and acacia; fatty acids and fatty acid derivatives, such as stearic acid, magnesium and sodium stearate, fatty acid amines, pentaerythritol fatty acid esters; and fatty acid monoglycerides and diglycerides; glycols, such as propylene glycol; polyols, such as glycerin, sorbitol, mannitol and polyethylene glycol; esters, such as ethyl oleate and ethyl laurate; buffering agents, such as magnesium hydroxide, aluminum hydroxide and sodium benzoate/benzoic acid; water; isotonic saline; Ringer's solution; ethyl alcohol; phosphate buffer solutions; other non-toxic compatible substances employed in pharmaceutical compositions.

A treatment regimen including a pharmaceutical formulation of one or more FSH modulators can be formulated in a pharmaceutically acceptable liquid carrier. The liquid carrier or vehicle can be a solvent or liquid dispersion medium comprising, for example, water, saline solution, ethanol, a polyol, vegetable oils, nontoxic glyceryl esters, and suitable mixtures thereof. The solubility of a chemical blocking agent can be enhanced using solubility enhancers such as, for example, water; diols, such as propylene glycol and glycerol; mono-alcohols, such as ethanol, propanol, and higher alcohols; DMSO (dimethylsulfoxide); dimethylformamide, N,N-dimethylacetamide; 2-pyrrolidone, N-(2-hydroxyethyl) pyrrolidone, N-methylpyrrolidone, 1-dodecylazacycloheptan-2-one and other n-substituted-alkyl-azacycloalkyl-2-ones and other n-substituted-alkyl-azacycloalkyl-2-ones (azones). The proper fluidity can be maintained, for example, by the formation of liposomes, by the maintenance of the necessary particle size in the case of dispersions or by the use of surfactants. One or more antimicrobial agent can be included in the formulation such as, for example, parabens, chlorobutanol, phenol, sorbic acid, and/or thimerosal to prevent microbial contamination. In some instances, it may be preferable to include isotonic agents such as, for example, sugars, buffers, sodium chloride or combinations thereof.

A treatment regimen including a pharmaceutical formulation of one or more FSH modulators, optionally in combination with one or more steroid hormones or metabolites or modulators thereof, for reducing the bioactivity or bioavailability of FSH and to approach a target cyclic physiological pre-disease level of FSH can be formulated for transdermal delivery. For example, water-insoluble, stratum corneum-lipid modifiers such as for example 1,3-dioxanes, 1,3-dioxolanes and derivatives thereof, 5-, 6-, 7-, or 8-numbered lactams (e.g., butyrolactam, caprolactam), morpholine, cycloalkylene carbonate have been described for use in transdermal iontophoresis. See, e.g., U.S. Pat. No. 5,527,797, which is incorporated herein by reference. Other suitable penetration-enhancing agents include but are not limited to ethanol, hexanol, cyclohexanol, polyethylene glycol monolaurate, azacycloalkan-2-ones, linoleic acid, capric acid, lauric acid, neodecanoic acid hexane, cyclohexane, isopropylbenzene; aldehydes and ketones such as cyclohexanone, acetamide; N,N-di(lower alkyl)acetamides such as N,N-diethylacetamide, N,N-dimethyl acetamide; N-(2-hydroxyethyl)acetamide; esters such as N,N-di-lower alkyl sulfoxides; essential oils such as propylene glycol, glycerine, isopropyl myristate, and ethyl oleate; salicylates; and mixtures of any of the above. See, e.g., U.S. Patent Publication 2008/0119449).

In some instances, a treatment regimen including a pharmaceutical formulation of one or more FSH modulators for reducing the bioactivity or bioavailability of FSH, optionally in combination with one or more steroid hormones or metabolites or modulators thereof, configured to approach a target cyclic physiological pre-disease level of FSH can be formulated in a dispersed or dissolved form in a hydrogel or polymer associated with, for example, an implantable or a transdermal delivery method. Examples of hydrogels and/or polymers include but are not limited to gelled and/or cross-linked water swellable polyolefins, polycarbonates, polyesters, polyamides, polyethers, polyepoxides and polyurethanes such as, for example, poly(acrylamide), poly(2-hydroxyethyl acrylate), poly(2-hydroxypropyl acrylate), poly(N-vinyl-2-pyrrolidone), poly(n-methylol acrylamide), poly(diacetone acrylamide), poly(2-hydroxylethyl methacrylate), poly(allyl alcohol). Other suitable polymers include but are not limited to cellulose ethers, methyl cellulose ethers, cellulose and hydroxylated cellulose, methyl cellulose and hydroxylated methyl cellulose, gums such as guar, locust, karaya, xanthan gelatin, and derivatives thereof. For iontophoresis, for example, the polymer or polymers can include an ionizable group such as, for example, (alkyl, aryl or aralkyl) carboxylic, phosphoric, glycolic or sulfonic acids, (alkyl, aryl or aralkyl) quaternary ammonium salts and protonated amines and/or other positively charged species as described in U.S. Pat. No. 5,558,633, which is incorporated herein by reference.

Information regarding formulation of FDA approved steroid hormones, or metabolites, modulators, or analogs thereof can be found in the package insert and labeling documentation associated with each approved agent. A compendium of package inserts and FDA approved labeling can be found in the Physician's Desk Reference. Alternatively, formulation information for approved chemical blocking agents can be found on the internet at websites, for example, www.drugs.com and www.rxlist.com. For example, ganirelix (Orgalutran®) and cetrorelix (Cetrotide®) synthetic decapeptide which is a GnRH antagonist that can be used to reduce serum levels of FSH, contains active drug, calcium phosphate tribasic, hydroxypropyl cellulose, microcrystalline cellulose, powdered cellulose, hypromellose, lactose monohydrate, magnesium stearate, polyethylene glycol, sucrose, and titanium dioxide. For those FSH modulators or steroid hormones or metabolites, modulators, or analogs thereof that do not currently have a formulation appropriate for use in any of the delivery methods described above, an appropriate formulation can be determined empirically and/or experimentally using standard practices. The pharmaceutical compositions are generally formulated as sterile, substantially isotonic and in full compliance with all Good Manufacturing Practice (GMP) regulations of the U.S. Food and Drug Administration.

### Device for Monitoring FSH Levels and Administering a Treatment Regimen Including One or More FSH Modulators

A method for treating a bone loss disease or disorder in a mammalian subject is disclosed that includes providing a device configured to communicate with at least a portion of the peripheral blood of a subject. Optionally, the device is further configured to communicate with at least a portion of one or more other bodily fluids of the subject including but not limited to urine, saliva, sweat, semen, vaginal excretions. The device includes one or more sensors configured to detect one or more hormones in the peripheral blood a subject. The device further includes a controller in communication with the sensor, a means for modulating one or more hormones responsive to the controller, the controller configured to adjust the modulating means to administer at least one FSH modulator, optionally in combination with one or more steroid hormones or metabolites or modulators thereof, to achieve a target cyclic pre-disease level of the one or more FSH or steroid hormones in the peripheral blood of the subject. The target value of the one or more hormones approaches a cyclic physiological pre-disease level of the one or more hormones in the peripheral blood of the subject.

Sensors

The device includes one or more sensors for qualitatively and/or quantitatively measuring one or more hormones in the peripheral blood of a subject. In some aspects, the device includes one or more sensors for sensing the levels of follicle stimulating hormone (FSH), optionally in combination with one or more steroid hormones or metabolites or modulators thereof. In other aspects, the device includes one or more sensors for sensing the levels of one or more steroid hormones, e.g., estradiol, progesterone, and/or testosterone. Optionally, the device further includes one or more sensors for sensing the levels of one or more markers of bone metabolism and/or bone health, e.g., blood calcium levels, parathyroid hormone, bone-specific alkaline phosphatase, osteocalcin, tartrate-resistant acid phosphatase-5b (TRAP), N-telopeptide of type I collagen (NTx), C-telopeptide of type I collagen (CTx), deoxypyridinoline (DPD), pyridinium crosslinks, vitamin D levels, inhibin A, inhibin B, or combinations thereof.

The one or more sensors can include, but are not limited to, a biosensor, a chemical sensor, a physical sensor, an optical sensor, or combinations thereof. The one or more sensors can include one or more recognition elements that recognize one or more hormones. The interaction of one or more hormones with one or more sensors results in one or more detectable signals. Preferably the one or more sensors measure in real-time the levels of one or more hormones in the peripheral blood of a subject.

The one or more recognition elements that can identify one or more hormones in the peripheral blood of a subject include, but are not limited to, antibodies, antibody fragments, peptides, oligonucleotides, DNA, RNA, aptamers, protein nucleic acids proteins, viruses, enzymes, receptors, bacteria, cells, cell fragments, inorganic molecules, organic molecules, synthetic recognition elements, or combinations thereof. The one or more recognition elements can be associated with a substrate integrated into the one or more sensors.

The one or more sensors for sensing one or more hormones can incorporate one or more recognition elements and one or more measurable fluorescent signals. In some aspects, one or more hormones in the peripheral blood of a subject are captured by one or more recognition elements and further react with one or more fluorescent second elements. The fluorescence associated with the captured one or more hormones can be measured using fluorescence spectroscopy. Alternatively, the fluorescence signal can be detected using at least one charged-coupled device (C CD) and/or at least one complimentary metal-oxide semiconductor (CMOS).

In some aspects, the one or more sensors can use Förster or fluorescence resonance energy transfer (FRET) to sense one or more hormones in the peripheral blood of a subject. FRET is a distance-dependent interaction between the electronic excited states of two dye molecules in which excitation is transferred from a donor molecule to an acceptor molecule without emission of a photon. In some aspects, interaction of a donor molecule with an acceptor molecule can lead to a shift in the emission wavelength associated with excitation of the acceptor molecule. In other aspects, interaction of a donor molecule with an acceptor molecule can lead to quenching of the donor emission. The one or more recognition elements associated with the one or more sensors can include at least one donor molecule and at least one acceptor molecule. Binding of one or more hormones to the recognition element can result in a conformation change in the recognition element, leading to changes in the distance between the donor and acceptor molecules and changes in measurable fluorescence. The recognition element can be a cell, an antibody, an aptamer, a receptor or any other molecule that changes conformation or signaling in response to binding one or more hormone.

A variety of donor and acceptor fluorophore pairs can be considered for FRET associated with the recognition element including, but not limited to, fluorescein and tetramethylrhodamine; IAEDANS and fluorescein; fluorescein and fluorescein; and BODIPY FL and BODIPY FL. A number of Alexa Fluor (AF) fluorophores (Molecular Probes-Invitrogen, Carlsbad, Calif., USA) can be paired with other AF fluorophores for use in FRET. Some examples include, but are not limited, to AF 350 with AF 488; AF 488 with AF 546, AF 555, AF 568, or AF 647; AF 546 with AF 568, AF 594, or AF 647; AF 555 with AF594 or AF647; AF 568 with AF6456; and AF594 with AF 647.

The cyanine dyes Cy3, Cy5, Cy5.5 and Cy7, that emit in the red and far red wavelength range (>550 nm), offer a number of advantages for FRET-based detection systems. Their emission range is such that background fluorescence is often reduced and relatively large distances (>100 Å) can be measured as a result of the high extinction coefficients and good quantum yields. For example, Cy3, that emits maximally at 570 nm and Cy5, that emits at 670 nm, can be used as a donor-acceptor pair. When the Cy3 and Cy5 are not proximal to one another, excitation at 540 nm results only in the emission of light by Cy3 at 590 nm. In contrast, when Cy3 and Cy5 are brought into proximity by a conformation change in an aptamer, antibody, or receptor, for example, excitation at 540 nm results in an emission at 680 nm. Semiconductor quantum dots (QDs) with various excitation/emission wavelength properties can also be used to generate a fluorescence based sensor.

Quenching dyes can be used as part of the binder element to quench the fluorescence of visible light-excited fluorophores. Examples include, but are not limited, to DABCYL, the non-fluorescing diarylrhodamine derivative dyes QSY 7, QSY 9 and QSY 21 (Molecular Probes, Carlsbad, Calif., USA), the non-fluorescing Black Hole Quenchers BHQ0, BHQ1, BHQ2, and BHQ3 (Biosearch Technologies, Inc., Novato, Calif., USA) and Eclipse (Applera Corp., Norwalk, Conn., USA). A variety of donor fluorophore and quencher pairs can be considered for FRET associated with the recognition element including, but not limited to, fluorescein with DABCYL; EDANS with DABCYL; or fluorescein with QSY 7 and QSY 9. In general, QSY 7 and QSY 9 dyes efficiently quench the fluorescence emission of donor dyes including blue-fluorescent coumarins, green- or orange-fluorescent dyes, and conjugates of the Texas Red and Alexa Fluor 594 dyes. QSY 21 dye efficiently quenches all red-fluorescent dyes. A number of the Alexa Fluor (AF) fluorophores (Molecular Probes-Invitrogen, Carlsbad, Calif., USA) can be paired with quenching molecules as follows: AF 350 with QSY 35 or DABCYL; AF 488 with QSY 35, DABCYL, QSY7 or QSY9; AF 546 with QSY 35, DABCYL, QSY7 or QSY9; AF 555 with QSY7 or QSY9; AF 568 with QSY7, QSY9 or QSY21; AF 594 with QSY21; and AF 647 with QSY 21.

The one or more sensor for sensing one or more hormones can use the technique of surface plasmon resonance (for planar surfaces) or localized surface plasmon resonance (for nanoparticles). Surface plasmon resonance involves detecting changes in the refractive index on a sensor surface in response to changes in molecules bound on the sensor surface. The surface of the sensor can be a glass support or other solid support coated with a thin film of metal, for example, gold. The sensor surface can further carry a matrix to which is immobilized one or more recognition elements that recognize one or more hormones. The one or more recognition elements that recognize one or more hormones can be antibodies or fragments thereof, oligonucleotide or peptide based aptamers, receptors of inflammatory mediators or fragments thereof, artificial binding substrates formed by molecular imprinting, or any other examples of molecules and or substrates that bind hormones. In some aspects, as blood or blood components from the subject pass by the sensor surface, one or more hormones can interact with one or more recognition elements on the sensor surface. The sensor is illuminated by monochromatic light. Resonance occurs at a specific angle of incident light. The resonance angle depends on the refractive index in the vicinity of the surface, which is dependent upon the concentration of molecules on the surface. An example of instrumentation that uses surface plasmon resonance is the BIACORE system (Biacore, Inc.—GE Healthcare, Piscataway, N.J.) that includes a sensor microchip, a laser light source emitting polarized light, an automated fluid handling system, and a diode array position sensitive detector. See, e.g., Raghavan & Bjorkman Structure 3:331-333, 1995, which is incorporated herein by reference.

The one or more sensors can be one or more label-free optical biosensors that incorporate other optical methodologies, e.g., interferometers, waveguides, fiber gratings, ring resonators, and photonic crystals. See, e.g., Fan, et al., Anal. Chim. Acta 620:8-26, 2008, which is incorporated herein by reference. For example, reflectometric interference spectroscopy can be used to monitor in real-time the interaction of the antigen with it's respective antibody. See, e.g., Piehler & Schreiber, Anal. Biochem. 289:173-186, 2001, which is incorporated herein by reference.

The one or more sensors for sensing one or more hormones can be one or more microcantilevers. A microcantilever can act as a biological sensor by detecting changes in cantilever bending or vibrational frequency in response to binding of one or more hormones to the surface of the sensor. In some aspects the sensor can be bound to a microcantilever or a microbead as in an immunoaffinity binding array. In other aspects, a biochip can be formed that uses microcantilever bi-material formed from gold and silicon, as sensing elements. See, e.g. Vashist J. Nanotech Online 3:DO: 10.2240/azojono0115, 2007, which is incorporated herein by reference. The gold component of the microcantilever can be coated with one or more recognition elements that upon binding one or more hormones causes the microcantilever to deflect. Aptamers or antibodies specific for one or more hormones can be used to coat microcantilevers. See, e.g., U.S. Pat. No. 7,097,662, which is incorporated herein by reference. The one or more sensor can incorporate one or more methods for microcantilever deflection detection including, but not limited to, piezoresistive deflection detection, optical deflection detection, capacitive deflection detection, interferometry deflection detection, optical diffraction grating deflection detection, and charge coupled device detection. In some aspects, the one or more microcantilever can be a nanocantilever with nanoscale components. The one or more microcantilevers and/or nanocantilevers can be arranged into arrays for detection of one or more hormones. Both microcantilevers and nanocantilevers can find utility in microelectomechnical systems (MEMS) and/or nanoelectomechnical systems (NEMS) associated with an implantable or external device.

The one or more sensor for sensing one or more hormones can be a field effect transistor (FET) based biosensor. In this aspect, a change in electrical signal is used to detect the interaction of one or more analytes with one or more components of the sensor. See, e.g., U.S. Pat. No. 7,303,875, which is incorporated herein by reference.

The one or more sensors for sensing one or more hormones can incorporate electrochemical impedance spectroscopy. Electrochemical impedance spectroscopy can be used to measure impedance across a natural and/or artificial lipid bilayer. The sensor can incorporate an artificial bilayer that is tethered to the surface of a solid electrode. One or more receptor can be embedded into the lipid bilayer. The one or more receptors can be ion channels that open and close in response to binding of a specific analyte. The open and closed states can be quantitatively measured as changes in impedance across the lipid bilayer. See, e.g., Yang, et al., IEEE SENSORS 2006, EXCO, Daegu, Korea/Oct. 22-25, 2006, which is incorporated herein by reference.

The one or more sensors for sensing one or more hormones can be cells that include one or more binding elements that when bound to one or more hormones induces a measurable or detectable change in the cells. In some aspect, the cells can emit a fluorescent signal in response to interacting with one or more hormones. For example, a bioluminescent bioreporter integrated circuit can be used in which binding of a ligand to a cell induces expression of reporter polypeptide linked to a luminescent response. See, e.g., U.S. Pat. No. 6,673,596, Durick & Negulescu Biosens. Bioelectron. 16:587-592, 2001, which are incorporated herein by reference. In other aspects, the one or more cells can emit an electrical signal in response to interacting with one or more hormones. In a further aspect, an implantable biosensor can be used which is composed of genetically-modified cells that responded to ligand binding by emitting a measurable electrical signal. See U.S. Patent Application 2006/0234369 A1; which is incorporated herein by reference.

The device can further include one or more sensors for sensing one or more physiological parameters in the subject. Examples of physiological parameters include but are not limited to body temperature, respiration rate, pulse, blood pressure, edema, oxygen saturation, pathogen levels, or toxin levels. Additional sensors for use in the device include but are not limited to biosensors, blood volume pulse sensors, conductance sensors, electrochemical sensors, fluorescence sensors, force sensors, heat sensors (e.g., thermistors, thermocouples, and the like), high resolution temperature sensors, differential calorimeter sensors, optical sensors, goniometry sensors, potentiometer sensors, resistance sensors, respiration sensors, sound sensors (e.g., ultrasound), Surface Plasmon Band Gap sensor (SPRBG), physiological sensors, surface plasmon sensors, and the like. Further non-limiting examples of sensors include affinity sensors, bioprobes, biostatistics sensors, enzymatic sensors, in-situ sensors (e.g., in-situ chemical sensor), ion sensors, light sensors (e.g., visible, infrared, and the like), microbiological sensors, microhotplate sensors, micron-scale moisture sensors, nanosensors, optical chemical sensors, single particle sensors, and the like. Further non-limiting examples of sensors include chemical sensors, cavitand-based supramolecular sensors, deoxyribonucleic acid sensors (e.g., electrochemical DNA sensors, and the like), supramolecular sensors, and the like. In an embodiment, at least one of the one or more sensors is configured to detect or measure the presence or concentration of FSH. Further examples of the one or more sensors include, but are not limited to, chemical transducers, ion sensitive field effect transistors (ISFETs), ISFET pH sensors, membrane-ISFET devices (MEMFET), microelectronic ion-sensitive devices, potentiometric ion sensors, quadruple-function ChemFET (chemical-sensitive field-effect transistor) integrated-circuit sensors, sensors with ion-sensitivity and selectivity to different ionic species, and the like.

Controller in Communication with and Responsive to a Sensor

The device further includes a controller that is in communication with and configured to be informed by the one or more sensors. The one or more sensors can transmit data to the controller regarding the detection or levels (relative or absolute) of one or more hormones, e.g., follicle stimulating hormone (FSH), optionally in combination with one or more steroid hormones or metabolites or modulators thereof, in the peripheral blood of a mammalian subject. The controller can be integrated into the device. Alternatively, the controller can be a separate component of the device that receives and transmits data and/or commands either with or without wires. For example, an implanted device can send data regarding the sensed levels of one or more hormones to an external controller through a wireless signal.

The controller can compare the input data regarding the one or more hormones in the peripheral blood of a subject with stored data regarding the time-history profile of one or more hormones, e.g., follicle stimulating hormone (FSH), estrogen, progesterone, and/or testosterone. The controller itself can include the stored data. Alternatively, the controller can have access to one or more remote databases that include the stored data. The stored data can be data regarding the subject's cyclic physiological pre-disease levels of one or more hormones. The stored data can further include the cyclic physiological levels of one or more hormones in age matched, normal or healthy subjects without a bone loss disease or disorder. The stored data can further include data regarding the level of one or more hormones in a subject at one or more previous time points, e.g., pre-disease, at diagnosis, at the initiation of treatment, and during treatment.

The controller assesses the most recently obtained input data with the stored data and is configured to controllably initiate steps to deliver at least one of a FSH modulator, optionally in combination with one or more steroid hormones or metabolites or modulators thereof, and/or an osteoporosis medication to the mammalian subject. In some aspects, the controller can release one or more FSH modulators from one or more reservoirs associated with the device. Alternatively, the controller can send data regarding the levels of one or more hormones in the peripheral blood of a subject to the subject, to one or more third party individuals such as a physician or other caregiver, to a computing device, or to a combination thereof. The subject and/or caregiver or computing device can choose to initiate steps to administer one or more FSH modulators to the subject.

Device Drug Delivery

In some aspects, all or part of the device is implanted into a mammalian subject. Examples of implantable devices include but are not limited to subdermal or subcutaneous devices (e.g., artificial pacemaker, long acting contraceptives, implantable microchips, nanostructures), luminal devices (e.g. endoscope robot), luminal traveling devices, by-pass devices, intracorporeal devices (e.g., stent, left ventricular assist device (LVAD)). See, e.g., US Patent Application 2009/0130017, US Patent Application 2009/0137866, US Patent Application 2009/0112191, US Patent Application 2009/0093807, US Patent Application 2008/0140057, Martel, et al., Applied Physics Lett. 90:114105, 2007, which are incorporated herein by reference. In some aspects, the device is a subcutaneous device that includes sensors as described herein for sensing the level of one or more hormone in a subject and a controller linked to an infusion pump for controllably releasing one or more FSH modulators to approach a target cyclic physiological level of FSH. In other aspects, the device is an intracorporeal, stent-like device that includes sensors as described herein for sensing the level of one or more hormones in a subject and a controller linked to a reservoir for controllably releasing one or more FSH modulators to approach a target cyclic physiological level of FSH in a subject.

In some aspects, all or part of the device is external to the mammalian subject. In some aspects, the device is in close proximity to the skin and is able to non-invasively sense the levels of one or more hormones in the circulation of a subject. Examples of methods for non-invasive sensing of blood complements include but are not limited to retinal imaging, near-infrared transmission spectroscopy, raman spectroscopy, optical coherence tomography, light scattering, photoacoustic spectroscopy, reverse iontophoresis. See, e.g., U.S. Pat. No. 6,477,394, U.S. Pat. No. 7,524,671, Burmeister & Arnold Clin. Chem. 45:1621-1627, 1999, Pickup, et al., BMJ 319:1289, 1999, Sieg, et al., Clin. Chem. 50:1383-1390, 2004, which are incorporated herein by reference. In a further aspect, the device is in close proximity to the skin and is able to extract a small sample of blood and/or other body fluid from a subject and sense the levels of one or more hormones using one or more sensors as described herein. In a further aspect, the device is an external device worn on the surface of a subject's skin and includes a sensor for non-invasive sensing of one or more hormones and a controller linked to a reservoir for controllable delivery of one or more FSH modulators into the subject. Controllable external delivery of one or more FSH modulators and/or steroid hormone and/or osteoporosis medication can include but is not limited to one or more of an infusion pump, a controllable transdermal patch, an ionophoresis system, an electroporation system, a series of one or more microneedles, an abrasion system linked to a dispensing reservoir, or combinations thereof.

### Assays for Identifying Modulators of Follicle Stimulating Hormone

Methods are described for identifying at least one or more novel modulators of follicle stimulating hormone. The follicle-stimulating hormone (FSH) modulator can be an inhibitor of FSH synthesis and/or secretion, an inhibitor of FSH binding activity, an inhibitor or antagonist of the FSH receptor, or combinations thereof.

One or more assay systems can be used to identify inhibitors of FSH synthesis and/or secretion. In some aspects, the assay system uses a primary cell culture system that naturally synthesizes and secretes FSH, for example, gonadotrophs isolated from the anterior pituitary. The one or more assay system can use monodispersed anterior pituitary cells isolated by dissection and digestion of the pituitary gland from a mammalian brain, e.g., a rat brain. Cells isolated in this manner are cultured and assayed for secretion of FSH in response to an activator of FSH synthesis and/or secretion, e.g., activin. See, e.g., Miyamoto, et al., J. Endocrinol. 161:375-382, 1999, which is incorporated herein by reference. In some aspects, the assay system for identifying antagonists of FSH synthesis and/or secretion uses an immortalized cell line that secretes FSH in response to activin, an example of which is the pituitary tumor gonadotroph cell line LβT2. See, e.g., Graham, et al., J. Endocrinol. 162:R1-R5, 1999, which is incorporated herein by reference. An assay system is devised using said cells to measure the ability of potential antagonists to inhibit activin-induced synthesis and/or secretion of FSH as measured by changes in FSH messenger RNA (mRNA) and/or changes in FSH polypeptide secreted into the cell culture medium. Changes in FSH messenger RNA can be monitored using any of a number methods including, but not limited to, quantitative polymerase chain reaction (PCR) amplification, microarray hybridization, northern analysis, ribonuclease protection assays, and the like. Changes in FSH polypeptide can be monitored using one or more of the assays systems described herein.

One or more assay systems can be used to identify modulators that neutralize FSH activity by binding to FSH and otherwise preventing it from binding to an FSH receptor. Examples of modulators for use in neutralizing FSH include, but are not limited to, antibodies, forms of soluble FSH receptor and other FSH binding proteins, or mimetics thereof. In some aspects, a binding assay is used to identify modulators capable of neutralizing FSH. In an exemplary configuration, surface plasmon resonance (SPR) is used to assess affinity of FSH antibodies for FSH using Biacore SPR technology (from, e.g., GE Healthcare, Waukesha, Wis.) in which FSH is immobilized on a sensor chip with a thin gold surface layer and the binding affinity of FSH antibodies is measured based on changes in refractive index in response to changes in mass close to the sensor chip surface. See, e.g., Malmborg & Borrebaeck J. Immunol. Methods 183:7-13, 1995, which is incorporated herein by reference.

One or more assays systems can be used to identify antagonists of the FSH receptor including, but not limited to, competitive binding assays, signal transduction assays, resorption assays, and other biological assays. The response of the FSH receptor to FSH is compared to the response to FSH in the presence of a putative antagonist.

In some aspects, the one or more assays for identifying antagonists of FSH receptor activity can include competitive binding assays in which potential antagonists are screened for the ability to compete with FSH for binding to the FSH receptor. In some aspects, the competitive binding assay uses intact cells expressing the FSH receptor. The FSH receptor for use in the competitive binding assay system can be naturally expressed in a mammalian cell, e.g., in granulosa cells, Sertoli cells, or osteoclasts. Alternatively, all or part of the FSH receptor for use in the competitive binding assay system can be expressed in a suitable host cell line, for example, in Chinese Hamster Ovary (CHO) cells using standard molecular biology techniques. See, e.g., Gudermann, et al., Endocrinol. 135:2204-2213, 1994; U.S. Pat. No. 6,372,711; which are incorporated herein by reference. Other suitable host cells can be used for expressing the FSH receptor. In other aspects, the competitive binding assay uses all or part of fully or partially purified FSH receptor. For example, a preparation of cell membranes containing the FSH receptor can be isolated by lysis of FSH receptor containing cells. See, e.g., Schneyer, et al., Clin. Chem. 37:508-514, 1991, which is incorporated herein by reference. Alternatively, all or part of the FSH receptor can be isolated, purified and attached to a substrate, e.g., beads, matrix, or microtiter plates, for use in the competitive binding assay.

The binding of native FSH to the FSH receptor is assayed alone or in the presence of one or more putative antagonists that compete for binding to the receptor. In some aspects, the FSH is modified with a measurable label and in this instance, the binding efficiency of the putative antagonist is inversely proportional to the measured response. In other aspects, the putative antagonist is modified with a measurable label, and the binding efficiency of the putative FSH receptor antagonist is directly proportional to the measured response. FSH and/or one or more FSH receptor antagonists for use in the competitive binding assay with the FSH receptor can be labeled with an enzyme linked to a color reaction, bioluminescent and/or chemiluminescent chemical reaction, colloidal gold, radioisotopes, magnetic labels, fluorescent fluorophore, lanthanide chelates (e.g., europium(III), terbium(III), samarium(III), and dysprosium(III)), quantum dots, luminescent inorganic crystals, up-converting phosphors, fluorescent nanoparticles, plasmon resonant particles, or combinations thereof.

In some aspects, the assay system for identifying FSH receptor antagonist provides activity measurements of signal transduction and/or down stream signaling events that occur in response to FSH binding. For example, binding of FSH to the FSH receptor in granulosa cells in the ovary results in an increase in the second messenger, cyclic AMP (cAMP). The amount of cAMP generated in response to activation of the FSH receptor is attenuated in the presence of an FSH receptor antagonist that inhibits the activity of the FSH receptor. Cells containing the FSH receptor, e.g., granulosa cells, Sertoli cells, genetically modified cells, or other cells are screened against one or more putative antagonists in the presence of FSH and the resulting cAMP levels are measured. The potency of a putative antagonist is inversely proportional to the cAMP levels. Various methods are available for measuring changes in cAMP levels including but not limited to enzyme immunoassays, immunofluorescence assays, radioimmunoassays, chemiluminescence immunoassays.

In some aspects, the FSH receptor modulators can be identified using a transactivation assay system in which intracellular changes in cAMP are linked to a detectable colorimetric, fluorescent and/or bioluminescent readout. For example, the assay system can use a cell line, e.g., Chinese Hamster Ovary (CHO) cells, stably transfected with the FSH receptor and cotransfected with a cAMP responsive element (CRE)/promoter directing the expression of a firefly luciferase reporter gene. See, e.g., U.S. Patent Application 2004/0236109, which is incorporated herein by reference. The interaction of FSH with the FSH receptor causes an increase in cAMP and induces transactivation of the luciferase reporter construct. The luciferase signal can be quantified using a luminescence counter. Constructs for generating a luciferase-based biosensor can be generated using recombinant molecular biology techniques or are available from commercial sources (e.g., GloSensor™ cAMP Assay from Promega, Madison, Wis.). Other suitable reporter genes for this purpose include but are not limited to LacZ, alkaline phosphatase, and green fluorescent protein. This type of transactivation assay can be used to rapidly interrogate changes in the concentration of intracellular cAMP using a live cell, nonlytic assay format. This format enables direct screens for allosteric modulators of Gs- and Gi-coupled 7-transmembrane receptors and improved hit identification through multiple measurements. Other live-cell assay systems for cAMP can be used, for example, a fluorescence resonance energy transfer (FRET) based system in which cells are genetically modified to express to a cyan fluorescent protein-Epac-yellow fluorescent protein complex that fluoresces in the absence of cAMP but exhibits decreasing fluorescence as cAMP levels rise. See, e.g., Ponsioen, et al., EMBO Reports 5:1176-1180, 2004, which is incorporated herein by reference.

In some aspects, the assay system for identifying antagonists of the FSH receptor involves measurement of steroid hormones secreted from cells derived from mammalian gonads. For example, ovary granulosa cells and testes Sertoli cells secrete estradiol in response to FSH activation of the FSH receptors associated with these cells. In some aspects, an assay system is devised that uses granulosa cells and/or Sertoli cells isolated from a mammalian subject and cultured in the presence of FSH alone or in combination with an FSH receptor antagonist. The secretion of estradiol is measured in response to FSH activation of the FSH receptor with or without an antagonist and is inversely proportional to the efficacy of the antagonist. Estradiol in the culture medium can be measured using estradiol specific antibodies and any of the immunoassay detection systems described herein. See, e.g., U.S. Pat. No. 6,583,179, McDonald, et al., Mol. Endocrinol. 20:608-618, 2006, which are incorporated herein by reference.

In some aspects, the assay system for identifying FSH receptor antagonists includes measuring osteoclast differentiation and bone metabolism in osteoclast precursor cells, osteoclasts or other osteoclast-like cells. In one assay system, differentiation of osteoclast precursor cells into osteoclasts in response to FSH can be monitored by measuring changes in tartrate resistant acid phosphatase (TRAP). See, e.g., Sun, et al., Cell 125:247-260, 2006, which is incorporated herein by reference. Osteoclast precursor cells for use in the differentiation assay include, but are not limited to, primary cells (e.g., giant cell tumor (bone) derived cells, bone-marrow derived cells, mesenchymal cells, embryonic stem cells, hematopoietic stem cells) and various cell lines (e.g., RAW264.7 cells, RAW-C3 cells, FLG 29.1 cells). Other assays associated with osteoclast function can also be used to generate a screening assay and include but not limited to calcium flux, resorption pit formation, and collagen formation. See, e.g., Myers, et al., FEBS Letters 463:295-300, 1999; Blair, et al., J. Cell Biol. 102:1164-1172, 1986; Matsuoka, et al., J. Biomed. Mater. Res. 42:278-285, 1998, Susa, et al., J. Translational Med. 2:6, 2004, which are incorporated herein by reference.

### Kits

The invention provides kits comprising the compositions, e.g., nucleic acids, expression cassettes, vectors, cells, polypeptides (e.g., gonadotropins, FSH modulators, or steroid hormones or modulators thereof) and/or antibodies of the invention. The kits also can contain instructional material teaching the methodologies and uses of the invention, as described herein.

The methods and compositions are further described with reference to the following examples; however, it is to be understood that the methods and compositions are not limited to such examples.

### Example 1

### Follicle-Stimulating Hormone Inhibitor Treatment Regimen in Female Subject with Osteoporosis Disease

A treatment regimen is described that includes providing a follicle stimulating hormone (FSH) inhibitor for treating a perimenopausal or postmenopausal female subject diagnosed with an osteoporosis disease. The female subject is diagnosed with osteoporosis by her primary care physician and her endocrinologist. The diagnosis of osteoporosis is made based on the bone mineral density of the female subject's hip and spinal cord as measured by dual energy X-ray absorptiometry (DXA scan). The bone mineral density of the female subject is compared to that of healthy adult women 20-30 years of age and the resulting standard deviation or T score is lower than −2.5, indicative of osteoporosis as defined by the World Health Organization (WHO). In addition, one or more markers of bone turnover are assayed to confirm the osteoporosis diagnosis and for use in monitoring treatment efficacy. In this case study, bone specific alkaline phosphatase (BAP) is measured in the blood of the female subject by an immunoradiometric assay using a commercially available diagnostic kit (Hybritech Ostase®, Beckman Coulter, Fullerton, Calif.). Normal values of BAP in premenopausal women ranges from about 2.9 mg/L to about 14.5 mg/L and in postmenopausal women ranges from about 3.8 mg/L to about 22.6 mg/L. Elevated levels of BAP are indicative of increased bone turnover and resorption, hallmarks of osteoporosis.

The treatment regimen is based on the current and the pre-disease levels of FSH of the female subject, or on the pre-disease levels of FSH found generally in the female population. The current levels of FSH in the female subject are measured using an enzyme linked immunosorbent assay (ELISA). Blood is drawn from the female subject using standard venipuncture techniques into a glass vacuum tube (e.g., BD Vacutainer®, BD, Franklin Lakes, N.J.). Serum is isolated from the whole blood by allowing the blood to clot at 37° C. for 30-60 minutes. A long glass pipette or similar instrument is used to separate the clot from the sides of the glass tube. The serum is separated from the clot by decanting or pipetting the liquid into a new tube. The serum is spun at 3,000 rotations per minute (RPM) for 10 minutes to remove any remaining clots, blood cells or other insoluble material. Aliquots of the serum are assayed for FSH using a commercial ELISA diagnostic system as described by the manufacturer (from, e.g., BIOSERV Diagnostics, Rostock, Germany). The levels of FSH in the female subject range from about 25 U/liter to about 75 U/liter. These levels, in combination with the age of the subject, and the cessation of menses are indicative of a postmenopausal state. The current levels of FSH are compared with cyclic physiological pre-disease levels of FSH, the latter of which are part of the subject's medical record. Alternatively, cyclic physiological pre-disease levels of FSH can be determined from the general female population. The levels of FSH during the follicular phase of the cycle range from about 2.5 U/liter to about 10.2 U/liter. At the midcycle peak, the FSH levels rise to a range from about 3.4 U/liter to about 33.4 U/liter. During the luteal phase, the FSH levels fall and range from about 1.5 U/liter to about 9.1 U/liter.

A treatment regimen is designed that includes an FSH inhibitor. The treatment regimen includes an FSH inhibitor that is an antagonist of gonadotropin releasing hormone (GnRH). The GnRH antagonist inhibits the release of FSH from the anterior pituitary in a dose dependent manner. A GnRH antagonist that can be used to reduce serum levels of FSH is the synthetic decapeptide ganirelix (Orgalutran®). Ganirelix (250 micrograms in 500 microliters) is self administered once daily as a subcutaneous injection into the upper thigh or into the lower abdomen. Daily dosing with ganirelix is part of a 28 day cycle of drug administration. Ganirelix (250 micrograms) is administered once daily for 21 to 24 days of the 28 day cycle, followed by 4 to 7 days of subcutaneous dosing with saline or no dosing at all (“drug holiday”). During the 4 to 7 days in the absence of ganirelix, the FSH levels rise, inducing a spike in FSH levels that simulates pre-disease cycling of FSH levels, e.g., from about 3 U/liter to about 33 U/liter. The treatment regimen includes multiple 28 day cycles over the course of months to years.

The levels of FSH in the serum of the female subject are monitored on multiple days over the course of several 28 day treatment cycles to verify that the treatment regimen is lowering the FSH levels and that the periodic “drug holiday” is inducing a cyclic spike in FSH level. FSH can be measured in the serum of the female subject as described above using an ELISA system. If needed, treatment with the GnRH antagonist is adjusted either in terms of dosage or in terms of timing to achieve cyclic levels of FSH that approach the target cyclic physiological pre-disease levels. In addition, the efficacy of treatment with the FSH modulator on the osteoporosis disease can be monitored by reassessing the serum levels of one or more markers of bone resorption, e.g., BAP, and compared with serum levels measured prior to the initiation of the treatment regimen. A decrease in the serum BAP in response to the treatment regimen is indicative of decreased rate of bone turnover. Changes in bone mineral density as measured by a DXA scan or other imaging modality can also be used to monitor the efficacy of the treatment regimen.

### Example 2

### Follicle-Stimulating Hormone Receptor Antagonist Treatment Regimen in Female Subject with Oophorectomy and Osteoporosis Disease

A treatment regimen is described that includes a follicle stimulating hormone (FSH) receptor antagonist to treat a female subject who has undergone bilateral oophorectomy and has been diagnosed with osteoporosis. The female subject underwent a hysterectomy and bilateral salpingo-oophorectomy for non-malignant disease several years ago while still premenopausal. Oophorectomy in combination with hysterectomy in premenopausal women induces an immediate decline in estrogen and is linked to a higher risk of osteoporosis 3 to 6 years post-surgery as compared to similar aged women who undergo a hysterectomy alone. See, e.g., Aitken, et al., Br. Med. J. 2:325-328, 1973, which is incorporated herein by reference. The female subject is diagnosed with osteoporosis by her primary care physician and her endocrinologist. The diagnosis of osteoporosis is made based on bone mineral density of the female subject's wrist, heel, and/or finger as measured by peripheral dual energy x-ray absorptiometry (pDXA). The bone mineral density of the female subject is compared to that of a healthy adult women 20-30 years of age and the resulting standard deviation or T score is lower than −2.5, indicative of osteoporosis as defined by the World Health Organization. In addition, serum levels of the bone resorption marker tartrate resistant acid phosphatase 5b (TRAP) are assessed using a commercially available ELISA immunoassay system (e.g., BoneTRAP®, Immunodiagnostic Systems (IDS) Ltd., Tyne & Wear, UK) and are shown to be greater than 5 U/liter, above the upper normal limit for women (4.15 U/liter). Serum levels of bone alkaline phosphatase are also assessed as described herein.

The treatment regimen is based on the current and the pre-disease levels of FSH of the subject, or on the pre-disease levels of FSH found generally in the female population. The current levels of FSH in the female subject are measured using isolated serum and a chemiluminescence immunoassay (CLIA). In this assay system, one or more of the FSH antibodies in the assay are labeled with horseradish peroxidase that catalyzes oxidation of a luminol-based substrate resulting in a light-emitting enzymatic reaction. Light emission is detected using a luminometer and is directly proportional to the level of FSH in the serum sample. For the assay, blood is drawn from the female subject using standard venipuncture techniques into a glass vacuum tube in the absence of additives or anti-coagulants. Serum is isolated from the whole blood by allowing the blood to clot at 37° C. for 30-60 minutes. A long glass pipette or similar instrument is used to separate the clot from the sides of the glass tube. The serum is separated from the clot by decanting or pipetting the liquid into a new tube. The serum is spun at 3,000 rotations per minute (RPM) for 10 minutes to remove any remaining clots, blood cells or other insoluble material. Aliquots of the serum are assayed for FSH using a commercial CLIA diagnostic system as described by the manufacturer (e.g. FSH AccuLite® CLIA from Monobind, Inc., Lake Forest, Calif.). The levels of FSH in the female subject range from about 25 U/liter to about 75 U/liter. These levels, in combination with the oophorectomy are indicative of a postmenopausal state. Cyclic physiological pre-disease levels of FSH can be determined from the general female population. The levels of FSH during the follicular phase of the cycle range from about 2.5 U/liter to about 10.2 U/liter. At the midcycle peak, the FSH levels rise to a range from about 3.4 U/liter to about 33.4 U/liter. During the luteal phase, the FSH levels fall and range from about 1.5 U/liter to about 9.1 U/liter. Alternatively, the current levels of FSH are compared with pre-disease and/or pre-surgery levels of FSH, wherein the latter two are part of the subject's medical record.

A treatment regiment is designed that includes an FSH receptor antagonist. The FSH receptor antagonist is the aryl sulfonic acid compound 7-{4-[Bis-(2-carbamoyl-ethyl)-amino]-6-chloro-(1,3,5)-triazin-2-ylamino)-4-hydroxy-3-(4-methoxy-phenylazo)-naphthalene}-2-sulfonic acid described in Arey, et al. Endocrinol. 143:3822-3829, 2002, which is incorporated herein by reference. The efficacious dosage to be used in the treatment regimen is subjectively determined by the attending physician. The variables involved include the current levels of FSH, the size, the age, the degree of osteoporosis disease and the response pattern of the patient. In therapeutic treatment, daily dosages of the aryl sulfonic acid compound in single or multiple oral doses total 0.1-500 mg/kg. See, e.g., U.S. Pat. No. 6,355,633, which is incorporated herein by reference. Daily dosing with the aryl sulfonic acid compound is part of a 28 day cycle of drug administration. The aryl sulfonic acid compound is administered daily for 21 to 24 days at doses ranging from about 0.1 mg/kg to about 500 mg/kg, followed by 4 to 7 days of dosing with a substantially reduced dose of the aryl sulfonic acid compound or with a sugar pill or no dosing at all (“drug holiday”). During the 4 to 7 days of reduced or absent doses of the aryl sulfonic acid compound, the FSH levels rise, inducing a spike in FSH levels that simulates target pre-disease cycling of FSH levels. The treatment regimen includes multiple 28 day cycles over the course of months to years.

The levels of FSH in the blood of the female subject may or may not decrease in response to an FSH receptor antagonist as the latter is not directly altering the synthesis and/or secretion of FSH. However, the bioactivity of endogenous FSH is reduced because the activity of the FSH receptor and associated down stream signaling events are inhibited. The effects of the treatment regiment are monitored by assessing FSH receptor mediated signaling events including reductions in osteoclast bone resorption. A decrease in bone resorption mediated by the treatment regimen is monitored by periodically measuring the serum levels of TRAP, bone alkaline phosphatase, and/or other bone markers during the course of treatment. In addition, the pDXA scan is periodically repeated over the course of treatment to assess the effects of the treatment regimen on bone mineral density. Based on the serum levels of TRAP, alkaline phosphatase and/or other bone markers and the x-ray scan, the physician can choose to adjust the treatment regimen by either changing the daily dose of the FSH receptor antagonist or by changing the dosing schedule over the 28 day dosing cycle.

### Example 3

### Follicle-Stimulating Hormone Inhibitor and Follicle-Stimulating Hormone Receptor Antagonist Combination Treatment Regimen in Female Subject with Paget's Bone Disease

A treatment regiment is described that includes the combination of a follicle-stimulating hormone inhibitor and a follicle-stimulating hormone receptor antagonist to treat a perimenopausal female subject diagnosed with Paget's bone disease. The treatment regimen is based on the pre-disease levels of FSH found generally in the female population, and on the current levels of FSH in the female subject. The female subject is diagnosed with Paget's bone disease by her primary care physician, her endocrinologist, and/or her orthopedic physician using x-ray imaging, blood tests, and a bone scan. For the bone scan, the female subject is admitted to the nuclear medicine department where she is injected with 10-15 mCi of the radioactive compound Technetium-99m-methylenediphosphonate. After about two to four hours, the female subject is imaged using a gamma camera and abnormally high accumulations (hot spots) of the radioactive tracer are documented. Intense uptake of radioactive tracer involving large areas of the skeleton or the whole of a bone with curvature in the long axis is indicative of Paget's bone disease. See, e.g., Tang & Chan, Singapore Medical Journal 24:61-72, 1982, which is incorporated herein by reference. In addition, the baseline levels of one or more markers of bone resorption are assessed in the serum of the female subject for use in diagnosis of Paget's bone disease and for use in monitoring treatment efficacy. In this case study, bone-specific alkaline phosphatase (BAP) is measured in the blood of the female subject by an immunoradiometric assay using a commercially available diagnostic kit (Hybritech Ostase®, Beckman Coulter, Fullerton, Calif.). Normal values of BAP is premenopausal women ranges from about 2.9 mg/L to about 14.5 mg/L and in postmenopausal women ranges from about 3.8 mg/L to about 22.6 mg/L. Elevated levels of BAP are correlated with increased bone turnover and resorption and levels that are more than twice the normal range of BAP in an age matched individual are indicative of Paget's bone disease. Elevated levels of BAP at about 18.0 mg/L are measured in the perimenopausal female subject.

The treatment regimen is based on the pre-disease levels of FSH found generally in the female population. The current levels of FSH in the female subject are measured by time-resolved immunofluorometric assay using one or more FSH-specific antibodies labeled with the lanthanide chelate europium(III). For the assay, blood is drawn from the female subject using standard venipuncture techniques into a glass vacuum tube in the presence of one or more anti-coagulants (e.g., heparin, EDTA, sodium citrate). Plasma is isolated from the whole blood by centrifugation at 900×g for 15 minutes at room temperature. After centrifugation, the top layer containing the plasma is removed. The plasma sample is added to one or more wells of a 96 well assay plate previously coated with a first antibody directed against FSH. The assay plate is washed to remove unbound FSH and further incubated with a second FSH antibody labeled with europium (III). After additional washing, the samples are measured in a plate reading, time-resolved fluorometer such as, for example, the EnVision™ multilabel fluorometer (from, PerkinElmer Life Sciences, Boston, Mass.). FSH standards of known concentration are used to generate a standard curve for comparison with the FSH in the plasma sample. See, e.g., Bador, et al., Clin. Chem. 33:48-51, 1987, which is incorporated herein by reference.

A treatment regimen is designed that includes an FSH inhibitor and an FSH receptor antagonist. The treatment regimen includes an FSH inhibitor, elagolix (4-[[(1R)-2-[5-(2-Fluoro-3-methoxyphenyl)-3-[[2-fluoro-6-(trifluoromethyl)phenyl]methyl]-3,6-dihydro-4-methyl-2,6-dioxo-1(2H)-pyrimidinyl]-1-phenylethyl]amino]butanoic acid), which is an antagonist of gonadotrophin releasing hormone that inhibits release of FSH. The treatment regimen includes an FSH receptor antagonist, an aryl sulfonic acid compound, 7-{4-[Bis-(2-carbamoyl-ethyl)-amino]-6-chloro-(1,3,5)-triazin-2-ylamino)-4-hydroxy-3-(4-methoxy-phenylazo)-naphthalene}-2-sulfonic acid. See, e.g., Arey, et al. Endocrinol. 143:3822-3829, 2002, which is incorporated herein by reference. The therapeutically effective dosage to be used in the treatment regimen is subjectively determined by the attending physician. The variables in determining the therapeutically effective dosage in the treatment regimen include the current levels of FSH relative to the pre-disease levels of FSH (pre-disease levels in the general population or in the female subject), the size and the age of the female subject, the degree of osteoporosis disease and the response pattern of the female subject. The elagolix is administered as a daily oral dose ranging from about 75 mg to about 150 mg. The aryl sulfonic acid compound is administered as single or multiple oral doses totaling 0.1-500 mg/kg per day. See, e.g., U.S. Pat. No. 6,355,633, which is incorporated herein by reference. Daily dosing with elagolix and the aryl sulfonic acid compound is part of a 28 day cycle of drug administration that includes 21 to 24 days of daily dosing with about 75 mg to about 150 mg of elagolix and about 0.1 mg/kg to about 500 mg/kg of the aryl sulfonic acid compound, followed by 4 to 7 days of dosing with a sugar pill or no dosing at all (“drug holiday”). During the 4 to 7 days in the absence of elagolix and the aryl sulfonic acid compound, the FSH levels rise, inducing a spike in FSH levels that simulates pre-disease cycling of FSH levels. The treatment regimen includes multiple 28 day cycles over the course of months to years.

The levels of FSH in the serum of the female subject are monitored on multiple days over the course of several 28 day treatment cycles to verify that the treatment regimen is lowering the FSH levels and that the periodic “drug holiday” is inducing a cyclic spike in FSH level. FSH can be measured in the serum of the female subject as described above using a immunofluorometric assay system. If needed, treatment with the FSH inhibitor and/or the FSH receptor antagonist is adjusted either in terms of dosage or in terms of timing to achieve cyclic levels of FSH approaching target cyclic physiological pre-disease levels. In addition, treatment efficacy with the FSH inhibitor and the FSH receptor antagonist on the Paget's bone disease can be monitored by reassessing the levels of one or more markers of bone resorption, e.g., BAP, and compared with the levels measured prior to the initiation of the treatment regimen. A decrease in the serum BAP levels in response to the treatment regimen is indicative of decreased rate of bone resorption. In addition, the bone scan is repeated at least 6 months after the initiation of treatment to determine whether the treatment regimen has decreased the rate of bone turnover.

### Example 4

### Combination Treatment Including Follicle Stimulating Hormone Inhibitor and an Osteoporosis Medication in Female Subject with Osteoporosis Disease

A treatment regimen is described that includes the combination of a follicle-stimulating hormone (FSH) inhibitor and an osteoporosis medication to treat a perimenopausal or postmenopausal female subject diagnosed with osteoporosis disease. The diagnosis of osteoporosis is made based on bone mineral density of the female subject's hip and spinal cord as measured by dual energy X-ray absorptiometry (DXA scan). The bone mineral density of the female subject is compared to that of a healthy adult women 20-30 years of age and the resulting standard deviation or T score is lower than −2.5, indicative of osteoporosis as defined by the World Health Organization. In addition, the baseline levels of one or more markers of bone resorption are assessed in the serum of the female subject for use in diagnosis of osteoporosis and for use in monitoring treatment efficacy. In this example, the serum levels of cross-linked N-telopeptides of type I collagen (NTx) are used as part of the diagnosis. Serum is isolated from clotted whole blood and quantitative analysis of NTx is performed using a commercially available ELISA-based diagnostic kit (e.g., Osteomark® NTx Serum, from Inverness Medical Innovations, Waltham, Mass.). NTx is recorded in units of Bone Collagen Equivalents (BCE) and ranges from 6.2 nm to 19.0 nm BCE in normal, premenopausal women. Serum levels above this normal range are indicative of high bone turnover and osteoporosis.

The treatment regimen is based on the current and the pre-disease levels of FSH of the female subject, or on the pre-disease levels of FSH found generally in the female population. The current levels of FSH in the female subject are measured by electrogenerated chemiluminescence immunoassay in which electrical stimulation causes a bound label reagent to emit light. In this assay system, magnetic particles containing a chemiluminescent label, e.g., Ru²⁺ (tris-bipyridyl ruthenium metal cation) are reacted with the sample to form an immunocomplex. The immunocomplex is drawn to an electrode by the action of a magnet and the immunocomplex emits light when the appropriate voltage is applied. See, e.g., Imai, et al., Hitachi Rev. 57 (1): January 2008, which is incorporated herein by reference. Blood is drawn from the female subject using standard venipuncture techniques and serum is collected free of clots, cells and other particulate material as described herein. The serum sample is analyzed for FSH levels using an integrated diagnostic electrogenerated chemiluminescence system, e.g., the Cobas®6000 with the cobas e 601 immunoassay analyzer (Roche Diagnostics, F. Hoffmann-La Roche A G, Basel, Switzerland). The levels of FSH in the female subject range from about 25 U/liter to about 75 U/liter. These levels, in combination with the age of the subject, and the cessation of menses are indicative of a postmenopausal state. The current levels of FSH are compared with pre-disease levels of FSH, which is part of the subject's medical record.

A treatment regimen is designed that includes an inhibitor of FSH and an osteoporosis medication. In this example, the inhibitor of FSH is cetrorelix, a decapeptide antagonist of gonadotropin releasing hormone (GnRH). The GnRH antagonist inhibits the release of FSH from the anterior pituitary in a dose dependent manner. The osteoporosis medication is risedronate ([1-hydroxy-2-(3-pyridinyl)ethylidene]bis[phosphonic acid]; ACTONEL®), a pyridinyl bisphosphonate compound that inhibits osteoclast-mediated bone resorption and modulates bone metabolism. Cetrorelix (250 micrograms in 1 milliliter) is self administered once daily as a subcutaneous injection into the lower abdominal area at least one inch away from the navel. Risedronate is taken either once weekly (35 mg tablet) or two consecutive days monthly (75 mg tablets). Dosing with cetrorelix and risedronate is part of a 28 day cycle of drug administration that includes 21 to 24 days of daily dosing with 250 micrograms cetrorelix per day, followed by 4 to 7 days of subcutaneous dosing with saline or no dosing at all (“drug holiday”). A 35 mg tablet of risedronate is taken on day 1, day 8, day 15, and day 22 of the 28 day cycle. Alternatively, risedronate is taken on two consecutive days (75 mg each day) within the 28 day cycle, e.g., day 1 and day 2. During the 4 to 7 days in the absence of cetrorelix, the FSH levels rise, inducing a spike in FSH levels to achieve cyclic levels of FSH approaching target cyclic physiological premenopausal levels. The treatment regimen with cetrorelix and risendronate includes multiple 28 day cycles over the course of months to years.

The levels of FSH in the serum of the female subject are monitored on multiple days over the course of several 28 day cycles to verify that the treatment regimen is lowering the FSH levels and that the periodic “drug holiday” is inducing a cyclic spike in FSH level. FSH can be measured in the serum of the female subject as described above. In addition, the treatment efficacy of the FSH modulator and the osteoporosis medication on the osteoporosis disease can be monitored by reassessing one or more markers of bone resorption, e.g., NTx, and comparing these values with values measured prior to the initiation of the treatment regimen. A decrease in the serum NTx levels in response to the treatment regimen is indicative of decreased rate of bone turnover. Changes in bone mineral density as measured by a DXA scan or other imaging modality can also be used to monitor the efficacy of the treatment regimen. Based on the serum levels of one or more bone markers and the x-ray scan, the physician can choose to adjust the treatment regimen by either changing the daily dose of the FSH inhibitor and/or osteoporosis medication or by changing the dosing schedule over the 28 day dosing cycle.

### Example 5

### Combination Follicle-Stimulating Hormone Inhibitor and Steroid Hormone Composition to Prevent Osteoporosis Disease in a Female Undergoing Oophorectomy

A treatment regimen is described that includes a follicle stimulating hormone (FSH) inhibitor in combination with a steroid hormone composition or steroid hormone modulator composition for preventing osteoporosis in a premenopausal female subject who has undergone bilateral oophorectomy. The treatment regimen is based on the current and the pre-oophorectomy levels of FSH of the female subject. The treatment regimen is further based on the current and pre-oophorectomy levels of at least one steroid hormone, e.g., estradiol. The female subject undergoes a hysterectomy and bilateral salpingo-oophorectomy for non-malignant disease in her mid-thirties while still premenopausal. Oophorectomy in combination with hysterectomy in premenopausal women induces an immediate decline in estrogen and is linked to a higher risk of osteoporosis 3 to 6 years post surgery as compared to similar aged women who undergo a hysterectomy alone. See, e.g., Aitken, et al., Br. Med. J. 2: 325-328, 1973, which is incorporated herein by reference. The bone mineral density and various bone markers of the female subject are assessed prior to surgery as a reference point for her bone health. The bone mineral density is measured using a whole body dual energy x-ray absorptiometry (DXA) scan. In addition, various blood tests for bone alkaline phosphatase (BAP), tartrate resistant acid phosphatase (TRAP), cross-linked N-telopeptides of type I collagen (NTx), and other markers of bone health are assessed using the various methods described herein.

The treatment regimen is based on the current and the pre-oophorectomy levels of FSH of the female subject, or on the pre-disease levels of FSH found generally in the female population. The FSH and estradiol levels of the female subject are assessed pre-oophorectomy and post-oophorectomy using isolated serum and a chemiluminescence immunoassay (CLIA). In this assay system, one or more immunoreagents in the assay are labeled with horseradish peroxidase that catalyzes oxidation of a luminol-based substrate resulting in a light-emitting enzymatic reaction. Light emission is detected using a luminometer and is directly proportional to the level of hormone in the serum sample. Blood is drawn from the female subject using standard venipuncture techniques and serum is collected free of clots, cells and other particulate material as described herein. Aliquots of the serum are assayed separately for FSH and estradiol using commercial CLIA diagnostic systems as described by the manufacturer (e.g. AccuLite® FSH CLIA and AccuLite® Estradiol (E2) CLIA from Monobind, Inc., Lake Forest, Calif.). The levels of FSH in the female subject prior to oophorectomy range from about 2 U/liter to about 22 U/liter depending upon the time of assay during the menstrual cycle. The levels of FSH in the female subject following oophorectomy and prior to treatment range from about 35 U/liter and about 150 U/liter. The levels of estradiol in the female subject prior to oophorectomy range from about 9 pg/ml to about 281 pg/ml, depending upon the time of assay during the menstrual cycle. The levels of estradiol in the female subject following oophorectomy and prior to replacement therapy range from undetectable to about 20 pg/ml.

A treatment regimen is designed that includes an FSH inhibitor and at least one steroid hormone or steroid hormone modulator. The FSH inhibitor is an antagonist of gonadotropin releasing hormone (GnRH). The GnRH antagonist inhibits the release of FSH from the anterior pituitary in a dose-dependent manner. A GnRH antagonist for use in reducing serum levels of FSH is the synthetic decapeptide ganirelix (Orgalutran®). Ganirelix (250 micrograms in 500 microliters) is self-administered once daily as a subcutaneous injection into the upper thigh or into the abdomen around the navel. Daily dosing with ganirelix is part of a 28 day cycle of drug administration that includes 21 to 24 days of daily dosing with 250 micrograms ganirelix per day, followed by 4 to 7 days of subcutaneous dosing with saline or no dosing at all (“drug holiday”). During the 4 to 7 days in the absence of ganirelix, the FSH levels rise, inducing a spike in FSH levels that simulates premenopausal cycling of FSH levels. The treatment regimen further includes at least one steroid hormone. Estradiol is used for replacement therapy. Estradiol formulations come in many forms including oral tablets, topical cream or gel, transdermal patch, implant, and vaginal ring. The treatment regiment includes a topical gel formulation, e.g., EstroGel® estradiol gel (Ascend Therapeutics, Herndon, Va.). EstroGel® estradiol gel is administered to the skin of the female subject once daily from a metered pump, with each 1.25 g dose of gel containing up to 0.75 mg of estradiol. The estradiol is administered once daily over the course of the 28 day treatment cycle. If appropriate, higher doses of estradiol can be achieved by using multiple daily dosing. The treatment regimen of FSH inhibitor and estradiol includes multiple 28 day cycles over the course of months to years to prevent osteoporosis.

The levels of FSH in the serum of the female subject are monitored on multiple days over the course of several 28 day cycles to verify that the treatment regimen is lowering the FSH levels and that the periodic “drug holiday” is inducing a cyclic spike in FSH level. FSH is measured in the serum of the female subject as described above. If needed, treatment with the GnRH antagonist is adjusted either in terms of dosage or in terms of timing to achieve cyclic levels of FSH comparable to levels observed pre-oophorectomy. Estradiol is also measured over the course of the treatment regimen to assess whether the current serum levels are at or near the pre-oophorectomy levels. In addition, the efficacy of treatment with the FSH modulator and estradiol can be monitored by reassessing one or more markers of bone resorption, e.g., BAP, TRAP and/or NTx and comparing these values with values measured prior to the initiation of the treatment regimen. A decrease in the serum bone markers in response to the treatment regimen is indicative of decreased rate of bone turnover. Changes in bone mineral density as measured by a DXA scan or other imaging modality can also be used to monitor the efficacy of the treatment regimen. Based on the serum levels of one or more bone markers and the x-ray scan, the physician can choose to adjust the treatment regimen by either changing the daily dose of the FSH inhibitor and/or steroid hormone or by changing the dosing schedule over the 28 day dosing cycle.

### Example 6

### Combination Treatment Including Follicle-Stimulating Hormone Inhibitor, Follicle-Stimulating Hormone Receptor Antagonist, and Steroid Hormone Composition Administered to a Female Subject with Osteoporosis Disease

A treatment regimen is described that includes a follicle-stimulating hormone inhibitor, a follicle-stimulating hormone receptor antagonist, in combination with a steroid hormone composition or steroid hormone modulator composition for treating a perimenopausal or postmenopausal female subject diagnosed with osteoporosis disease. The treatment regimen is based on the current and the pre-disease levels of FSH of the female subject. The treatment regimen is further based on the current and the pre-disease levels of at least one steroid hormone, e.g., estradiol, of the female subject. In some aspects, the pre-disease levels of FSH and/or estradiol of the female subject are synonymous with premenopausal levels. The female subject is diagnosed with osteoporosis by her primary care physician and her endocrinologist. The diagnosis of osteoporosis is made based on the bone mineral density of the female subject's wrist, heel, and/or finger as measured by peripheral dual energy x-ray absorptiometry (pDXA). The bone mineral density of the female subject is compared to that of a healthy adult women 20-30 years of age and the resulting standard deviation or T score is lower than −2.5, indicative of osteoporosis as defined by the World Health Organization. In addition, one or more markers of bone turnover are assayed to confirm the osteoporosis diagnosis and for use in monitoring treatment efficacy. Assays for serum or urine levels of bone alkaline phosphatase (BAP), tartrate resistant acid phosphatase (TRAP), cross-linked N-telopeptides of type I collagen (NTx), and other markers of bone health are assessed using the various methods described herein. Elevated levels of BAP, TRAP, and/or NTx are indicative of increased bone turnover and resorption, which are leading symptoms of osteoporosis.

The current levels of FSH and estradiol levels in the female subject are measured by electrogenerated chemiluminescence immunoassay in which electrical stimulation causes a bound label reagent to emit light. In this assay system, magnetic particles containing a chemiluminescent label, e.g., Ru²⁺ (tris-bipyridyl ruthenium metal cation) are reacted with the sample to form an immunocomplex. The immunocomplex is drawn to an electrode by the action of a magnet, and the immunocomplex emits light when the appropriate voltage is applied. See, e.g., Imai, et al., Hitachi Rev. 57: January 2008, which is incorporated herein by reference. Blood is drawn from the female subject using standard venipuncture techniques and serum is collected free of clots, cells and other particulate material as described herein. The serum sample is analyzed for FSH and estradiol levels using an integrated diagnostic electrogenerated chemiluminescence system, e.g., the Cobas®6000 immunoassay analyzer with the cobas e 601 module (Roche Diagnostics; F. Hoffmann-La Roche AG, Basel, Switzerland). The levels of FSH in the female subject range from about 25 U/liter to about 75 U/liter. The levels of estradiol in the female subject range from about undetectable to about 20 pg/ml. These levels of FSH and estradiol, in combination with the age of the subject and the cessation of menses, are indicative of a postmenopausal state. The current levels of FSH and estradiol are compared with pre-disease levels of FSH and estradiol, which are part of the subject's medical record. Alternatively, cyclic physiological pre-disease levels of FSH can be determined from the general female population.

A treatment regimen is designed that includes a combination of an FSH inhibitor and an FSH receptor antagonist. The treatment regimen further includes at least one steroid hormone or steroid hormone modulator. The FSH inhibitor is elagolix (4-[[(1R)-2-[5-(2-Fluoro-3-methoxyphenyl)-3-[[2-fluoro-6-(trifluoromethyl)phenyl]methyl]-3,6-dihydro-4-methyl-2,6-dioxo-1(2H)-pyrimidinyl]-1-phenylethyl]amino]butanoic acid), a gonadotrophin releasing hormone antagonist that inhibits release of FSH. The FSH receptor antagonist is the aryl sulfonic acid compound 7-{4-[Bis-(2-carbamoyl-ethyl)-amino]-6-chloro-(1,3,5)-triazin-2-ylamino)-4-hydroxy-3-(4-methoxy-phenylazo)-naphthalene}-2-sulfonic acid. See, e.g., Arey, et al. Endocrinol. 143:3822-3829, 2002, which is incorporated herein by reference. The treatment regimen further includes at least one steroid hormone that is the composition PREMPRO® conjugated estrogens and progesterone derivative medroxyprogesterone acetate.

The therapeutically effective dosage to be used in the treatment regimen is subjectively determined by the attending physician based on the physiological condition of the female subject. The variables include the current and pre-disease levels of FSH in the female subject, the current and pre-disease levels of estradiol, the size, the age, the degree of osteoporosis disease and the response pattern of the female subject. The treatment regimen includes daily dosing with elagolix, the aryl sulfonic acid compound, and PREMPRO® conjugated estrogens and progesterone derivative medroxyprogesterone acetate as part of a 28 day cycle of drug administration. The 28 day cycle includes 21 to 24 days of daily dosing with about 75 mg to about 150 mg of elagolix and about 0.1 mg/kg to about 500 mg/kg of the aryl sulfonic acid compound, followed by 4 to 7 days of dosing with a sugar pill or no dosing at all (“drug holiday”). During the 4 to 7 days in the absence of elagolix and the aryl sulfonic acid compound, the FSH levels rise, inducing a spike in FSH levels that simulates pre-disease cycling of FSH levels. The PREMPRO® is administered once daily over the course of the 28 day treatment cycle as an oral tablet with estrogen/medroxyprogesterone acetate doses of 0.3 mg/1.5 mg, 0.45 mg/1.5 mg, 0.625 mg/2.5 mg, or 0.625 mg/5 mg. If appropriate, higher doses of PREMPRO® can be achieved by using multiple daily dosing. The treatment regimen of FSH inhibitor, FSH receptor antagonist and steroid hormone includes multiple 28 day cycles over the course of months to years to prevent osteoporosis.

The levels of FSH in the serum of the female subject are monitored on multiple days over the course of several 28 day cycles to verify that the treatment regimen is lowering the FSH levels and that the periodic “drug holiday” is inducing a cyclic spike in FSH level. FSH can be measured in the serum of the female subject as described above. If needed, treatment with the FSH inhibitor and FSH receptor antagonist is adjusted either in terms of dosage or in terms of timing to achieve cyclic levels of FSH comparable to cyclic physiological pre-disease levels or premenopausal levels. Estradiol is also measured over the course of the treatment regimen to assess whether the current serum levels are at or near the pre-disease levels. In addition, the efficacy of treatment with the FSH modulator and estradiol can be monitored by reassessing one or more markers of bone resorption, e.g., BAP, TRAP and/or NTx and comparing these values with values measured prior to the initiation of the treatment regimen. A decrease in the serum bone markers in response to the treatment regimen is indicative of decreased rate of bone turnover. Changes in bone mineral density as measured by one or more pDXA scan or other imaging modality can also be used to monitor the efficacy of the treatment regimen. Based on the serum levels of one or more bone markers and the x-ray scan, the physician can choose to adjust the treatment regimen by either changing the daily dose of the FSH inhibitor, FSH receptor antagonist and/or steroid hormone or by changing the dosing schedule over the 28 day dosing cycle.

### Example 7

### Device Useful for Sensing and Administering a Combination Treatment Regimen Including Follicle-Stimulating Hormone Inhibitor, Follicle-Stimulating Hormone Receptor Antagonist, and Steroid Hormone Composition Administered to a Female Subject with Osteoporosis Disease

A treatment regimen for treating osteoporosis disease in a female subject includes a device for sensing one or more hormone and administering a combination of at least one FSH inhibitor, at least one FSH receptor antagonist, and at least one steroid hormone composition configured to reduce levels of FSH or reduce FSH bioactivity or bioavailability to approach a target cyclic physiological pre-disease level of FSH. The treatment regimen reduces osteoporosis disease in the female subject. The female subject is diagnosed with osteoporosis by her primary care physician and her endocrinologist. The diagnosis of osteoporosis is made based on dual energy x-ray absorptiometry (DXA). The female subject is found to have a bone mineral density with a standard deviation relative to young, normal women lower than −2.5, indicative of osteoporosis. In addition, one or more markers of bone turnover are assayed to confirm the osteoporosis diagnosis and for use in monitoring treatment efficacy. Assays for serum or urine levels of bone alkaline phosphatase (BAP), tartrate resistant acid phosphatase (TRAP), cross-linked N-telopeptides of type I collagen (NTx), and other markers of bone health are assessed using the various methods described herein. Elevated levels of BAP, TRAP, and/or NTx are indicative of increased bone turnover and resorption, and are indicative of osteoporosis disease.

The treatment regimen is based on the current and the pre-disease levels of FSH of the female subject, or on the pre-disease levels of FSH found generally in the female population. The treatment regimen is further based on the current and the pre-disease levels of at least one steroid hormone, e.g., estradiol, of the female subject, or on the pre-disease levels of FSH found generally in the female population. In some aspects, the pre-disease levels of FSH and/or estradiol of the female subject are synonymous with premenopausal levels. The female subject is fitted with a device for monitoring FSH and estradiol and for delivering a treatment regimen that includes a FSH inhibitor, a FSH receptor antagonist, and estradiol. The device is worn in contact with the surface of the subject's skin to enable direct blood sampling as well as direct administration of the treatment regimen. The device is affixed to an area of skin located on the lower abdomen of the female subject, but 2-3 inches removed from the navel. The device includes an array of microneedles for blood sampling, multiple microchip sensors, a controller, and a drug delivery system that includes an infusion pump.

The device includes one or more sensors for sensing FSH and estradiol in the peripheral blood of the female subject. For blood sampling, a small microneedle is used to perforate the skin and draw up a small sample of blood by capillary action. The blood is drawn up into a microchip that includes one or more sensors to sense the levels of FSH and/or estradiol in the subject's blood sample. The microchip sensor includes recognition elements (e.g., antibodies) that are specific for FSH and estradiol, respectively. Binding of FSH and estradiol to their respective recognition elements generates an electrical signal that is sent to a controller associated with the device. Blood samples are monitored on a daily basis, preferably at the same time each day (e.g., upon rising in the morning) to account for possible circadian fluctuations in hormone levels. A separate microneedle is used for each of the daily blood draws. The device includes an array of microneedles, with each microneedle linked to its own microchip sensor such that any given microchip sensor is only used once. The device includes a clock mechanism that automatically triggers a daily blood draw from consecutive needles every 24 hours. Alternative timing of blood sampling is also possible, depending upon the needs of the subject.

The sensors associated with the microchip sensor send electrical signals to the controller in response to binding FSH and estradiol in the female subject's blood sample. The controller compares the current levels with historical levels of FSH and estradiol of the female subject. The controller includes stored data regarding time-history profiles of FSH and estradiol gathered premenopause, pre-disease, at diagnosis, at initiation of treatment, and since the initiation of treatment. The controller also includes stored data regarding the levels of FSH and estradiol in population norms including those of premenopausal women and those of age-matched, disease-free women. The controller also includes stored data that indicates the target cyclic physiological pre-disease levels of FSH of the female subject, measured over one or more 28 day cycles. The clock associated with the controller keeps track of the 28 day cycle and based on the current levels of hormones and the stored data, the controller triggers delivery of the appropriate amount of FSH inhibitor, FSH receptor antagonists, and estradiol.

The device includes reservoirs for storing and delivering one or more FSH inhibitors, one or more FSH receptor antagonists, and estradiol. The reservoirs are linked through an infusion pump to a common outflow tube into an infusion set inserted via a metal or Teflon needle into the subject. Upon signaling from the controller, an appropriate dose of each component of the treatment regimen is delivered to the subject via the infusion pump/infusion set. The FSH inhibitor is elagolix (4-[[(1R)-2-[5-(2-Fluoro-3-methoxyphenyl)-3-[[2-fluoro-6-(trifluoromethyl)phenyl]methyl]-3,6-dihydro-4-methyl-2,6-dioxo-1(2H)-pyrimidinyl]-1-phenylethyl]amino]butanoic acid), which is a gonadotrophin-releasing hormone antagonist that inhibits release of FSH. The FSH receptor antagonist is the aryl sulfonic acid compound 7-{4-[Bis-(2-carbamoyl-ethyl)-amino]-6-chloro-(1,3,5)-triazin-2-ylamino)-4-hydroxy-3-(4-methoxy-phenylazo)-naphthalene}-2-sulfonic. See, e.g., Arey, et al. Endocrinol. 143:3822-3829, 2002, which is incorporated herein by reference. The treatment regimen further includes estradiol, e.g., EstroGel® estradiol gel (Ascend Therapeutics, Herndon, Va.).

The therapeutically effective dosage to be used in the treatment regimen is determined by the controller based on a number of variables including the current and pre-disease levels of FSH, the current and pre-disease levels of estradiol, the size, the age, the degree of osteoporosis disease and the response pattern of the female subject. The treatment regimen includes daily infusion with elagolix, the aryl sulfonic acid compound, and estradiol as part of a 28 day cycle of drug administration. The 28 day cycle includes 21 to 24 days of daily infusion with about 10 mg to about 150 mg of elagolix and about 0.1 mg/kg to about 500 mg/kg of the aryl sulfonic acid compound, followed by 4 to 7 days of infusion with saline (“drug holiday”). During the 4 to 7 days in the absence of elagolix and the aryl sulfonic acid compound, the FSH levels rise, inducing a spike in FSH levels that simulates pre-disease cycling of FSH levels. The estradiol is administered by daily infusion over the course of the 28 day treatment cycle at doses ranging from about 0.01 mg/day to about 0.1 mg/day. The treatment regimen of FSH inhibitor, FSH receptor antagonist and estradiol includes multiple 28 day cycles over the course of months to years to treat osteoporosis.

The levels of FSH and estradiol in the blood of the female subject are monitored by the device on a daily basis over the course of the 28 day cycle to verify that the treatment regimen is lowering the FSH levels and that the periodic “drug holiday” is inducing a cyclic spike in FSH levels. If needed, the controller alters the dose of elagolix, the aryl sulfonic acid compound, estradiol, or combinations thereof to achieve cyclic physiological levels of FSH comparable to pre-disease levels or premenopausal levels. In addition, the device can be configured to monitor one or more markers of bone resorption as a measure of treatment efficacy. The levels of BAP, TRAP and/or NTx are measured periodically over the course of treatment and the data stored in the device. The controller compares the current levels of the bone markers with those levels measured prior to the initiation of the treatment. Based on this comparison, the controller can initiate changes in delivery of elagolix, the aryl sulfonic acid compound, estradiol, or combinations thereof. Changes in bone mineral density as measured by one or more DXA scan or other imaging modality can also be used to monitor the efficacy of the treatment regimen. This data can also be used to make adjustments in the treatment regimen.

Each recited range includes all combinations and sub-combinations of ranges, as well as specific numerals contained therein.

All publications and patent applications cited in this specification are herein incorporated by reference to the extent not inconsistent with the description herein and for all purposes as if each individual publication or patent application were specifically and individually indicated to be incorporated by reference for all purposes.

The state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Optical aspects of implementations will typically employ optically-oriented hardware, software, and or firmware.

In a general sense the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). The subject matter described herein may be implemented in an analog or digital fashion or some combination thereof.

The herein described components (e.g., steps), devices, and objects and the description accompanying them are used as examples for the sake of conceptual clarity and that various configuration modifications using the disclosure provided herein are within the skill of those in the art. Consequently, as used herein, the specific examples set forth and the accompanying description are intended to be representative of their more general classes. In general, use of any specific example herein is also intended to be representative of its class, and the non-inclusion of such specific components (e.g., steps), devices, and objects herein should not be taken as indicating that limitation is desired.

With respect to the use of substantially any plural or singular terms herein, the reader can translate from the plural to the singular or from the singular to the plural as is appropriate to the context or application. The various singular/plural permutations are not expressly set forth herein for sake of clarity.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable or physically interacting components or wirelessly interactable or wirelessly interacting components or logically interacting or logically interactable components.

While particular aspects of the present subject matter described herein have been shown and described, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an”; the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, or A, B, and C together, etc.). Virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art after reading the disclosure herein. The various aspects and embodiments disclosed herein are for purposes of illustration and are not intended to be limiting, with the true scope and spirit being indicated by the following claims.

What is claimed is:

1. A method for treating a bone loss disease or a bone loss disorder in a mammalian subject comprising:
providing to the mammalian subject at least one treatment regimen including at least one follicle-stimulating hormone modulator configured to and in an amount sufficient to reduce bioactivity or bioavailability of follicle-stimulating hormone in the mammalian subject, and to approximate the level of bioactive or bioavailable follicle-stimulating hormone to a target cyclic physiological pre-disease effective level in the mammalian subject.

2. The method of claim 1, wherein the at least one follicle-stimulating hormone modulator includes an inhibitor of follicle-stimulating hormone bioactivity.

3. The method of claim 1, wherein the at least one follicle-stimulating hormone modulator includes a follicle-stimulating hormone receptor antagonist.

4. The method of claim 1, wherein the at least one follicle-stimulating hormone modulator includes an inhibitor of osteoclast activity.

5. The method of claim 1, wherein the at least one follicle-stimulating hormone modulator includes a small chemical molecule, polypeptide, nucleic acid, or antibody.

6. The method of claim 1, wherein the at least one treatment regimen further includes providing replacement therapy including one or more steroid hormones or metabolites or modulators thereof.

7. The method of claim 6, wherein the at least one treatment regimen is determined based on population data of physiological cyclic pre-disease levels of the one or more steroid hormones in one or more mammalian subjects.

8. The method of claim 6, wherein the at least one treatment regimen including the least one replacement therapy is configured to increase levels of one or more of an estrogen or a progestogen, or metabolites or modulators thereof.

9. The method of claim 6, wherein the at least one treatment regimen includes replacement therapy with one or more of an estrogen or a progestogen.

10. The method of claim 6, wherein the at least one treatment regimen is determined based on pre-disease cyclic levels of steroid hormone in the mammalian subject and on current cyclic levels of steroid hormone in the mammalian subject.

11. The method of claim 1, wherein the at least one treatment regimen is determined based on pre-disease cyclic levels of follicle-stimulating hormone in the mammalian subject and on current cyclic levels of follicle-stimulating hormone in the mammalian subject.

12. The method of claim 1, wherein providing the at least one treatment regimen further includes providing a cyclic treatment regimen including at least one gonadotropin-releasing hormone modulator.

13. The method of claim 6, wherein the target cyclic physiological pre-disease level includes cyclic pulsatile levels of one or more of gonadotropin, follicle-stimulating hormone, luteinizing hormone, gonadotropin-releasing hormone, or steroid hormones.

14. The method of claim 13, wherein the target cyclic physiological pre-disease level of the follicle-stimulating hormone is based on population data of cyclic physiological pre-disease levels of the one or more of gonadotropin, follicle-stimulating hormone, luteinizing hormone, gonadotropin-releasing hormone, or steroid hormones in one or more mammalian subjects.

15. The method of claim 1, wherein the cyclic physiological pre-disease level includes a cyclic physiological premenopausal level in the mammalian subject.

16. The method of claim 6, wherein the at least one treatment regimen is configured to maintain the subject's one or more steroid hormones or metabolites or modulators thereof at substantially physiological pre-disease levels.

17. The method of claim 1, further including determining the one or more gonadotropin levels or the one or more steroid hormones levels in the subject during a treatment period.

18. The method of claim 17, wherein the treatment period includes a time period preceding treatment with the at least one follicle-stimulating hormone modulator.

19. The method of claim 17, wherein the treatment period includes a time period during treatment with the at least one follicle-stimulating hormone modulator.

20. The method of claim 19, wherein the determining of the one or more gonadotropin levels or the one or more steroid hormones levels occurs at multiple time points during the treatment period.

21. The method of claim 1, wherein the at least one treatment regimen is determined based at least in part on one or more of a time-history of gonadotropin levels or serum steroid hormone levels in the subject, on inferred peak values or minimal values of serum gonadotropin levels or serum steroid hormone levels in the subject, on age of the subject, or on categorization relative to profiles of patient populations.

22. The method of claim 1, wherein the at least one treatment regimen is determined based at least in part on Fourier analysis of the cyclic gonadotropin levels or cyclic steroid hormone levels in the subject, or on harmonic analysis of the cyclic gonadotropin levels or the cyclic steroid hormone levels in the subject.

23. The method of claim 1, wherein the at least one treatment regimen is determined based at least in part on scaled values of the gonadotropin levels or the steroid hormone levels prior to the disease diagnosis in the subject.

24. The method of claim 23, wherein the at least one treatment regimen is determined based at least in part on the scaled value approximately equal to one.

25. The method of claim 23, wherein the at least one treatment regimen is determined based at least in part on the scaled value dependent on age of the subject.

26. (canceled)

27. The method of claim 1, wherein the at least one follicle-stimulating hormone modulator includes a gonadotropin releasing hormone antagonist, FSH inhibitor, FSH synthesis inhibitor, FSH secretion inhibitor, or FSH receptor antagonist.

28.-32. (canceled)

33. A method for preventing a bone loss disease or a bone loss disorder in a mammalian subject comprising:
providing to the mammalian subject at least one treatment regimen including at least one follicle-stimulating hormone modulator configured to and in an amount sufficient to reduce or maintain bioactivity or bioavailability of follicle-stimulating hormone in the mammalian subject, and to approximate the level of bioactive or bioavailable follicle-stimulating hormone to a target cyclic physiological disease-free effective level in the mammalian subject.

34. The method of claim 33, wherein the at least one follicle-stimulating hormone modulator includes an inhibitor of follicle-stimulating hormone bioactivity.

35. The method of claim 33, wherein the at least one follicle-stimulating hormone modulator includes a follicle-stimulating hormone receptor antagonist.

36. The method of claim 33, wherein the at least one follicle-stimulating hormone modulator includes an inhibitor of osteoclast activity.

37. The method of claim 33, wherein the at least one follicle-stimulating hormone modulator includes a small chemical molecule, polypeptide, nucleic acid, or antibody.

38. The method of claim 33, wherein the at least one treatment regimen further includes providing replacement therapy including one or more steroid hormones or metabolites or modulators thereof.

39. The method of claim 38, wherein the at least one treatment regimen is determined based on population data of physiological cyclic disease-free levels of the one or more steroid hormones in one or more mammalian subjects.

40. The method of claim 38, wherein the at least one treatment regimen including the least one replacement therapy is configured to increase levels of one or more of an estrogen or a progestogen, or metabolites or modulators thereof.

41. The method of claim 38, wherein the at least one treatment regimen includes replacement therapy with one or more of an estrogen or a progestogen.

42. The method of claim 38, wherein the at least one treatment regimen is determined based on disease-free cyclic levels of steroid hormone in the mammalian subject and on current cyclic levels of steroid hormone in the mammalian subject.

43. The method of claim 33, wherein the at least one treatment regimen is determined based on disease-free cyclic levels of follicle-stimulating hormone in the mammalian subject and on current cyclic levels of follicle-stimulating hormone in the mammalian subject.

44. The method of claim 33, wherein providing the at least one treatment regimen further includes providing a cyclic treatment regimen including at least one gonadotropin-releasing hormone modulator.

45. The method of claim 38, wherein the target cyclic physiological disease-free level includes cyclic pulsatile levels of one or more of gonadotropin, follicle-stimulating hormone, luteinizing hormone, gonadotropin-releasing hormone, or steroid hormones.

46. The method of claim 45, wherein the target cyclic physiological disease-free level of the follicle-stimulating hormone is based on population data of cyclic physiological disease-free levels of one or more of gonadotropin, follicle-stimulating hormone, luteinizing hormone, gonadotropin-releasing hormone, or steroid hormones in one or more mammalian subjects.

47. The method of claim 33, wherein the cyclic physiological disease-free level includes a cyclic physiological premenopausal level in the mammalian subject.

48. The method of claim 38, wherein the at least one treatment regimen is configured to maintain the mammalian subject's one or more gonadotropins or one or more steroid hormones or metabolites or modulators thereof at substantially physiological disease-free levels.

49. The method of claim 33, further including determining the one or more gonadotropin levels or the one or more steroid hormones levels in the subject during a treatment period.

50. The method of claim 49, wherein the treatment period includes a time period preceding treatment with the at least one follicle-stimulating hormone modulator.

51. The method of claim 49, wherein the treatment period includes a time period during treatment with the at least one follicle-stimulating hormone modulator.

52. The method of claim 51, wherein the determining of the one or more gonadotropin levels or the one or more steroid hormones levels occurs at multiple time points during the treatment period.

53. The method of claim 33, wherein the at least one treatment regimen is determined based at least in part on one or more of a time-history of serum gonadotropin levels or serum steroid hormone levels in the subject, on inferred peak values or minimal values of serum gonadotropin levels serum or steroid hormone levels in the subject, on age of the subject, or on categorization relative to profiles of patient populations.

54. The method of claim 33, wherein the at least one treatment regimen is determined based at least in part on Fourier analysis of the cyclic gonadotropin levels or the cyclic steroid hormone levels in the subject, or on harmonic analysis of the cyclic gonadotropin levels or the cyclic steroid hormone levels in the subject.

55. The method of claim 33, wherein the at least one treatment regimen is determined based at least in part on scaled values of the gonadotropin levels or the steroid hormone levels prior to the disease diagnosis in the subject.

56. The method of claim 55, wherein the at least one treatment regimen is determined based at least in part on the scaled value approximately equal to one.

57. The method of claim 55, wherein the at least one treatment regimen is determined based at least in part on the scaled value dependent on age of the subject.

58.-101. (canceled)

102. A method for treating a bone loss disease or a bone loss disorder in a mammalian subject comprising:
providing a system including a sensor configured to detect one or more hormones in one or more tissues of the mammalian subject; and a controller in communication with the sensor, wherein the controller is configured to provide to the mammalian subject at least one treatment regimen including at least one follicle-stimulating hormone modulator configured to and in an amount sufficient to reduce bioactivity or bioavailability of follicle-stimulating hormone in the mammalian subject, and to approximate the level of bioactive or bioavailable follicle-stimulating hormone to a target cyclic physiological pre-disease effective level in the mammalian subject.

103. The method of claim 102, wherein the one or more hormones includes follicle-stimulating hormone, luteinizing hormone, or steroid hormone.

104. The method of claim 103, wherein the steroid hormone includes estrogen, progestogen, or testosterone.
